SEQUENCE LISTING (57 sequences)

SEQ ID NO: 1 - DNA, 15 bp, Artificial Sequence
  Artificially synthesized oligonucleotide
  misc_feature (1)..(1): The oligonucleotide is optionally biotinylated at the noted position
gggccgggcgcggtg

SEQ ID NO: 2 - DNA, 26 bp, Artificial Sequence
  Artificially synthesized oligonucleotide
gtacctttaaagagacagagtctcgc

SEQ ID NO: 3 - DNA, 20 bp, Artificial Sequence
  Artificially synthesized oligonucleotide
  misc_feature (1)..(1): The oligonucleotide is optionally biotinylated at the noted position
gcctgtaatcccagcacttt

SEQ ID NO: 4 - DNA, 19 bp, Artificial Sequence
  Artificially synthesized oligonucleotide
gagacggagtctcgctctg

SEQ ID NO: 5 - DNA, 18 bp, Artificial Sequence
  Artificially synthesized oligonucleotide
cggagtctcgctctgtcg

SEQ ID NO: 6 - DNA, 20 bp, Artificial Sequence
  Artificially synthesized oligonucleotide
  misc_feature (1)..(1): The oligonucleotide is optionally biotinylated at the noted position
cgtgcctgtagtcccagcta

SEQ ID NO: 7 - DNA, 20 bp, Artificial Sequence
  Artificially synthesized oligonucleotide
agacggggtctcgctatgtt

SEQ ID NO: 8 - DNA, 20 bp, Artificial Sequence
  Artificially synthesized oligonucleotide
gcugacctttttgctucuca

SEQ ID NO: 9 - DNA, 26 bp, Artificial Sequence
  Artificially synthesized oligonucleotide
ttggtacgcatacgtgttgactgtga

SEQ ID NO: 10 - DNA, 22 bp, Artificial Sequence
  Artificially synthesized oligonucleotide
  misc_feature (22)..(22): The oligonucleotide is optionally biotinylated at the noted position
cacgaatttgcgtgtcatcctt

SEQ ID NO: 11 - DNA, 25 bp, Artificial Sequence
  Artificially synthesized oligonucleotide
gtgctcgcttcggcagcacatatac

SEQ ID NO: 12 - DNA, 27 bp, Artificial Sequence
  Artificially synthesized oligonucleotide
aaaaatatggaacgcttcacgaatttg

SEQ ID NO: 13 - DNA, 18 bp, Artificial Sequence
  Artificially synthesized oligonucleotide
agcagagtggcgcagcgg

SEQ ID NO: 14 - DNA, 21 bp, Artificial Sequence
  Artificially synthesized oligonucleotide
gatccatcgacctctgggtta

SEQ ID NO: 15 - DNA, 20 bp, Artificial Sequence
  Artificially synthesized oligonucleotide
ggtccctggtggccttaatt

SEQ ID NO: 16 - DNA, 20 bp, Artificial Sequence
  Artificially synthesized oligonucleotide
ttgctggtaatgggcacaca

SEQ ID NO: 17 - DNA, 20 bp, Artificial Sequence
  Artificially synthesized oligonucleotide
ttagccgggagtggtgtcgg

SEQ ID NO: 18 - DNA, 21 bp, Artificial Sequence
  Artificially synthesized oligonucleotide
acctcccgggttcacgccatt

SEQ ID NO: 19 - DNA, 41 bp, Artificial Sequence
  Artificially synthesized oligonucleotide
  misc_feature (41)..(41): v is a, c, or g
gaccacgcgtatcgatgtcgacttttttttttttttttttv

SEQ ID NO: 20 - DNA, 22 bp, Artificial Sequence
  Artificially synthesized oligonucleotide
gaccacgcgtatcgatgtcgac

SEQ ID NO: 21 - DNA, 20 bp, Artificial Sequence
  Artificially synthesized oligonucleotide
aggaacagctccggtctaca

SEQ ID NO: 22 - DNA, 20 bp, Artificial Sequence
  Artificially synthesized oligonucleotide
gatgaacccggtacctcaga

SEQ ID NO: 23 - DNA, 20 bp, Artificial Sequence
  Artificially synthesized oligonucleotide
actggccatcagagaaatgc

SEQ ID NO: 24 - DNA, 20 bp, Artificial Sequence
  Artificially synthesized oligonucleotide
cagcacctgttgtttcctga

SEQ ID NO: 25 - DNA, 24 bp, Artificial Sequence
  Artificially synthesized oligonucleotide
cgcagctaggaataatggaatagg

SEQ ID NO: 26 - DNA, 20 bp, Artificial Sequence
  Artificially synthesized oligonucleotide
gcctcagttccgaaaaccaa

SEQ ID NO: 27 - DNA, 21 bp, Artificial Sequence
  Artificially synthesized oligonucleotide
gccagaagatcctggactgat

SEQ ID NO: 28 - DNA, 20 bp, Artificial Sequence
  Artificially synthesized oligonucleotide
gtaacctgggctggcatttg

SEQ ID NO: 29 - DNA, 23 bp, Artificial Sequence
  Artificially synthesized oligonucleotide
gcagatcgatccatgcttatcac

SEQ ID NO: 30 - DNA, 21 bp, Artificial Sequence
  Artificially synthesized oligonucleotide
gatgtggaggtccttgatcca

SEQ ID NO: 31 - DNA, 23 bp, Artificial Sequence
  Artificially synthesized oligonucleotide
gctcctctaaccttactaaacac

SEQ ID NO: 32 - DNA, 20 bp, Artificial Sequence
  Artificially synthesized oligonucleotide
tttgccacatttattgcact

SEQ ID NO: 33 - DNA, 20 bp, Artificial Sequence
  Artificially synthesized oligonucleotide
atagaagcctttcatcacct

SEQ ID NO: 34 - DNA, 20 bp, Artificial Sequence
  Artificially synthesized oligonucleotide
ttgccctgaaatacagttcc

SEQ ID NO: 35 - DNA, 20 bp, Artificial Sequence
  Artificially synthesized oligonucleotide
cccaaaagagacggactgct

SEQ ID NO: 36 - DNA, 19 bp, Artificial Sequence
  Artificially synthesized oligonucleotide
agcccatgcctatatgctt

SEQ ID NO: 37 - DNA, 21 bp, Artificial Sequence
  Artificially synthesized oligonucleotide
acgggaggacattcaaaccaa

SEQ ID NO: 38 - DNA, 21 bp, Artificial Sequence
  Artificially synthesized oligonucleotide
atcttccatcgctgataccct

SEQ ID NO: 39 - DNA, 20 bp, Artificial Sequence
  Artificially synthesized oligonucleotide
acagcatagtaatctccgaa

SEQ ID NO: 40 - DNA, 20 bp, Artificial Sequence
  Artificially synthesized oligonucleotide
aagatgatagtaacgtgcaa

SEQ ID NO: 41 - DNA, 20 bp, Artificial Sequence
  Artificially synthesized oligonucleotide
gccaacgctaccaccatccg

SEQ ID NO: 42 - DNA, 21 bp, Artificial Sequence
  Artificially synthesized oligonucleotide
acgtgactcccaaggttagca

SEQ ID NO: 43 - DNA, 21 bp, Artificial Sequence
  Artificially synthesized oligonucleotide
ggctcatatcttagaatgcac

SEQ ID NO: 44 - DNA, 19 bp, Artificial Sequence
  Artificially synthesized oligonucleotide
gatctgtcgcaattaccac

SEQ ID NO: 45 - DNA, 20 bp, Artificial Sequence
  Artificially synthesized oligonucleotide
agcttccgagtatcagacct

SEQ ID NO: 46 - DNA, 21 bp, Artificial Sequence
  Artificially synthesized oligonucleotide
tgcactcttcttggcatagcg

SEQ ID NO: 47 - DNA, 19 bp, Artificial Sequence
  Artificially synthesized oligonucleotide
ccaacucaauucuucaatt

SEQ ID NO: 48 - DNA, 19 bp, Artificial Sequence
  Artificially synthesized oligonucleotide
acuauuacucugauacctt

SEQ ID NO: 49 - DNA, 19 bp, Artificial Sequence
  Artificially synthesized oligonucleotide
uguacuaaggucaaauctt

SEQ ID NO: 50 - DNA, 19 bp, Artificial Sequence
  Artificially synthesized oligonucleotide
uaaggcuaugaagagautt

SEQ ID NO: 51 - DNA, 19 bp, Artificial Sequence
  Artificially synthesized oligonucleotide
gcgagaagggaaguuuatt

SEQ ID NO: 52 - DNA, 19 bp, Artificial Sequence
  Artificially synthesized oligonucleotide
auggaauaugaaaugaatt

SEQ ID NO: 53 - DNA, 19 bp, Artificial Sequence
  Artificially synthesized oligonucleotide
gagcaaagccuccaagatt

SEQ ID NO: 54 - DNA, 23 bp, Artificial Sequence
  Artificially synthesized oligonucleotide
aagacacaugcacacguaugutt

SEQ ID NO: 55 - DNA, 20 bp, Artificial Sequence
  Artificially synthesized oligonucleotide
ucaagaagccaaggauaatt

SEQ ID NO: 56 - DNA, 365 bp, Homo sapiens
gggccgggcgcggtggctcacgcctgtaatcccagcactttgggaggccgaggcgggcggatcacaaggtcaggagatcgagaccatcctggctaacacagtgaaaccccgtctctactaaaaatacaaaaaattagccgggagtggtgtcgggcgcctgtagtcccagctactcgtgaggctgaggcaggagaatggcgtgaacccgggaggtggtggtgtcgggcgcctgtagtcccagctactcgtgaggctgaggcaggagaatggcgtgaacccgggaggtggagcttgcagcgagccgagatcgcgccactgcactccagcctgggtgacagagcgagactctgtctctttaaaggtac

SEQ ID NO: 57 - protein, 1275 aa, Homo sapiens [sequence omitted]

(19) United States
(12) Patent Application Publication
(10) Pub. No.: US 2021/0348164 A1
(43) Pub. Date: Nov. 11, 2021

(54) ENDOGENOUS CYTOPLASMIC ALU COMPLEMENTARY DNA IN AGE-RELATED MACULAR DEGENERATION

(71) Applicants: University of Virginia Patent Foundation, Charlottesville, VA (US); University of Kentucky Research Foundation, Lexington, KY (US)

(72) Inventors: Jayakrishna Ambati, Charlottesville, VA (US); Bradley David Unti Gelfand, Charlottesville, VA (US); Nagaraj Kerur, Crozet, VA (US); Shinichi Fukuda, Charlottesville, VA (US); Kameshwari Ambati, Charlottesville, VA (US); Benjamin Fowler, Miami, FL (US)

(73) Assignees: University of Virginia Patent Foundation, Charlottesville, VA (US); University of Kentucky Research Foundation, Lexington, KY (US)

(21) Appl. No.: 17/283,626

(22) PCT Filed: Oct. 9, 2019
(86) PCT No.: PCT/US2019/055413

§ 371 Date: Apr. 8, 2021

Related U.S. Application Data
(60) Provisional application No. 62/743,001, filed on Oct. 9, 2018.

(51) Int. Cl. C12N 15/113; A61K 47/54; A61K 31/536; A61K 31/496; C07K 16/18; A61P 27/02

(52) U.S. Cl. CPC C12N 15/113; A61K 47/543; A61P 27/02; A61K 31/496; C07K 16/18; A61K 31/536

(57) ABSTRACT

Provided are method for treating age-related macular degeneration (AGE), and/or preventing the occurrence or progression thereof in a subject in need thereof. In some embodiments, the methods include administering to the subject in need thereof a composition that has an effective amount of an inhibitor of reverse transcriptase (RTase) activity. Also provided are methods for protecting retinal pigmented epithelium (RPE) cells, retinal photoreceptor cells, and/or choroidal cells; methods for treating geographic atrophy of the eye, and/or for preventing occurrence or progression thereof; and pharmaceutical compositions for treating AGE and/or GA and/or for preventing the occurrence or progression thereof; and/or for protecting RPE cells, retinal photoreceptor cells, and/or choroidal cells.

### CROSS REFERENCE TO RELATED APPLICATION

The presently disclosed subject matter claims the benefit of U.S. Provisional Patent Application Ser. No. 62/743,001, filed Oct. 9, 2018; the disclosure of which is incorporated herein by reference in its entirety.

### GOVERNMENT INTEREST

This invention was made with government support under grants numbers GM114862, EY024336, RO1 EY022238, RO1 EY024068, and RO1 EY028027 awarded by the United States National Institutes of Health. The government has certain rights in the invention.

### TECHNICAL FIELD

The presently disclosed subject matter relates, in general, to the field of eye disorders, particularly age-related macular degeneration (AMD) and associated conditions. More particularly, the presently disclosed subject matter relates to methods for treating AMD or preventing the occurrence or progression thereof, and for protecting retinal pigmented epithelium (RPE) cells, retinal photoreceptor cells, and/or choroidal cells from damage related to accumulation of Alu reverse transcription products.

### BACKGROUND

Reverse transcription of RNA into DNA by retroviruses (Baltimore et al., 1970; Temin & Mizutani, 1970) is the cardinal exception to the “central dogma of molecular biology”: unidirectional flow of genetic information from DNA to RNA to proteins (Baltimore et al., 1970; Crick, 1970; Temin & Mizutani, 1970). Reverse transcription also occurs in eukaryotes in telomere synthesis and in the life cycle of retrotransposons, genetic elements that reproduce using host reverse transcriptase machinery via a copy-and-paste mechanism. Such endogenous retroelements have invaded the human genome and multiplied to occupy an astounding 42% of human DNA (Kazazian et al., 2017). However, the acquisition of new genetic material via reverse transcription is inefficient (Chun et al., 1997). The fate of cDNA generated from endogenous RNA that does not become integrated in the genome is poorly understood. Further, nearly all the biological activity of these reverse copies of host-derived genetic information has been considered in the context of whether they ultimately integrate in the genome.

AMD is a blinding disease that affects nearly 200 million people worldwide (Wong et al., 2014). The majority of patients are afflicted with the atrophic form of AMD, for which there are no effective therapies (Ambati et al., 2003). The accumulation of toxic retrotransposon Alu RNA in the RPE is involved in the pathogenesis of GA, an untreatable late stage of atrophic AMD (Kaneko et al., 2011; Dridi et al., 2012). The life cycle of Alu RNA involves reverse transcription and integration into the genome (Deininger & Batzer, 2002; Deininger, 2011).

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for treating age-related macular degeneration (AGE), and/or preventing the occurrence or progression thereof. In some embodiments, the presently disclosed methods comprise administering to a subject in need thereof a composition comprising an effective amount of an inhibitor of reverse transcriptase (RTase) activity. In some embodiments, the RTase activity is cytoplasmic RTase activity. In some embodiments, the inhibitor reduces cytoplasmic accumulation of a reverse transcription product of an Alu nucleic acid, optionally wherein the reverse transcription product is a single-stranded Alu cDNA. In some embodiments, the inhibitor of RTase activity is an inhibitor of an L1 ORF2 polypeptide RTase activity. In some embodiments, the inhibitor of RTase activity is selected from the group consisting of an L1 ORF2 inhibitor, a nucleoside reverse transcriptase inhibitor (NRTI), an alkylated derivative of an NRTI, and a non-nucleoside reverse transcriptase inhibitor (NNRTI). In some embodiments, the L1 ORF2 inhibitor is selected from the group consisting of an inhibitory nucleic acid that targets an L1 ORF2 transcription product and an antibody that is specific for an L1 ORF2 polypeptide. In some embodiments, the L1 ORF2 polypeptide is a human L1 ORF2 polypeptide, which in some embodiments comprises an amino acid sequence as set forth in SEQ ID NO: 57. In some embodiments, the NNRTI is selected from the group consisting of efavirenz (EFV) and delaviridine (DLV).

In some embodiments of the presently disclosed methods, the composition is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration; or intravenous administration. In some embodiments, the composition comprises an effective amount of a cell-permeable, non-immunogenic cholesterol-conjugated siRNA that targets an L1 ORF2-encoding nucleic acid.

In some embodiments, the presently disclosed subject matter also relates to methods for protecting retinal pigmented epithelium (RPE) cells, retinal photoreceptor cells, and/or choroidal cells in a subject in need thereof. In some embodiments, the presently disclosed methods comprise administering to the subject in need thereof a composition comprising an effective amount of an inhibitor of reverse transcriptase (RTase) activity. In some embodiments, the RTase activity is cytoplasmic RTase activity. In some embodiments, the inhibitor reduces cytoplasmic accumulation of a reverse transcription product of an Alu nucleic acid, optionally wherein the reverse transcription product is a single-stranded Alu cDNA. In some embodiments, the inhibitor of RTase activity is an inhibitor of an L1 ORF2 polypeptide RTase activity. In some embodiments, the inhibitor of RTase activity is selected from the group consisting of an L1 ORF2 inhibitor, a nucleoside reverse transcriptase inhibitor (NRTI), an alkylated derivative of an NRTI, and a non-nucleoside reverse transcriptase inhibitor (NNRTI). In some embodiments, the L1 ORF2 inhibitor is selected from the group consisting of an inhibitory nucleic acid that targets an L1 ORF2 transcription product and an antibody that is specific for an L1 ORF2 polypeptide. In some embodiments, the L1 ORF2 polypeptide is a human L1 ORF2 polypeptide, which in some embodiments comprises an amino acid sequence as set forth in SEQ ID NO: 57. In some embodiments, the NNRTI is selected from the group consisting of efavirenz (EFV) and delaviridine (DLV). In some embodiments, the composition is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration; or intravenous administration. In some embodiments, the composition comprises an effective amount of a cell-permeable, non-immunogenic cholesterol-conjugated siRNA that targets an L1 ORF2-encoding nucleic acid.

The presently disclosed subject matter also relates in some embodiments to methods for treating geographic atrophy (GA) of the eye, and/or preventing occurrence and/or progression thereof in a subject in need thereof. In some embodiments, the methods comprise administering to the subject in need thereof a composition comprising an effective amount of an inhibitor of reverse transcriptase (RTase) activity. In some embodiments, the RTase activity is cytoplasmic RTase activity. In some embodiments, the inhibitor reduces cytoplasmic accumulation of a reverse transcription product of an Alu nucleic acid, optionally wherein the reverse transcription product is a single-stranded Alu cDNA. In some embodiments, the inhibitor of RTase activity is an inhibitor of an L1 ORF2 polypeptide RTase activity. In some embodiments, the inhibitor of RTase activity is selected from the group consisting of an L1 ORF2 inhibitor, a nucleoside reverse transcriptase inhibitor (NRTI), an alkylated derivative of an NRTI, and a non-nucleoside reverse transcriptase inhibitor (NNRTI). In some embodiments, the L1 ORF2 inhibitor is selected from the group consisting of an inhibitory nucleic acid that targets an L1 ORF2 transcription product and an antibody that is specific for an L1 ORF2 polypeptide. In some embodiments, the L1 ORF2 polypeptide is a human L1 ORF2 polypeptide, which in some embodiments comprises an amino acid sequence as set forth in SEQ ID NO: 57. In some embodiments, the NNRTI is selected from the group consisting of efavirenz (EFV) and delaviridine (DLV). In some embodiments, the composition is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration; or intravenous administration. In some embodiments, the composition comprises an effective amount of a cell-permeable, non-immunogenic cholesterol-conjugated siRNA that targets an L1 ORF2-encoding nucleic acid.

The presently disclosed subject matter also relates in some embodiments to pharmaceutical compositions for treating age-related macular degeneration (AGE) and/or geographic atrophy (GA) of the eye, and/or preventing the occurrence or progression thereof, and/or for protecting a retinal pigmented epithelium (RPE) cell, a retinal photoreceptor cell, and/or a choroidal cell, the pharmaceutical composition comprising an effective amount of an inhibitor of reverse transcriptase (RTase) activity. In some embodiments, the RTase activity is cytoplasmic RTase activity. In some embodiments, the inhibitor reduces cytoplasmic accumulation of a reverse transcription product of an Alu nucleic acid, optionally wherein the reverse transcription product is a single-stranded Alu cDNA. In some embodiments, the inhibitor of RTase activity is an inhibitor of an L1 ORF2 polypeptide RTase activity. In some embodiments, the inhibitor of RTase activity is selected from the group consisting of an L1 ORF2 inhibitor, a nucleoside reverse transcriptase inhibitor (NRTI), an alkylated derivative of an NRTI, and a non-nucleoside reverse transcriptase inhibitor (NNRTI). In some embodiments, the L1 ORF2 inhibitor is selected from the group consisting of an inhibitory nucleic acid that targets an L1 ORF2 transcription product and an antibody that is specific for an L1 ORF2 polypeptide. In some embodiments, the L1 ORF2 polypeptide is a human L1 ORF2 polypeptide, which in some embodiments comprises an amino acid sequence as set forth in SEQ ID NO: 57. In some embodiments, the NNRTI is selected from the group consisting of efavirenz (EFV) and delaviridine (DLV). In some embodiments, the composition is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration; or intravenous administration. In some embodiments, the composition comprises an effective amount of a cell-permeable, non-immunogenic cholesterol-conjugated siRNA that targets an L1 ORF2-encoding nucleic acid. In some embodiments, the siRNA comprises a nucleotide sequence as set forth in any of SEQ ID NOs: 47-49 and 51-54.

Accordingly, it is an object of the presently disclosed subject matter to provide methods and compositions for treating AMD and/or for preventing the occurrence or progression thereof, and for protecting retinal pigmented epithelium (RPE) cells, retinal photoreceptor cells, and/or choroidal cells from damage related to accumulation of Alu reverse transcription products.

An object of the presently disclosed subject matter having been stated hereinabove, and which is achieved in whole or in part by the presently disclosed subject matter, other objects will become evident as the description proceeds when taken in connection with the accompanying drawings as best described herein below.

### BRIEF DESCRIPTION OF THE FIGURES

FIGS. 1A-1H. Alu cDNA accumulation in RPE of human GA eyes. FIG. 1A: Alu RNA or PBS subretinal injection into WT mice with LINE-1 (L1) siRNA or control siRNA. L1 siRNA blocked Alu RNA induced RPE degeneration. In fundus photographs (upper row), the degenerated retinal area is outlined by white arrowheads. RPE cellular boundaries are visualized by immunostaining flat mounts (bottom row) with zonula occludens-1 (ZO-1; red in color and gray in black and white) antibody. Loss of regular hexagonal cellular boundaries represents degenerated RPE. Scale bars, 10 μm. n=6-11. Binary and morphometric quantification of RPE degeneration are shown (*P<0.05; **P<0.01; ***P<0.001). PM, polymegethism (mean (SEM)). FIG. 1B: Alu RNA-induced RPE degeneration in WT mice is blocked by high doses (500 μmol) of efavirenz (EFV) and delaviridine (DLV), but not by nevirapine (NVP). n=6. FIG. 1C: Both Alu RNA and Alu with a G25C mutation in the left arm monomer (Alu G25C RNA) induced RPE degeneration in WT mice. n=6. FIGS. 1D and 1E: Photographs of normal human donor eye retina illustrating peripheral and peri-central areas (FIG. 1D) and geographic atrophy (GA) retina illustrating peripheral and junctional zone center (JZC) areas (FIG. 1E). Scale bars, 1 mm. FIGS. 1F and 1G: In situ hybridization of RPE whole mounts showing an absence of Alu cDNA in peripheral and pericentral areas of normal eyes (FIG. 1F), an abundance of Alu cDNA in the JZC and a paucity in peripheral areas of GA eyes (FIG. 1G). Insets show higher magnification. Red in color and darker gray in black and white, Alu cDNA; green in color and lighter gray in black and white, autofluorescence. Scale bars, 10 μm. FIG. 1H: Equator blotting of Alu RNA and Alu cDNA in macular (Mac) and peripheral (Peri) RPE of human GA (n=7) and normal (n=4) eyes. Densitometry of the bands corresponding to Alu RNA and Alu cDNA normalized to loading control (U6) and to the mean densitometry values for macular RPE of normal eyes.

FIGS. 2A-2K. Reverse transcriptase inhibition prevented Alu RNA toxicity. FIG. 2A: Immunoblots of LINE-1 (L1) in F9 mouse embryonal carcinoma cells transfected with various L1 siRNA sequences (mL1 3932siRNA (SEQ ID NO: 47), mL1 2672siRNA (SEQ ID NO: 48), or a Control (Luc siRNA) (SEQ ID NO: 50). FIG. 2B: Expression levels of genes with the greatest NCBI-BLAST sequence matches to the mouse L1 siRNA sequence was determined in human primary RPE cells transfected with L1 siRNA or luciferase (control) siRNA by quantitative real-time PCR and normalized to 18S rRNA levels. *P<0.05 by Mann-Whitney U test. Error bars show SEM. n=3. FIG. 2C: Alu RNA-induced RPE degeneration in wild-type (WT) mice (fundus photographs, left; ZO-1-stained (red in color/gray in black and white) flat mounts, right) was inhibited by low (25 μmol) doses (top row) of 3TC but not of EFV, and DLV, or NVP; and by high (500 μmol) doses (bottom row) of 3TC, efavirenz (EFV), and delavirdine (DLV), but not of nevirapine (NVP). Scale bars, 10 μm. n=6. FIG. 2D: Immunoblots showed caspase-1 activation in primary mouse bone marrow-derived macrophages (BMDM) treated with lipopolysaccharide (LPS) and ATP, and reduction thereof by 3TC but not by EFV, DLV, or NVP. Densitometry of the bands corresponding to caspase-1 normalized to loading control ((3-actin). FIG. 2E: Secondary structure scheme of an Alu RNA left arm mutation. Positions in the SRP9/14 binding site were mutated from G to C. FIGS. 2F and 2G, Reduced retrotransposition activity with Alu containing a G25C mutation in the left arm monomer (Alu G25C RNA) compared with Alu RNA. *P<0.05 by Mann-Whitney U test. n=4. Error bars show SEM. FIG. 2H, Dose-ranging studies of Alu G25C RNA in WT mice. n=6-8. FIG. 2I, Dose-ranging studies of Alu RNA in WT mice. n=6-18. FIG. 2J, Alu G25C RNAinduced RPE degeneration in WT mice is prevented by 3TC. n=6. FIG. 2K, Alu G25C RNA or PBS subretinal injection into WT mice with L1 siRNA or control siRNA. L1 siRNA prevented Alu G25C RNA-induced RPE degeneration. n=6-11. Fundus photographs, top; flat mounts stained for ZO-1 (red in color/gray in black and white), bottom. Scale bars, 10 μm (FIGS. 2H-2K). Binary and morphometric quantification of RPE degeneration are shown (*P<0.05; **P<0.01; ***P<0.001). PM, polymegethism (mean (SEM)).

FIGS. 3A-3G. Alu cDNA accumulation in human GA RPE. FIGS. 3A and 3B: Ex vivo fundus photographs of normal human donor eye retina (FIG. 3A) and geographic atrophy (GA) retina (FIG. 3B). Scale bars, 1 mm. FIGS. 3C and 3D: In situ hybridization of RPE whole mounts showing a paucity of Alu cDNA in peripheral and peri-central areas of normal eyes (FIG. 3C) and an abundance of Alu cDNA in the border of the atrophic area and the junctional zone of GA eyes (FIG. 3D). A few scattered foci of Alu cDNA were present in the peripheral disease-free area in GA (FIG. 3D). Red in color/gray in black and white, Alu cDNA; Green in color/lighter gray in black and white, autofluorescence of RPE cells. Scale bars, 100 The junctional zone is a 500-μm annulus circumscribing the atrophic region. Atrophy border is the interface of the atrophic region and the junctional zone. FIG. 3E: Low magnification of whole mount in situ hybridization of Alu cDNA (red in color/gray in black and white) in the RPE of a GA eye showed enrichment in the atrophic border and junctional zone. Scale bars, 500 FIG. 3F: Whole mount in situ hybridization of Alu cDNA (red in color/gray in black and white) in the RPE of a GA eye showed loss of the signal following treatment with single-stranded specific 51 nuclease. Scale bars, 500 FIG. 3G: In situ hybridization of Alu cDNA (green in color/light gray in black and white) in primary human RPE cells transfected with artificially synthesized single-stranded Alu cDNA (ss Alu cDNA) with or without 51 nuclease. DAPI (blue in color/darker gray in black and white), Scale bars, 10 μm.

FIGS. 4A-4C. Alu cDNA absent in other retinal diseases. FIG. 4A: Ex vivo fundus photograph of an eye with RPE atrophy that developed subsequent to treatment of central retinal vein occlusion with anti-angiogenic drugs. In situ hybridization of RPE whole mounts showing no Alu cDNA in peripheral RPE or at the border of the atrophic RPE. Scale bars, 200 Red in color/gray in black and white, Alu cDNA; green in color/lighter gray in black and white, autofluorescence of RPE cells. FIG. 4B: Abundant Alu cDNA detected in the RPE of GA eyes but not in the RPE of eyes with Leber congenital amaurosis, Joubert syndrome, Stargardt macular dystrophy, or autosomal recessive retinitis pigmentosa. Red in color/gray in black and white, Alu cDNA; Green in color/lighter gray in black and white, RPE65; blue in color/gray in black and white, DAPI. Scale bars, 50 FIG. 4C: In situ hybridization shows no Alu cDNA formation in primary human RPE cells subjected to acid injury (hydrochloric acid; HCl, pH 4.0 medium) or osmotic stress (distilled H₂O). Alu cDNA (green in color/light gray dots in black and white), DAPI (blue in color/darker gray in black and white). Scale bars, 10 μm.

FIGS. 5A-5L. Reverse transcribed endogenous Alu cDNA originating from Alu RNA in human cells. FIG. 5A: The schema of the method (Alu c-PCR) used to purify and amplify reverse transcribed single-stranded DNA. Total cell lysate was fractionated into nuclear and cytoplasmic fractions, and then RNase-treated. Cytoplasmic DNA was tailed on the 3′ end to generate a 20-40 poly A tail by using terminal deoynucleotidyl transferase (TdT), and then the poly T-anchored primer (TAV oligo) was annealed to the poly A-tail of the template strand and extended. Anchored DNA was amplified using primer specific for the anchor and reverse primer specific for the sequence within Alu. In primary human RPE cells, Alu cDNA was decreased by 3TC treatment. *P <0.05 by Mann-Whitney U test. n=4. Error bars show SEM. FIG. 5B: Alu single-stranded DNA (ssDNA), but not Alu circular double-stranded DNA (dsDNA), is amplified by Alu c-PCR. n=3. FIG. 5C: Real-time RT-PCR for U6 RNA and tRNA confirmed proper enrichment and lack of crosscontamination in nuclear (Nuc) and cytoplasmic (Cyto) fractions. FIG. 5D: Direct real-time PCR using a primer set for the intron-intron junction of GPR15 showed absence of genomic DNA contamination in the cytoplasmic fraction. Error bars show SEM. N.D., not detected. FIG. 5E: Cytoplasmic and nuclear RNA isolated from primary human RPE cells, run on a 0.9% agarose gel, show that genomic DNA was present in the nuclear fraction but not detected in the cytoplasmic fractions. FIG. 5F: Immunoblotting for TBP-1 and tubulin confirmed proper enrichment and lack of crosscontamination in nuclear and cytoplasmic fractions. FIG. 5G: Endogenous Alu cDNA abundance in primary human RPE and ARPE-19 cells was reduced by the NRTIs 3TC, d4T, and a mixture thereof (cocktail), but not by trimethyl-3TC (TM-3TC). n=4. Error bars show SEM. FIGS. 5H-5J, TM-3TC did not inhibit L1 retrotransposition. FIG. 5H: Retrotransposition events in HeLa cells, assessed by the enhanced green fluorescent protein (EGFP) cell culture L1 retrotransposition flow cytometry assay, were reduced by treatment with 3TC but not TM-3TC. Cells were gated based on background fluorescence of plasmid JM111, which has two point mutations in L1-ORF1 that abolish retrotransposition. Data were normalized with RPS set to 1 (n=4). FIGS. 5I and 5J: HeLa cells were transduced with a GFP-expressing lentivirus in the presence or absence of 3TC (50 μM) or TM-3TC (50 μM) for 48 hours. Quantification (FIG. 5I) and representative images (FIG. 5J). Cells were stained with Hoechst (blue in color/gray in black and white). n=4-11. FIG. 5K: Alu cDNA blotting in primary human RPE cells treated with RNase, a double-stranded DNase, or a single-stranded DNase. FIG. 5L: Copy number of Alu cDNA per cell in primary human peripheral blood mononuclear cells, ARPE-19 cells, primary human RPE cells, and human embryonic kidney-293-T cells (HEK-T), human umbilical vein endothelial cells (HUVEC), primary human subcutaneous pre-adipocytes, Primary human epidermal keratinocytes, primary human dermal fibroblasts, umbilical artery vascular smooth muscle cells (SMCs), and primary human skeletal myoblasts. n=3-8. Error bars show SEM.

FIGS. 6A-6C. Alu cDNA subfamilies in the cytoplasmic fraction of primary human RPE cells. FIG. 6A: Distribution of uniquely and multi-mapped (all alignments mapping within same subfamily) Alu read counts per subfamily. Error bars show SEM. FIG. 6B: Alu expression in RPE-specific genes versus other genes. Distributions represent number of Alu reads mapped within 2,000 bp of each gene locus. FIG. 6C: List of single nucleotide variants in gene loci statistically associated with AMD within 2,000 bp of which Alu reads were identified.

FIGS. 7A-7G. Endogenous Alu cDNA synthesized via reverse transcription. FIGS. 7A and 7B: Equator blotting (FIG. 7A) and in situ hybridization (FIG. 7B) show increased cytoplasmic Alu cDNA in primary human RPE cells exposed to Alu RNA (compared to mock transfection), heat shock (compared to no heat), or DICER1 antisense oligonucleotides (DICER1 AS) (compared to control scrambled (Scr) AS), and a reduction following 3TC treatment. Blots of whole cell lysate (Alu RNA; U6) and cytoplasmic fraction (Alu cDNA) (FIG. 7A). Alu cDNA (green in color/gray dots in black and white), ZO-1 (red in color/darker gray in black and white), DAPI (blue in color/lighter gray in black and white). Scale bars, 10 μm (FIG. 7B). FIG. 7C: Adaptor-based PCR-based quantification (Alu c-PCR) of Alu cDNA in primary human RPE cells exposed to Alu RNA, heat shock, or DICER1 antisense oligonucleotides (DICER1 AS) shows 3TC reduced Alu cDNA induction. n=4. Error bars show SEM. *P<0.05 by one-way ANOVA with Bonferroni's post-hoc test. FIGS. 7D and 7E: In situ hybridization (FIG. 7D) and quantification (FIG. 7E) of ZO-1-stained (red in color/gray in black and white) RPE flat mounts shows increased Alu cDNA (green in color/lighter gray stippling in black and white) at 12 hours, 1 day and 4 days following subretinal Alu RNA in Casp1/4 dko mice, and reduced Alu cDNA levels in 3TC-treated mice. Scale bars, 10 μm. *P<0.05 by one-way ANOVA with Bonferroni's post-hoc test. n=6. Error bars denote SEM. FIGS. 7F and 7G: In situ hybridization shows Alu cDNA (green in color/lighter gray stippling in black and white) abundance in primary human RPE cells exposed to Alu RNA transfection, DICER1 AS, or heat shock is reduced by treatment with L1 siRNA compared to control siRNA (FIG. 7F), and is reduced by treatment with high doses of efavirenz (EFV) and delavirdine (DLV), but not nevirapine (NVP) (FIG. 7G). SiR (F-actin, red in color/darker gray in black and white), DAPI (blue in color/lighter gray in black and white). Scale bars, 10 μm.

FIGS. 8A-8G. Endogenous Alu cDNA in RPE cells. FIG. 8A: In situ hybridization of Alu cDNA (green in color/gray stippling in black and white) in primary human RPE cells transfected with Alu RNA. DAPI (blue in color/lighter gray in black and white), SiR (F-actin, red in color/darker gray in black and white). Scale bars, 10 Orthogonal views obtained by laser scanning confocal microscopy (far right image) showed co-localization of Alu cDNA (green in color/gray stippling in black and white) with cytoplasmic F-actin (SiR, red in color/darker gray in black and white) with DAPI counterstain. FIGS. 8B and 8C: In situ hybridization of Alu cDNA in primary human RPE cells exposed to DICER1 antisense oligonucleotides (DICER1 AS). At 12 hours after exposure of DICER1 AS, Alu cDNA (green in color/gray stippling in black and white) was localized in the cytoplasm. At 24 hours, Alu cDNA accumulation remained predominantly cytoplasmic but occasionally was observed in the nucleus. DAPI (blue in color/lighter gray in black and white), SiR (Factin, red in color/darker gray in black and white). Scale bars, 10 FIG. 8D: Equator blotting shows, following heat shock, endogenous Alu cDNA is heterogeneous in length. Synthesized single-stranded Alu cDNA (293-nt) is used as a control. FIG. 8E: In situ hybridization in primary human RPE cells showed that treatment with 51 nuclease eliminated the Alu cDNA (green in color/gray stippling in black and white) signal in cells exposed to Alu RNA, heat shock, or DICER1 AS. DAPI (blue in color/lighter gray in black and white), SiR (F-actin, red in color/darker gray in black and white). Scale bars, 10 FIGS. 8F and 8G: In situ hybridization of Alu cDNA in ARPE-19 cells showed that induction of Alu cDNA (green in color/gray stippling in black and white) by heat shock or DICER1 AS was reduced by 3TC but not TM-3TC. Treatment with single-stranded 51 nuclease eliminated the Alu cDNA signal. DAPI (blue in color/lighter gray in black and white), SiR (F-actin, red in color/darker gray in black and white). Scale bars, 10 μm.

FIGS. 9A and 9B. Endogenous cytoplasmic Alu cDNA induction and sequence. FIG. 9A: Treatment with Alu RNA, heat shock, or DICER1 AS yielded an increase in cytoplasmic Alu cDNA levels, as monitored by direct amplification of extracted cytoplasmic DNA by real-time PCR (without reverse transcription) and normalized by cell number. *P<0.05 by Mann-Whitney U test. Error bars show SEM. n=3-4. FIG. 9B: DNA extracted from Alu RNA-transfected mouse fibroblasts and then subjected to TA cloning and sequencing. The Alu element (SEQ ID NO: 56) is about 300 bases long and consists of two similar monomers: the left and right arms joined by an A-rich linker and followed by a poly(A) tail (Taylor et al., 2013). The left arm consists of RNA polymerase III binding sites (Box A and Box B). The right arm occasionally contains a terminal poly A tail. Artificially synthesized Alu sequence (Alu). Alignment of Alu cDNA isolated from mouse fibroblasts after Alu RNA transfection (Samples 1, 2, and 3). The sequences perfectly matched the reference Alu sequence of SEQ ID NO: 56.

FIGS. 10A-10I. Alu RNA toxicity in mice, L1 in GA, and L1 and NNRTIs in Alu cDNA formation. FIG. 10A: At 12 hours after Alu RNA subretinal injection, the RPE of WT mice appear normal. Mild RPE morphological changes appear 1 day after injection and frank RPE degeneration is evident by 2-3 days after injection. n=6. Binary and morphometric quantification of RPE degeneration are shown (*P<0.05; **P<0.01; ***P<0.001). PM, polymegethism (mean (SEM)). FIG. 10B: L1 ORF1 and ORF2 mRNA abundance, monitored by real-time PCR, are higher in the macular RPE of human GA eyes (n=8) compared with normal human eyes (n=5). *P<0.05, **P<0.01 by Mann-Whitney U test. Error bars show SEM. FIG. 10C: Representative immunoblots of macular RPE from individual human donor eyes showed that L1 ORF1p and ORF2p abundance, normalized to vinculin, was increased in GA eyes compared to control eyes. FIG. 10D: Immunoblot analysis of protein generated from expression plasmid containing codon optimized L1 (pLD401) transiently transfected in NIH3T3 Tet ON cells using anti-human L1 ORF1p antibody (top). Immunoblot analysis of nuclear or cytoplasmic extract from Ntera2D cells using anti-human L1 ORF2p antibody (middle). Immunoblot analysis of whole cell extract from F9 mouse embryonal carcinoma cell line or NIH-3T3 cells using anti-mouse L1 ORF2p antibody (bottom). TBP1, tubulin and β-actin are used as loading control. FIG. 10E: Immunoblots of L1 in primary human RPE cells transfected with various L1 siRNA sequences (hL1 1288siRNA (SEQ ID NO: 51), hL1 1264siRNA (SEQ ID NO: 52), hL1 1329 siRNA (SEQ ID NO: 53), or Control (Scr siRNA; SEQ ID NO: 55)). FIG. 10F: Expression levels of genes with the greatest NCBI-BLAST sequence matches to the human L1 siRNA sequence was determined in human primary RPE cells transfected with L1 siRNA or luciferase (control) siRNA by quantitative real-time PCR and normalized to 18S rRNA. n=3. FIG. 10G: Direct amplification by real-time PCR (without reverse transcription) of Alu cDNA in primary human RPE cells treated with Alu RNA, heat shock or DICER1 antisense oligonucleotides (DICER1 AS), showed that Alu cDNA induction was reduced by L1 siRNA compared with control siRNA. * P<0.05 by one-way ANOVA with Bonferroni's post-hoc test. n=4. Error bars show SEM. FIG. 10H: In situ hybridization of Alu cDNA (green in color/gray stippling in black and white) in ARPE-19 cells after heat shock with L1 siRNA. DAPI (blue in color/lighter gray in black and white), SiR (F-actin, red in color/darker gray in black and white). Scale bars, 10 FIG. 10I: RPE whole mount in situ hybridization for Alu cDNA using Casp1/4 dko mice. Efavirenz (EFV) and delavirdine (DLV), but not nevirapine (NVP), blocked endogenous cDNA synthesis. Alu cDNA (green in color/gray stippling in black and white), ZO-1 (red in color/darker gray in black and white). Scale bars, 10 Bar graph quantifies signal intensity. * P<0.05 by one-way ANOVA with Bonferroni's post-hoc test. Error bars show SEM. n=6.

FIGS. 11A-11I. Endogenous Alu cDNA induces RPE toxicity. FIG. 11A: Equator blotting shows production of Alu cDNA in mouse L fibroblasts following transfection of Alu RNA or Alu with a G25C mutation in the left arm monomer (Alu G25C RNA). FIG. 11B: In situ hybridization shows Alu cDNA (green in color/gray stippling in black and white) in mouse L cells after following transfection of Alu RNA or Alu G25C mutant RNA, and reduced Alu cDNA abundance following treatment with 3TC but not trimethyl-3TC (TM-3TC). SiR (F-actin, red in color/darker gray in black and white), DAPI (blue in color/lighter gray in black and white). Scale bars, 10 FIGS. 11C and 11D: In situ hybridization (FIG. 11C) and quantification (FIG. 11D) of ZO-1-stained (red in color/darker gray in black and white) RPE flat mounts shows increased Alu cDNA (green in color/gray stippling in black and white) following subretinal Alu RNA or Alu G25C RNA in Casp1/4 dko mice, and reduced Alu cDNA abundance following treatment with 3TC but not TM-3TC. Scale bars, 10 μm. *P<0.05 by one-way ANOVA with Bonferroni's post-hoc test. n=6. Error bars denote SEM. FIGS. 11E and 11F: In situ hybridization shows Alu cDNA (green in color/gray stippling in black and white) production in Alu RNA-treated RPE cells of Rattus norvegicus (WT) rat (FIG. 11E) but not of Oryzomys palustris (FIG. 11F). FIG. 11G: Alu cDNA formation, monitored by direct amplification by real-time PCR without reverse transcription, is reduced in Oryzomys palustris RPE cells treated with Alu RNA compared with Rattus norvegicus (WT rat) RPE cells. *P<0.05 by Mann-Whitney U test. Error bars show SEM. n=4. FIGS. 11H and 11I: Alu RNA induced RPE degeneration in WT rat eyes (FIG. 11H) but not Oryzomys palustris (FIG. 11I), whereas Alu cDNA induced RPE degeneration in both species. SiR (F-actin, red in color/darker gray in black and white), DAPI (blue in color/lighter gray in black and white). Scale bars, 10 μm. Binary and morphometric quantification of RPE degeneration are shown (*P<0.05, **P<0.01, ***P<0.001). PM, polymegethism (mean (SEM)).

FIGS. 12A-12D. Alu RNA and Alu cDNA toxicity. FIG. 12A: Multiple Alu family cDNAs induced RPE degeneration in WT mice. n=6. FIG. 12B: Dose-ranging studies showed that Alu RNA (FIG. 7D) was less potent than Alu cDNA (FIG. 12B) at inducing RPE degeneration in WT mice. n=6-18. Binary and morphometric quantification of RPE degeneration are shown (*P<0.05, **P<0.01, ***P<0.001). PM, polymegethism (mean (SEM)). FIGS. 12C and 12D: Reverse sequence of Alu cDNA (FIG. 12C) or a DNA sequence complementary to 7SL RNA (FIG. 12D) do not induce RPE degeneration in WT mice. n=6. Binary and morphometric quantification of RPE degeneration are shown (*P<0.05; **P<0.01; ***P<0.001). PM, polymegethism (mean (SEM)).

FIGS. 13A-13O. L1 in Oryzomys, nuclear- or cytoplasmic-targeted NRTI; Colocalization of L1 ORF2p and Alu, and self-priming activity of Alu. FIG. 13A: L1 ORF1 and ORF2 mRNA abundance, monitored by real-time PCR, are higher in WT rat RPE cells compared with Oryzomys palustris RPE cells. ** P<0.01 by Mann-Whitney U test. Error bars show SEM. n=4. FIG. 13B: Real-time PCR analysis of L1 ORF1 and ORF2 mRNA from Oryzomys palustris RPE cells transfected with plasmid expressing rat L1 ORF1 and ORF2. ** P<0.01 by Mann-Whitney U test. Error bars show SEM. n=4.

FIG. 13C: Retrotransposition assays show that in vitro enforced expression of an endonuclease-deficient (EN-) L1 ORF2 or a reverse transcriptase-deficient (RT-) mutant could not support retrotransposition of Alu RNA. **P<0.01, by one-way ANOVA with Bonferroni's post-hoc test. n=3. FIGS. 13D and 13E: Retrotransposition assays show that 3TC and nuclear targeted 3TC (Cpep-3TC) (FIG. 13D), but not cytoplasmic targeted 3TC (PA-4-3TC) (FIG. 13E), blocked retrotransposition of Alu RNA. n=3. FIG. 13F: Cytoplasmic fractions of RNaseH-deficient HeLa cells co-expressing V5-tagged L1 ORF2 (V5-ORF2) and Alu RNA (either biotinylated or unlabeled) were subjected to streptavidin pull-down. V5 immunoblots in the input and pull-down samples show similar V5-ORF2p expression in both Alu RNA-transfected samples, and specific interaction upon pull-down in the biotinylated Alu RNA-treated sample. FIG. 13G: Immunoblots of V5-ORF2 confirm cytoplasmic localization of V5-ORF2p (top). Immunoblots of tubulin confirm purity of subcellular fractions (bottom). FIG. 13H: Cytoplasmic fractions of cells co-expressing biotinylated Alu RNA and either V5-ORF2 or V5-empty plasmid were subjected to anti-V5-immunoprecipitation. The top panel shows detection of biotinylated Alu RNA in the cytoplasmic fraction. The bottom panel shows detection of V5-ORF2p. FIG. 13I: Equator blotting shows detection of Alu cDNA following anti-V5-immunoprecipitation in cells co-expressing biotinylated Alu RNA and either V5-ORF2 or V5-empty plasmid. The migration of an in vitro synthesized Alu cDNA is shown for comparison. FIG. 13J: Fluorescence imaging of Oryzomys palustris RPE cells co-expressing V5-ORF2 and fluorescein-labeled Alu RNA (transfected 48 hours after V5-ORF2 transfection) displays diffuse localization of V5-ORF2p (red in color/darker gray in black and white) and punctate foci of fluorescein-Alu RNA (green in color/lighter gray stippling in black and white) at 2 hours after Alu RNA transfection. Cytoplasmic co-localization of multiple punctate foci of fluorescein-Alu RNA with V5-ORF2p seen at 8 hours after Alu RNA transfection. V5-ORFp localization remains diffuse throughout the cell in the absence of fluorescein-Alu RNA transfection (Mock). From left to right, the columns showed merged green & red channel, green channel, and red channel images. FIG. 13K: In situ hybridization of Oryzomys palustris RPE cells co-expressing V5-ORF2 and fluorescein-labeled Alu RNA (transfected 48 hours after V5-ORF2 transfection) shows cytoplasmic co-localization of V5-ORF2p (red in color/darker gray in black and white) and Alu cDNA (teal in color/lighter gray stippling in black and white) at 8 hours after Alu RNA transfection. DAPI (blue in color/lighter gray in black and white). Scale bars, 10 FIG. 13L: In situ hybridization shows Alu cDNA (green in color/lighter gray stippling in black and white) formation in WT mouse RPE cells following transfection of uncapped Alu RNA but not of Alu RNAs capped on the 3′ end with the chain terminators dideoxy thymidine base (ddTTP) or cordycepin triphosphate. DAPI (blue in color/lighter gray in black and white). Scale bars, 10 FIG. 13M: Alu cDNA abundance in the cytoplasmic fraction of WT mouse RPE cells, monitored by direct reverse transcriptase assay in the absence of external primers, followed by Alu specific real-time RT-PCR, was greater following transfection with uncapped Alu RNA compared with Alu RNA 3′ capped with ddTTP or cordycepin triphosphate. Error bars show SEM. *P<0.05. FIG. 13N: Equator blotting shows uncapped Alu RNA, compared to 3′ cordycepin triphosphate-capped Alu RNA, supports more formation of Alu cDNA by direct reverse transcriptase assay using cytoplasmic fraction of WT mouse RPE cells in the absence of external primers. Synthesized single-stranded Alu cDNA (293-nt) is used as a control. FIG. 13O: Subretinal injection of Alu RNAs capped on the 3′ end with the chain terminators dideoxy thymidine base (ddTTP) or cordycepin triphosphate do not induce RPE degeneration in wild-type (WT) mice, whereas uncapped Alu RNA induced RPE degeneration (fundus photographs, left; ZO-1-stained (red in color/darker gray in black and white) flat mounts, right). Scale bars, 10 n=6. Scale bars, 10 Binary and morphometric quantification of RPE degeneration are shown (*P<0.05; **P<0.01; ***P <0.001). PM, polymegethism (mean (SEM)).

FIGS. 14A-14D. L1 ORF2 supports Alu cDNA formation and RPE degeneration. FIG. 14A: Alu RNA induced RPE degeneration in Oryzomys palustris following enforced subretinal in vivo expression of L1 ORF2p but not of L1 ORF1p. FIG. 14B: Alu RNA-induced RPE degeneration in L1 ORF2-expressing Oryzomys palustris is blocked by high dose delaviridine (DLV; 500 μmol). FIG. 14C: Formation of Alu cDNA following Alu RNA transfection of Oryzomys RPE cells, monitored by Alu-specific qPCR of cytoplasmic fractions, showed greater reverse transcriptase activity in cells with enforced expression of L1 ORF2 compared to L1 ORF1; this was inhibited by efavirenz (EFV). Alu cDNA formation was greater following expression of endonuclease-deficient (EN-) L1 ORF2 mutant compared to expression of a reverse transcriptase-deficient (RT-) L1 ORF2 mutant. **P<0.01 by one-way ANOVA with Bonferroni's post-hoc test. n=4-6. Error bars denote SEM. FIG. 14D: Alu RNA induces RPE degeneration in Oryzomys following in vivo enforced expression of L1 ORF2 (EN-) but not of following L1 ORF2 (RT-). Scale bars, 10 Binary and morphometric quantification of RPE degeneration are shown (*P<0.05, **P<0.01, ***P<0.001). PM, polymegethism (mean (SEM)).

FIGS. 15A-15K. Endogenous Alu cDNA synthesis and RPE toxicity signalling mechanism. FIG. 15A: Immunoblots show caspase-1 activation in primary human RPE cells treated with Alu RNA or Alu cDNA. FIGS. 15B and 15C: Alu cDNA does not induce RPE degeneration in Mb21d1^{-/-} (FIG. 15B) or Nlrp3^{-/-} (FIG. 15C) mice. n=6. FIGS. 15D and 15E: Alu cDNA-induced RPE degeneration in WT mice is blocked by 3TC (500 μmol) (FIG. 15D) and by TM-3TC but not high doses (500 μmol) of efavirenz (EFV), delavirdine (DLV), or nevirapine (NVP) (FIG. 15E), or by PBS (d). n=6. FIGS. 15F and 15G: Alu cDNA (green in color/lighter gray stippling in black and white) synthesis in primary human RPE cells exposed to Alu RNA, monitored by in situ hybridization (top; SiR (F-actin, red in color/darker gray in black and white), DAPI (blue in color/lighter gray in black and white)) and Alu RNA-induced RPE degeneration in WT mice (bottom) were not blocked by Cpep-3TC (nuclear-targeting cyclic peptide-conjugated 3TC) or control Cpep (FIG. 15F) but were blocked by PA-4-3TC (cytoplasmic-targeting NRTI formulation) (FIG. 15G). Scale bars, 10 μm. n=6. Binary and morphometric quantification of RPE degeneration are shown (*P<0.05, **P<0.01, ***P<0.001). PM, polymegethism (mean (SEM)). FIGS. 15H-15K: Reverse transcriptase activity, monitored by Alu specific qPCR, of nuclear or cytoplasmic fractions of mouse embryonal carcinoma cell line (F9) is evaluated by Alu cDNA formation from Alu RNA, and showed greater RT activity in cytoplasmic compared to nuclear fractions (FIG. 15H). Heat treatment of cytoplasmic fractions abolished Alu cDNA formation (FIG. 15I). n=7. Cytoplasmic fractions of L1 siRNA-treated cells showed reduced Alu cDNA formation compared to control siRNA-treated cells (FIG. 15J). Treatment of cytoplasmic fractions with AZT-triphosphate (AZT-TP) but not diethyl-AZT (DE-AZT) reduced Alu cDNA formation (FIG. 15K). *P<0.05, **P<0.01, ***P<0.001 by Mann-Whitney U test or one-way ANOVA with Bonferroni's post-hoc test. n=3-8. Error bars denote SEM.

### DETAILED DESCRIPTION

The Alu mobile genetic element propagates through retrotransposition by hijacking LINE-1 (L1) reverse transcriptase and endonuclease enzymatic activities (Feng et al., 1996; Moran et al., 1996; Dewannieux et al., 2003), and occupies 11% of the human genome (Dewannieux et al., 2003; Venter et al., 2001). Reverse transcription of Alu RNA is presumed to occur only in the nucleus, concurrent with genomic integration1. However, whether reverse transcriptase-derived Alu complementary DNA (cDNA) is synthesized independently of genomic integration is unknown. Excess Alu RNA in geographic atrophy (GA; Kaneko et al., 2011), an untreatable advanced form of age-related macular degeneration (AMD), triggers retinal pigmented epithelium (RPE) death via inflammatory pathways (Tarallo et al., 2012; Kerur et al., 2018). Nucleoside reverse transcriptase inhibitors (NRTI), due to an intrinsic anti-inflammatory activity, block RPE degeneration even when stripped of their reverse transcriptase-inhibitory ability (Fowler et al., 2014); thus, the role of reverse transcriptase in Alu RNA toxicity is unclear.

Disclosed herein is the discovery that Alu RNA-induced RPE degeneration and inflammation are mediated via cytoplasmic L1-reverse transcribed Alu cDNA independently of retrotransposition, and that Alu cDNA levels are increased in the RPE of humans with GA. In a rodent lacking L1 activity, Oryzomys palustris (Casavant et al., 2000; Grahn et al., 2005; Rinehart et al., 2005; Yang et al., 2014), Alu RNA did not induce robust Alu cDNA production or RPE degeneration. In two large patient health record databases, exposure to NRTIs was associated with reduced risk of developing atrophic AMD, thus identifying inhibitors of this new Alu lifecycle shunt as potential therapies for a major cause of blindness. We also detected reverse transcriptase-derived Alu cDNA in the cytoplasm of numerous human cell types; thus, Alu cDNA might be relevant in other diseases that display Alu RNA accumulation and inflammation (Kahlenberg et al., 2011; Masters et al., 2011; Yan et al., 2013; Italiani et al., 2014; Hung et al., 2015; Johann et al., 2015; Prudencio et al., 2017). The discovery of a pathogenic endogenous human cDNA shows that the threat posed by L1 to human health is not confined to mutagenic retrotransposition and should prompt a search for cellular centurions that combat reverse transcription. We speculate that Alu and other endogenous cytoplasmic cDNAs could be a novel class of gene regulators and also might influence the effects of L1 on speciation and genetic diversity.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Furthermore, the terms first, second, third, and the like as used herein are employed for distinguishing between similar elements and not necessarily for describing a sequential or chronological order. It is to be understood that the terms so used are interchangeable under appropriate circumstances and that the subject matter described herein is capable of operation in other sequences than described or illustrated herein.

Following long-standing patent law convention, the articles “a”, “an”, and “the” refer to “one or more” when used in this application, including in the claims. For example, the phrase “a cell” refers to one or more cells. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising and active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains the active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

As used herein, the term “isolated” when referring to cells or a cell population refers to cells or a cell population collected from a subject, in some embodiments a mammalian subject, and in some embodiments a human. Typically, collection of the desired cells or cell population is achieved based on detection of one or more cell markers, such as but not limited to antibody-based detection.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the L1 ORF2 gene, an exemplary gene product of which is disclosed in Accession No. AH002566.2 of the GENBANK® biosequence database and which encodes SEQ ID NO: 57 (also disclosed as Accession No., the sequences disclosed herein are intended to encompass homologous genes and gene products from other animals including, but not limited to other mammals.

The methods of the presently disclosed subject matter are particularly useful for warm-blooded vertebrates. Thus, in some embodiments the presently disclosed subject matter concerns mammals.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

For some markers, expression or absence of expression is often in fact based on comparison with other cells which also express the marker. For these markers determining positive or negative expression is based on a threshold. Methods for determining positive or negative expression based on thresholds are known to the person skilled in the art and typically involve calibrating based on a “negative control”. Accordingly, it will be understood that for these markers, reference to positive expression in fact implies “elevated expression compared to negative controls” and “negative expression” in fact refers to “reduced expression compared to positive controls”.

When referring to a cell population, reference is made to a population which “expresses gene X” where at least 10%, 20%, or 30% or 40%, 50%, or 60% or 70%, 80%, or 90% or 95%, 96%, 97%, 98%, 99%, or even 100% of the cells within the population express the gene of interest. By “substantially free” is intended less than about 25%, 20%, 15%, 10%, 9%, 8%, 7%, 6%, 5%, 4%, 3%, 2%, 1%, or even 0% of the cells in the population express the gene of interest.

### II. Methods of the Presently Disclosed Subject Matter

Disclosed herein is the discovery of the existence of reverse transcribed Alu cDNA in the cytoplasm of numerous human cell types, the accumulation of Alu cDNA in human eyes with geographic atrophy (GA), and the induction of RPE degeneration in vivo by Alu cDNA. Evidence is also presented from two independent patient health records databases of a protective association of nucleoside reverse transcriptase inhibitors (NRTIs) with atrophic AMD, which suggests that clinically approved drugs potentially could be repurposed for this disease.

Thus, in some embodiments the presently disclosed subject matter relates to methods for treating age-related macular degeneration (AGE), or preventing the occurrence or progression thereof, in a subject in need thereof. In some embodiments, the presently disclosed subject matter relates methods for protecting a retinal pigmented epithelium (RPE) cell, a retinal photoreceptor cell, or a choroidal cell. In some embodiments, the presently disclosed subject matter relates to methods for treating geographic atrophy (GA) of the eye, or preventing occurrence or progression thereof, the method comprising administering to a subject in need thereof a composition comprising an effective amount of an inhibitor of reverse transcriptase (RTase) activity.

In some embodiments of the presently disclosed methods, the presently disclosed methods comprise administering to a subject in need thereof a composition comprising an effective amount of an inhibitor of reverse transcriptase (RTase) activity, which in some embodiments is cytoplasmic RTase activity. As used herein, the phrase “inhibitor of reverse transcriptase (RTase) activity” refers to any molecule that directly or indirectly inhibits the biological activity of an RTase. In some embodiments, the inhibitor reduces cytoplasmic accumulation of a reverse transcription product of an Alu nucleic acid, optionally wherein the reverse transcription product is a single-stranded Alu cDNA.

In some embodiments, the inhibitor of RTase activity is an inhibitor of an L1 ORF2 polypeptide RTase activity. The phrase “L1 ORF2 polypeptide” refers to a Long Interspersed Element-1 ORF2 encoded protein (L1 ORF2p). LINE-1 (Long Interspersed Elements, L1) elements are the largest family of human retrotransposons, which are mobile genetic elements spreading in the human genome via RNA intermediates that are reverse transcribed in cDNA copies inserted into the genome. Each functional L1 copy contains two open reading frames—ORF1 and ORF2—that are expressed as a bicistronic RNA. ORF1 and ORF2 encode a 40 kiloDalton (kDa) RNA-binding protein (ORF1p) and a 150 kDa polyprotein (ORF2p), respectively. ORF2p includes an N-terminal endonuclease domain and an adjacent reverse transcriptase (RT) domain (Mathias et al., 1991). Therefore, RT is expressed as part of the L1-ORF2 polyprotein. Notably, L1-encoded endogenous RT is generally expressed at higher levels in those cells that are characterized by a low differentiation states and high proliferation levels (e.g., transformed cells; reviewed by Sinibaldi-Vallebona et al., 2011), while differentiated, quiescent cells offer less permissive contexts for RT expression (Shi et al., 2007). In some embodiments, an L1 ORF2p of the presently disclosed subject matter has an amino acid sequence as set forth in SEQ ID NO: 57.

As disclosed herein, L1 ORF2p has been implicated in the reverse transcription of Alu elements, certain reverse transcription products of which accumulate in mammalian cells such as but not limited to cells of the eye. Alu elements are short, interspersed elements (SINEs) about 300 nucleotides in length, which amplify in primate genomes through a process of retroposition. Alu elements represent a significant fraction of noncoding DNA, particularly in humans. As disclosed herein, L1 ORFp reverse transcribes Alu nucleic acids and, in some embodiments, these reverse transcribed Alu nucleic acids (referred to herein as “Alu cDNAs”) accumulate in the cytoplasm of cells. In some embodiments, the Alu cDNAs are single stranded, and their presence correlates with progression of age-related macular degeneration (AGE), degeneration of retinal pigmented epithelium (RPE) cells, and geographic atrophy (GA) of the eye.

Thus, in some embodiments, methods for treating and/or preventing accumulation of Alu cDNAs in the cells of the eye are provided, wherein the method broadly comprise inhibiting the reverse transcription of Alu nucleic acids using RTase inhibitors generally, and L1 ORF2 inhibitors in particular. Various RTase inhibitors are known, including but not limited to L1 ORF2 inhibitors, nucleoside reverse transcriptase inhibitors (NRTIs), alkylated derivatives of NRTIs, and non-nucleoside reverse transcriptase inhibitors (NNRTIs). “RTase inhibitors”, in particular “L1 ORF2 inhibitors”, are known, and any RTase inhibitor or L1 ORF2 inhibitor can be employed in the methods of the presently disclosed subject matter. See e.g., Banuelos-Sanchez et al., 2019; U.S. Patent Application Publication No. 2009/0099060; U.S. Pat. Nos. 10,214,591; 10,294,220; and 10,371,703; each of which is incorporated by reference in its entirety.

Thus, contemplated within the scope of the phrase “RTase inhibitor”, in particular “L1 ORF2 inhibitor”, are in some embodiments inhibitory nucleic acids that target L1 ORF2 transcription products and antibodies that are specific for L1 ORF2p that bind to the L1 ORF2 to prevent its RTase activity. As used herein, the phrase “inhibitor nucleic acid” refers to a single stranded or double-stranded RNA or DNA, specifically RNA, such as triplex oligonucleotides, ribozymes, aptamers, small interfering RNA including siRNA (short interfering RNA) and shRNA (short hairpin RNA), antisense RNA, or a portion thereof, or an analog or mimetic thereof, that is capable of reducing or inhibiting the expression of a target gene or sequence. Inhibitory nucleic acids can act by, for example, mediating the degradation or inhibiting the translation of mRNAs which are complementary to the interfering RNA sequence. An inhibitory nucleic acid, when administered to a mammalian cell, results in a decrease (e.g., by 5%, 10%, 25%, 50%, 75%, or even 90-100%) in the expression (e.g., transcription or translation) of a target sequence. Typically, a nucleic acid inhibitor comprises or corresponds to at least a portion of a target nucleic acid molecule, or an ortholog thereof, or comprises at least a portion of the complementary strand of a target nucleic acid molecule. Inhibitory nucleic acids may have substantial or complete identity to the target gene or sequence, or may include a region of mismatch (i.e., a mismatch motif). The sequence of the inhibitory nucleic acid can correspond to the full-length target gene, or a subsequence thereof. In one aspect, the inhibitory nucleic acid molecules are chemically synthesized.

The specific sequence utilized in design of the inhibitory nucleic acids is a contiguous sequence of nucleotides contained within the expressed gene message of the target. Factors that govern a target site for the inhibitory nucleic acid sequence include the length of the nucleic acid, binding affinity, and accessibility of the target sequence. Sequences may be screened in vitro for potency of their inhibitory activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the content of which is incorporated herein by reference.

Phosphorothioate antisense oligonucleotides may be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. A peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Short interfering (si) RNA technology (also known as RNAi) generally involves degradation of an mRNA of a particular sequence induced by double-stranded RNA (dsRNA) that is homologous to that sequence, thereby “interfering” with expression of the corresponding gene. A selected gene may be repressed by introducing a dsRNA which corresponds to all or a substantial part of the mRNA for that gene. Without being held to theory, it is believed that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Exemplary siRNAs have sense and antisense strands of about 21 nucleotides that form approximately 19 nucleotides of double stranded RNA with overhangs of two nucleotides at each 3′ end.

siRNA has proven to be an effective means of decreasing gene expression in a variety of cell types. siRNA typically decreases expression of a gene to lower levels than that achieved using antisense techniques, and frequently eliminates expression entirely. In mammalian cells, siRNAs are effective at concentrations that are several orders of magnitude below the concentrations typically used in antisense experiments.

The double stranded oligonucleotides used to effect RNAi are specifically less than 30 base pairs in length, for example, about 29, 28, 27, 26, 25, 24, 23, 22, 21, 20, 19, 18, or 17 base pairs or less in length, and contain a segment sufficiently complementary to the target mRNA to allow hybridization to the target mRNA. Optionally, the dsRNA oligonucleotide includes 3′ overhang ends. Exemplary 2-nucleotide 3′ overhangs are composed of ribonucleotide residues of any type and may be composed of 2′-deoxythymidine residues, which lowers the cost of RNA synthesis and may enhance nuclease resistance of siRNAs in the cell culture medium and within transfected cells. Exemplary dsRNAs are synthesized chemically or produced in vitro or in vivo using appropriate expression vectors. Longer RNAs may be transcribed from promoters, such as T7 RNA polymerase promoters, known in the art.

Longer dsRNAs of 50, 75, 100, or even 500 base pairs or more also may be utilized in certain embodiments. Exemplary concentrations of dsRNAs for effecting RNAi are about 0.05 nM, 0.1 nM, 0.5 nM, 1.0 nM, 1.5 nM, 25 nM, or 100 nM, although other concentrations may be utilized depending upon the nature of the cells treated, the gene target and other factors readily identifies by one of ordinary skill in the art.

Compared to siRNA, shRNA offers advantages in silencing longevity and delivery options. Vectors that produce shRNAs, which are processed intracellularly into short duplex RNAs having siRNA-like properties provide a renewable source of a gene-silencing reagent that can mediate persistent gene silencing after stable integration of the vector into the host-cell genome. Furthermore, the core silencing ‘hairpin’ cassette can be readily inserted into retroviral, lentiviral, or adenoviral vectors, facilitating delivery of shRNAs into a broad range of cell types.

A hairpin can be organized in either a left-handed hairpin (i.e., 5′-antisense-loop-sense-3′) or a right-handed hairpin (i.e., 5′-sense-loop-antisense-3′). The shRNA may also contain overhangs at either the 5′ or 3′ end of either the sense strand or the antisense strand, depending upon the organization of the hairpin. If there are any overhangs, they are specifically on the 3′ end of the hairpin and include 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Specifically, there is only one phosphate group on the 5′ end of the region that will form the antisense strand following Dicer processing. In one exemplary embodiment, a right-handed hairpin can include a 5′ end (i.e., the free 5′ end of the sense region) that does not have a 5′ phosphate group, or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′-OH functional group (e.g., the 5′-most nucleotide is a 5′-deoxy nucleotide). In cases where the hairpin is a left-handed hairpin, preferably the 5′ carbon position of the 5′-most nucleotide is phosphorylated.

Hairpins that have stem lengths longer than 26 base pairs can be processed by Dicer such that some portions are not part of the resulting siRNA that facilitates mRNA degradation. Accordingly the first region, which may include sense nucleotides, and the second region, which may include antisense nucleotides, may also contain a stretch of nucleotides that are complementary (or at least substantially complementary to each other), but are or are not the same as or complementary to the target mRNA. While the stem of the shRNA can include complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

Modified shRNAs can retain the modifications in the post-Dicer processed duplex. In exemplary embodiments, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin. Also, Dicer processing of hairpins with this configuration can retain the 5′ end of the sense strand intact, thus preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality. Examples of exemplary modification patterns are described in detail in U.S. Patent Publication No. 2005/0223427 and PCT International Patent Publication Nos. WO 2004/090105 and WO 2005/078094, the disclosure of each of which is incorporated by reference herein in its entirety.

shRNA may comprise sequences that were selected at random, or according to a rational design selection procedure. For example, rational design algorithms are described in PCT International Patent Publication No. WO 2004/045543 and U.S. Patent Application Publication No. 2005/0255487, the disclosure of each of which is incorporated herein by reference in it entirety. Additionally, it may be desirable to select sequences in whole or in part based on average internal stability profiles (“AISPs”) or regional internal stability profiles (“RISPs”) that may facilitate access or processing by cellular machinery.

Ribozymes are enzymatic RNA molecules capable of catalyzing specific cleavage of mRNA, thus preventing translation. The mechanism of ribozyme action involves sequence specific hybridization of the ribozyme molecule to complementary target RNA, followed by an endonucleolytic cleavage event. The ribozyme molecules specifically include (1) one or more sequences complementary to a target mRNA, and (2) the well-known catalytic sequence responsible for mRNA cleavage or a functionally equivalent sequence (see e.g., U.S. Pat. No. 5,093,246, which is incorporated herein by reference in its entirety).

While ribozymes that cleave mRNA at site-specific recognition sequences can be used to destroy target mRNAs, hammerhead ribozymes may alternatively be used. Hammerhead ribozymes cleave mRNAs at locations dictated by flanking regions that form complementary base pairs with the target mRNA. Specifically, the target mRNA has the following sequence of two bases: 5′-UG-3′. The construction and production of hammerhead ribozymes is well known in the art and is described more fully in U.S. Pat. No. 5,633,133, the contents of which are incorporated herein by reference.

Gene targeting ribozymes may contain a hybridizing region complementary to two regions of a target mRNA, each of which is at least 5, 6, 7, 8, 9, 10, 11, 12, 13, 14, 15, 16, 17, 18, 19, or 20 contiguous nucleotides (but which need not both be the same length).

Hammerhead ribozyme sequences can be embedded in a stable RNA such as a transfer RNA (tRNA) to increase cleavage efficiency in vivo. In particular, RNA polymerase III-mediated expression of tRNA fusion ribozymes is well known in the art. There are typically a number of potential hammerhead ribozyme cleavage sites within a given target cDNA sequence. Specifically, the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Ribozymes also include RNA endoribonucleases (“Cech-type ribozymes”) such as the one which occurs naturally in Tetrahymena thermophile, described in PCT International Patent Application Publication No. WO 1988/04300. The Cech-type ribozymes have an eight base pair active site which hybridizes to a target RNA sequence where after cleavage of the target RNA takes place. In one embodiment, Cech-type ribozymes target eight base-pair active site sequences that are present in a target gene or nucleic acid sequence.

Ribozymes can be composed of modified oligonucleotides (e.g., for improved stability, targeting, etc.) and can be chemically synthesized or produced through an expression vector. Because ribozymes, unlike antisense molecules, are catalytic, a lower intracellular concentration is required for efficiency. Additionally, in certain embodiments, a ribozyme may be designed by first identifying a sequence portion sufficient to cause effective knockdown by RNAi. Portions of the same sequence may then be incorporated into a ribozyme.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. Nucleic acid molecules to be used in triple helix formation for the inhibition of transcription are specifically single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Inhibitory nucleic acids can be administered directly or delivered to cells by transformation or transfection via a vector, including viral vectors or plasmids, into which has been placed DNA encoding the inhibitory oligonucleotide with the appropriate regulatory sequences, including a promoter, to result in expression of the inhibitory oligonucleotide in the desired cell. Known methods include standard transient transfection, stable transfection and delivery using viruses ranging from retroviruses to adenoviruses. Delivery of nucleic acid inhibitors by replicating or replication-deficient vectors is contemplated. Expression can also be driven by either constitutive or inducible promoter systems. In some embodiments, expression may be under the control of tissue or development-specific promoters.

In some embodiments, an RTase inhibitor of the presently disclosed subject matter is an NNRTI. Various NNRTIs are known (see e.g., U.S. Patent Application Publication No. 2010/0029591, incorporated by reference herein in its entirety). is selected from the group consisting of efavirenz (EFV; see e.g., U.S. Patent Application Publication No. 2003/0124186, incorporated by reference herein in its entirety) and delaviridine (DLV; see e.g., U.S. Pat. No. 9,421,204, incorporated by reference herein in its entirety).

II.A. Subjects

Thus, in some embodiments the presently disclosed subject matter provides a method for treating subjects comprising administering to the subjects a composition, wherein the composition comprises an RTase inhibitor, in some embodiments a cytoplasmic RTase inhibitor, and in some embodiments a L1 ORF2p.

As used herein, the phrase “treating an injury to a tissue or organ in a subject” refers to both intervention designed to ameliorate the symptoms of causes of the injury in a subject (e.g., after initiation of a disease process) as well as to interventions that are designed to prevent the injury from occurring in the subject. Stated another way, the terms “treating” and grammatical variants thereof are intended to be interpreted broadly to encompass meanings that refer to reducing the severity of and/or to curing a disease or disorder, as well as meanings that refer to prophylaxis. In this latter respect, “treating” refers to “preventing” or otherwise enhancing the ability of the subject to resist the effects of a disease process or injury.

IIB. Formulations

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

In some embodiments, an RTase inhibitor of the presently disclosed subject matter is provided as a cell-permeable, non-immunogenic cholesterol-conjugated siRNA. Methods for conjugating carbohydrates to oligonucleotides such as but not limited to siRNAs are disclosed in U.S. Patent Application Publication No. 2019/0184018, the entire disclosure of which is incorporated herein by reference.

II.C. Administration

Suitable methods for administration of the compositions of the presently disclosed subject matter include, but are not limited to intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration; or intravenous administration.

In some embodiments the presently disclosed compositions comprise a pharmaceutically acceptable carrier, which in some embodiments can be pharmaceutically acceptable for use in a human.

II.D. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly. After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods Employed in Examples 1-3

Animals. Wild-type (WT) C57BL/6J mice and Brown Norway BN/RijHsd rats were purchased from The Jackson Laboratory (Bar Harbor, Me., United States of America) and Envigo (Frederick, Md., United States of America), respectively. Casp1^{-/-} Casp4^{129mt/129mt} (Casp1/4 dko) and Nlrp3^{-/-} mice were obtained from G. Nunez (University of Michigan, Ann Arbor, Mich., United States of America), and Mb21d1 mice were obtained from K. A. Fitzgerald (University of Massachusetts Medical School, Worcester, Mass., United States of America). Rice rats (Oryzomys palustris) have been previously described (Casavant et al., 2000; Grahn et al., 2005; Yang et al., 2014). For all procedures, anaesthesia was achieved by intraperitoneal injection of 100 mg/kg ketamine hydrochloride (Fort Dodge Animal Health, Fort Dodge, Iowa, United States of America) and 10 mg/kg xylazine (Phoenix Scientific, Inc., San Marcos, Calif., United States of America), and pupils were dilated with topical 1% tropicamide and 2.5% phenylephrine hydrochloride (Alcon Laboratories, Fort Worth, Tex., United States of America). Mice and rats were treated in accordance with the guidelines of the University of Virginia (Charlottesville, Va., United States of America) and University of Kentucky (Lexington, Ky., United States of America) Institutional Animal Care and Use Committees and the Association for Research in Vision and Ophthalmology. Both male and female mice between 6-10 weeks of age were used, and male rats between 2-3 months of age were used.

Fundus photography. Fundus imaging of dilated mouse and rat eyes was performed using a TRC-50 IX camera (Topcon Medical Systems, Inc., Oakland, N.J., United States of America) linked to a digital imaging system (Sony Corporation of America, New York, N.Y., United States of America).

Human tissue. All studies on human tissue followed the guidelines of the Declaration of Helsinki. The study of deidentified tissue collected from deceased individuals and obtained from various eye banks in the United States was exempted from IRB review by the University of Virginia Institutional Review Board for Health Sciences Research, in accordance with US Health & Human Services human-subjects regulations. Donor eyes from patients with geographic atrophy (GA) or age-matched patients without age-related macular degeneration (AMD) were obtained from various eye banks. These diagnoses were confirmed through ophthalmic examination of dilated eyes before acquisition of the tissues or eyes or after examination of the eye globes post-mortem. Enucleated donor eyes isolated within six hours post-mortem were immediately preserved in RNAlater Solution (Thermo Fisher Scientific, Waltham, Mass., United States of America) or formalin. The neural retina and sclera were removed, and tissues comprising both macular RPE and choroidal tissue were snap frozen in liquid nitrogen. For in situ hybridization, eyes were transferred to 70% v/v ethanol after fixation. For eyes with GA, the RPE and choroidal tissues were collected and divided into atrophic, junctional, and peripheral areas4. Frozen section of eyes with Leber congenital amaurosis, Joubert syndrome, Stargardt macular dystrophy, or autosomal recessive retinitis pigmentosa have been previously described (Bonilha et al., 2011; Bonilha et al., 2015a; Bonilha et al., 2015b; Bonilha et al., 2016).

Chemicals. The NRTIs lamivudine (3TC) and stavudine (d4T) as well as the NNRTIs efavirenz (EFV), delavirdine (DLV), and nevirapine (NVP), and ATP were purchased from Sigma-Aldrich Corp. (St. Louis, Mo., United States of America). Lipopolysaccharide (LPS) was purchased from InvivoGen (San Diego, Calif., United States of America) and azidothymidine triphosphate (AZT-TP) from TriLink BioTechnologies (San Diego, Calif., United States of America). A trimethyl-modified version of 3TC (TM-3TC) and a diethyl-modified version of AZT (DE-AZT) were synthesized as previously described (U.S. Patent Application Publication No. 2018/0044327, incorporated by reference herein in its entirety). The nuclear-targeting cyclic peptides (Cpep) and Cpep-conjugated 3TC (Cpep-3TC), and cytoplasmic-targeting PA-4 3TC have been previously described (Mandal et al., 2011; Nasrolahi et al., 2013).

In vitro transcribed Alu RNA and Alu mutant RNA. Alu RNA was synthesized from a linearized plasmid containing a consensus Alu Y sequence with an adjacent 5′ T7 promoter (Tarallo et al., 2012), subjected to AMPLISCRIBE™ T7-FLASH™ Transcription kit (Epicentre, Madison, Wis., United States of America) according to the manufacturer's instructions. DNase-treated RNA was purified using MEGACLEAR™ (Ambion Inc. Austin, Tex., United States of America) and integrity confirmed by agarose gel electrophoresis. Alu RNA with G25C mutation, which lies within a predicted SRP9/14 binding site in the Alu RNA left arm, was synthesized from a linearized plasmid containing the G25C mutation as described above.

Assessment of RPE degeneration. Subretinal injections were performed as previously described (Kaneko et al., 2011; Tarallo et al., 2012; Kerur et al., 2013; Fowler et al., 2014; Kim et al., 2014). Seven days after subretinal injection, RPE health was assessed by fundus photography and immunofluorescence staining of zonula occludens-1 (ZO-1) on RPE flat mounts (whole mount of posterior eye cup containing RPE and choroid layers). Mouse RPE/choroid flat mounts were fixed with 2% paraformaldehyde, stained with rabbit polyclonal antibodies against mouse ZO-1 (1:100; Invitrogen Corp. Carlsbad, Calif., United States of America) and visualized with Alexa-594 (Invitrogen Corp.). All images were obtained by microscopy (Model SP-5 (Leica Microsystems, Inc., Buffalo Grove, Ill., United States of America) or Nikon MR confocal microscope system (Nikon Instruments Inc. Melville, N.Y., United States of America). Imaging was performed by an operator blinded to the group assignments.

Quantification of RPE degeneration. Quantification of RPE degeneration was performed using two methodologies (binary assignment and cellular morphometry) as described previously (Kerur et al., 2018): Binary assignment (healthy versus unhealthy; Kerur et al., 2013; Fowler et al., 2014; Kim et al., 2014; Kerur et al., 2018) was independently performed by two blinded raters (inter-rater agreement=99.7%; Pearson r2=0.986, P<0.0001; Fleiss x=0.993, P<0.0001). Quantifying cellular morphometry for hexagonally packed cells was performed in semi-automated fashion by three masked graders by adapting our previous analysis of the planar architecture of corneal endothelial cell density (Ach et al., 2015), which resembles the RPE in its polygonal tessellation. Polymegethism (coefficient of variation of cell size), a prominent geometric feature of RPE cells in GA (Kaneko et al., 2011; Dridi et al., 2012; Grossniklaus et al., 2013; Ach et al., 2015), was quantified using the Konan Cell Check software (Ver. 4.0.1), a commercial U.S. FDA-cleared software that has been used for registration clinical trials, as previously described (Kerur et al., 2018).

Subretinal and intravitreous injections. Subretinal injections (1 μl for mice, 3 μl for rat) or intravitreous injections (0.5 μl for mice, 2 μl for rat) in mice or rat were performed using a 35-gauge needle (Ito Co. Fuji, Japan). In vivo transfection of Alu RNA or mutant Alu RNA (3.6-300 ng per eye); Alu complementary DNA (cDNA; 0.0036-90 ng per eye); Alu reverse sequence cDNA (3.6-90 ng per eye); or 7SL cDNA (3.6-90 ng per eye) was performed using 10% NEUROPORTER™ transfection reagent (Genlantis, San Diego, Calif., United States of America) as previously described (Kaneko et al., 2011; Tarallo et al., 2012). 3TC, TM-3TC, Cpep-3TC, Cpep, PA-4 3TC, EFV, DLV, or NVP (50 μM/1 μl or 500 μM/1 μl) were administered by intravitreous injection (Mandal et al., 2011; Nasrolahi et al., 2013). 1 μl of cholesterol-conjugated siRNAs (2 μg/μl) targeting mouse LINE1 or Luc (luciferase control; Dharmacon, Lafayette, Colo., United States of America) were subretinally injected three days prior to Alu RNA or vehicle administration. Similarly, in Oryzomys palustris, rat L1 plasmids expressing ORF1p, ORF2p, reverse transcriptase-deficient ORF2p (pORF2 (RT-)), or endonuclease deficient ORF2p (pORF2 (EN-)) were delivered via subretinal injection three days prior to administering Alu RNA or vehicle. The choice of eye for experimental versus control injection was chosen randomly. Rat L1 plasmids expressing ORF1 and ORF2 have been described previously (Kirilyuk et al., 2008). The ORF2 (EN-) construct contained mutations D207A and H232A, which, by CLUSTALW alignment, correspond to human D205A and H230A (see Feng et al., 1996) in the endonuclease domain of ORF2p. The ORF2 (RT-) construct contained mutation D703A, which, by CLUSTALW alignment, corresponds to human D702A (see Moran et al., 1996) in the reverse transcriptase domain of ORF2p.

cDNA synthesis. Single-stranded Alu cDNA, Alu reverse sequence cDNA, and 7SL cDNA were isolated from biotinylated double-stranded PCR products synthesized from a linearized plasmid containing a consensus Alu Y, Jb, Sxl, Sx, Sz, or 7SL sequence using DYNABEADS® M-270 Streptavidin (Life Technologies, Inc., Carlsbad, Calif., United States of America), then purified using Qiaquick PCR purification kit (Catalogue No. #28104, QIAGEN, Germantown, Md., United States of America; Wakimoto et al., 2014). Briefly, PCR products were biotinylated on one strand by synthesis with a biotinylated primer (forward 5′-biotin-GGGCCGGGCGCGGTG-3′ (SEQ ID NO: 1) and reverse 5′-GTACCTTTAAAGAGACAGAGTCTCGC-3′ (SEQ ID NO: 2) for Alu Y, forward 5′-biotin-GCCTGTAATCCCAGCACTTT-3′ (SEQ ID NO: 3) and reverse 5′-GAGACGGAGTCTCGCTCTG-3′ (SEQ ID NO: 4) for Alu Sx, Sxl and Sz, forward 5′-biotin-GCCTGTAATCCCAGCACTTT-3′ (SEQ ID NO: 3) and reverse 5′-CGGAGTCTCGCTCTGTCG-3′ (SEQ ID NO: 5) for Alu Jb, forward 5′-biotin-CGTGCCTGTAGTCCCAGCTA-3′ (SEQ ID NO: 6) and reverse 5′-AGACGGGGTCTCGCTATGTT-3′ (SEQ ID NO: 7) for 7SL, forward 5′-GGGCCGGGCGCGGTG-3′ (SEQ ID NO: 1) and reverse 5′-biotin-GTACCTTTAAAGAGACAGAGTCTCGC-3′ (SEQ ID NO: 2) for reverse sequence Alu). DYNABEADS® M-270 Streptavidin magnetic beads were used to capture the biotin-tagged PCR product. The PCR product was heated at 95° C. for 10 minutes for strand separation, and isolation of the non-biotinylated strand was performed using a magnetic stand followed by alcohol precipitation according to the manufacturer's instructions.

Western blotting. Cells and tissue were homogenized in RIPA buffer (Sigma-Aldrich) with protease and phosphatase inhibitors (Roche) or lysed directly in Laemmli buffer. Protein concentration was determined using Pierce BCA Protein Assay Kit (Thermo Fisher Scientific). Equal quantities of protein (10-50 μg) prepared in Laemmli buffer were resolved by SDS-PAGE on NOVEX® Tris-Glycine Gels (Invitrogen), or MINI-PROTEAN® TGX™ Precast Protein Gels (Bio-Rad Laboratories, Inc., Hercules, Calif., United States of America) and transferred onto Immobilon-FL PVDF membranes (0.2 or 0.45 μm; MilliporeSigma, Burlington, Mass., United States of America). The transferred membranes were blocked in ODYSSEY® Blocking Buffer (PBS) for 1 hour at room temperature and then incubated with primary antibody at 4° C. overnight. Immunoreactive bands were visualized using species-specific secondary antibodies conjugated with IRDYE®. The blot images were captured on ODYSSEY® imaging systems. Rabbit polyclonal anti-human and mouse caspase-1 antibodies (1:500; Catalogue #3019-100, Biovision Inc., Milpitas, Calif., United States of America; 1:1,000, Catalogue #AHZ0082, Invitrogen; 1:200, Catalogue #sc-514, Santa Cruz Biotechnology, Santa Cruz, Calif., United States of America; 1:1,000; Catalogue #ab108362, Abcam, Cambridge, MNassachusetts, United States of America), mouse monoclonal anti-mouse caspase-1 (1:1,000; Catalogue #AG-20B-0042-C100, AdipoGen Corp., San Diego, Calif., United States of America), mouse monoclonal anti-human TBP (1:1000; Catalogue #ab51841, Abcam), rabbit polyclonal anti-mouse LINE-1 (1:200; Catalogue #sc-67198, Santa Cruz Biotechnology), mouse monoclonal anti-human L1 ORF1p (1:1,000, gift of K. H. Burns, Johns Hopkins University School of Medicine, Baltimore, Md., United States of America), mouse monoclonal anti-human L1 ORF2p (1:100; see De Luca et al., 2016), rabbit polyclonal anti-human vinculin (1:2,000, Sigma-Aldrich Cat #V4139), mouse monoclonal anti-β-actin (1:50,000; Catalogue #A2228, Sigma-Aldrich), or mouse monoclonal anti-chicken tubulin (1:5,000; Catalogue #T6199, Sigma-Aldrich) were used.

Cell culture and transfection. Primary mouse and human RPE cells were isolated as previously described (Kerur et al., 2018). All cells were maintained at 37° C. in a 5% CO2 environment. Mouse RPE cells were cultured in DMEM supplemented with 20% FBS and penicillin/streptomycin antibiotics at standard concentrations; primary human RPE cells were maintained in DMEM supplemented with 10% FBS and antibiotics. The human RPE cell line ARPE19 was cultured as previously described (Kerur et al., 2018) and maintained in DMEM-F12 containing penicillin/streptomycin, Fungizone, and gentamicin. HEK293T cells were cultured in DMEM with 10% fetal bovine serum (FBS) with 100 U/ml penicillin/streptomycin and 2 mM L-glutamine. Primary wild-type mouse bone marrow-derived macrophages (BMDMs) were isolated, and cultured in Iscove's Modified Dulbecco's Medium (IMDM) supplemented with 30% L929 supernatant containing macrophage-stimulating factor, nonessential amino acids, sodium pyruvate, 10% FBS and antibiotics, 50 μM β-mercaptoethanol (Seo et al., 2015), and serum starved in IMDM with 1% FBS and 100 U/ml penicillin/streptomycin overnight prior to LPS stimulation. NRTIs or NNRTIs were added 30 minutes prior to LPS stimulation and again 30 minutes prior to ATP activation. LPS (100 ng/mL) was added for 3-4 hours prior to the addition of ATP. Cell lysates were collected 30 minutes after addition of ATP (5 mM). Primary human subcutaneous pre-adipocytes (Catalogue No. PCS-210-010, ATCC, Manassas, Va., United States of America) and primary human dermal fibroblasts (Catalogue No. PCS-201-012, ATCC) were grown in fibroblast basal medium with fibroblast growth kit for low serum (ATCC).

Umbilical artery vascular smooth muscle cells (Catalogue No. CC-2579, Lonza, Morristown, N.J., United States of America) were grown in SMGM™-2 Smooth Muscle Growth Medium-2 BULLETKIT™ (Lonza). Primary human skeletal myoblasts (Catalogue No. A11440, Thermo Fisher Scientific) were grown in DMEM with 2% horse serum. Primary human epidermal keratinocytes (Catalogue NO. CO215C, Thermo Fisher Scientific) were grown in EPILIFE® Medium (Thermo Fisher Scientific). Human umbilical vein endothelial cells (HUVEC) were grown in HUVEC EGM™-2 Media (Lonza). Primary human peripheral blood mononuclear cells (Catalogue NO. PCS-800-011, ATCC) were directly used without culture for experiments. RPMI 1640 medium with 10% human serum with human GM-CSF (Miltenyi Biotec Inc., Auburn, Calif., United States of America) was used as media during the experiment. Transfections were performed according to the manufacturer's instructions (LIPOFECTAMINE® 2000, Invitrogen). For transfection of primary human peripheral blood mononuclear cells, HiPerFect Transfection Reagent (Qiagen) was used as previously described (Troegeler et al., 2014). NRTIs were administered 60 minutes before transfection and added again upon replacement of media at 8 hours. To induce acid injury or osmotic stress, primary human RPE cells were treated with HCl (pH 4.0 media) or H₂O for 30 minutes, 1 hour, and 2 hours at 37° C. in 5% CO2, respectively. NIH3T3 Tet-ON cells were cultured in DMEM with 10% tetracycline-free fetal bovine serum (FBS) with 100 U/ml penicillin/streptomycin. NIH3T3 Tet-ON cells were transfected with pLD401 (see Taylor et al., 2013; gift of J. D. Boeke, Institute for Systems Genetics, NYU Langone Health, New York, New York, United States of America) and human L1 ORF1p abundance was monitored after 24 hours of doxycycline induction.

Upregulation of Alu RNA levels in vitro. In vitro transcribed Alu RNA, DICER1 antisense (AS) oligonucleotide (5′-GCUGACCTTTTTGCTUCUCA-3; SEQ ID NO: 8), or control scrambled AS (5′-TTGGTACGCATACGTGTTGACTGTGA-3; SEQ ID NO: 9; Integrated DNA Technologies, Redwood City, Calif., United States of America) were transfected into human and mouse RPE cells using LIPOFECTAMINE® 2000 (Invitrogen) according to the manufacturer's instructions. Heat stress was induced by placing cells in a 42° C. or 45° C. incubator for 20 minutes and then allowed to recover at 37° C. for 1 hour (Liu et al., 1995).

Sequencing. RPE cells were lysed with gentle extraction buffer prepared in 1×PBS containing 1% v/v Triton X-100 (Sigma-Aldrich) and 1 mM EDTA for 15 minutes on ice. Lysate was centrifuged at 1000×g for 10 minutes at 4° C. to pellet-out nuclei. The lysate supernatant was used as the cytoplasmic fraction. Cytoplasmic samples were size-fractionated on a Blue Pippin device (Sage Science, Inc., Beverly, Mass., United States of America) to exclude large molecular weight DNA >1500-nt. Blue Pippin samples were enriched for ssDNA as determined by Qubit for ssDNA and dsDNA pre- and post-fractionation. Pippin-fractionated ssDNA samples were converted to dsDNA by the Seq Plex Enhanced DNA Amplification Kit (Sigma-Aldrich, SEQXE) without additional fragmentation to enrich for DNA between 200-800 bp. Amplification was monitored by RT-PCR, and cycle number (20-25 cycles) was set as 2-3 cycles after the amplification plateau, as suggested by the manufacturer. 1 μg of dsDNA library was prepared for sequencing with the NEXTFLEX® Rapid DNA Sequencing Kit (Bioo Scientific, Austin, Tex., United States of America). Samples were sequenced on the HiSeq 2500 SE50 platform (Illumina, Inc., Madison, Wis., United States of America. The quality of reads was assessed with FastQC (Babraham Bioinformatics Group, Babraham Institute, Babraham, Cambridge, United Kingdom). The reads were then mapped to human reference chromosomes (hg19: chrl-22, X, Y, M) by two methods: MapSplice30 and STAR31. Both of these methods were configured to report the best alignment of each read with minimal mismatches. Only uniquely aligned reads were retained for further analysis. The GTF file containing the genomic locations of all Alu species and their family classifications was obtained from the UCSC Genome Browser on the World Wide Web (University of California Santa Cruz Genomics Institute, Santa Cruz, Calif., United States of America)). Taking the read alignment and the GTF file as input, FeatureCounts (Liao et al., 2014) was used to calculate the total read count in each Alu subfamily.

In situ hybridization. Cells or RPE flat mounts were fixed in 4% PFA/PBS for 20 minutes. For Alu cDNA detection, all samples were treated with RNase A. To confirm whether the target was single-stranded DNA, 51 nuclease (Thermo Fisher Scientific) was treated for 30 minutes at room temperature. RNA probes, prepared from linearized Alu cDNA templates using a T7 fluorescein RNA labeling kit or T7 DIG RNA labeling kit (Roche), were hybridized overnight at 37° C. in a mixture containing 10% dextran sulphate, 2 mM vanadyl-ribonucleoside complex, 0.02% RNase-free BSA, 40 μg E. coli tRNA, 2×SSC, 50% formamide, and RNA probe. Cells were then subjected twice to stringent washing at 50° C. in 50% formamide, 0.1×SSC for 30 minutes. Following washing, samples were incubated with a horseradish peroxidase (HRP)-conjugated anti-fluorescent antibody (Catalogue #NEF710001EA, PerkinElmer, Waltham, Mass., United States of America) or HRP-conjugated anti-DIG antibody (Catalogue # NEF832001EA, PerkinElmer) at a 1:200 dilution. Visualization of fluorescein-labelled probe was performed with the TSA™ plus fluorescence system or the TSA™ plus Cy5 system (PerkinElmer). The fluorescent or Cy5 signals were detected using a Leica SP-5 or MR Nikon confocal microscope system.

Equator blotting. An “equator blot” is a combination of classic “Southern” and “northern” blot procedures. An equator blot is similar to a Southern blot in that it probes for target DNA sequence, yet unlike a typical Southern blot, it does not involve restriction enzyme digest of the DNA. Instead, the DNA is run without enzyme digestion prior to hybridization, per the typical northern blot procedure. Hence, the procedure of hybridization of undigested DNA is referred to herein as an equator blot. Total nucleic acid or nuclear and cytoplasmic fractions were extracted from cells as described below. For human tissue, DNA and RNA were extracted using DNA and RNA Purification Kit (Epicentre); RNase A was added for DNA isolation, and DNase I was added for RNA isolation. DNA/RNA samples were run on 10% TBE-urea gels (BioRad) according to the manufacturer's instructions. Samples were transferred and UV crosslinked to a HyBond N+ nylon membrane and blotted for Alu RNA, Alu cDNA, and U6 RNA. U6 biotinylated oligonucleotide probe was synthesized by Integrated DNA Technologies (5′-CACGAATTTGCGTGTCATCCTT-biotin-3′; SEQ ID NO: 10). Alu RNA/Alu cDNA biotinylated oligonucleotide probe was synthesized by PCR from a linearized plasmid containing a consensus Alu Y element as above using the following primers: for Alu cDNA detection (forward 5′-biotin-GGGCCGGGCGCGGTG-3′; SEQ ID NO: 1 and 5′-GTACCTTTAAAGAGACAGAGTCTCGC-3′; SEQ ID NO: 2), for Alu RNA detection (forward 5′-GGGCCGGGCGCGGTG-3′; SEQ ID NO: 1 and 5′-biotin-GTACCTTTAAAGAGACAGAGTCTCGC-3; SEQ ID NO: 2) and then purified. Blots were developed with the Pierce chemiluminescent nucleic acid detection kit (Thermo Fisher Scientific). The blot images were captured on ODYSSEY® imaging systems.

Nuclear and cytoplasmic fractionation. Briefly, cells were collected and lysed with gentle extraction buffer prepared in 1×PBS containing 1% v/v Triton X-100 (Sigma-Aldrich) and 1 mM EDTA for 15 minutes on ice. Lysates were vortexed and centrifuged at 1,000×g for 10 minutes at 4° C. For cytoplasmic fractionation, the supernatant was collected, subjected to repeated centrifugation four times, and then purified using a DNA purification column (Enzymax LLC, Lexington, Ky., United States of America). Lysis buffer was added to the pellet for reconstitution. Lysate supernatant was vortexed and further centrifuged at 13,000×g for 2 minutes at room temperature. Lysate supernatant was used as the nuclear fraction and purified using a DNA purification column (Enzymax). For cDNA detection, samples were treated with RNase A (Ambion, Inc.) for 30 minutes at 37° C. To confirm nuclear and cytoplasmic fractionation, cytoplasmic and nuclear RNA were isolated from primary human RPE cells and run on a 0.9% agarose gel to assess genomic DNA, 18S rRNA, and 28S rRNA. Levels of cytoplasmic and nuclear U6 RNA and tRNA were also measured by PCR. PCR reactions were performed using the following primers: U6 (forward 5′-GTGCTCGCTTCGGCAGCACATATAC-3′ (SEQ ID NO: 11); reverse 5′-AAAAATATGGAACGCTTCACGAATTTG-3′; SEQ ID NO: 12); tRNA (forward 5′-AGCAGAGTGGCGCAGCGG-3′ (SEQ ID NO: 13); reverse 5′-GATCCATCGACCTCTGGGTTA-3′; SEQ ID NO: 14). A primer set within an intron of GPR15 was used to measure genomic DNA was as previously described (Hoebeeck et al., 2005; D'Haene et al., 2010). Cytoplasmic and nuclear levels of GPR15 were directly amplified by real-time PCR (without RT) using the following primers: GPR15 (forward 5′-GGTCCCTGGTGGCCTTAATT-3′ (SEQ ID NO: 15); reverse 5′-TTGCTGGTAATGGGCACACA-3′; SEQ ID NO: 16).

Alu cDNA detection by real-time PCR. Cells were collected after counting the cell number and the cytoplasmic fraction was treated with RNase A (Ambion). The RNase-treated cytoplasmic fraction was purified with PCR clean-up kit (QIAquick, Qiagen). Then samples were directly amplified by real-time quantitative PCR (7900 HT Fast Real-Time PCR system, Applied Biosystems, Foster City, Calif., United States of America) with Power SYBR Green Master Mix. Primers were specific for human Alu cDNA (forward 5′-TTAGCCGGGAGTGGTGTCGG-3′ (SEQ ID NO: 17); and reverse 5′-ACCTCCCGGGTTCACGCCATT-3′; SEQ ID NO: 18). The copy number of Alu cDNA was calculated using standard curves that were obtained using serial dilutions of the plasmids containing an Alu Y sequence. Alu cDNA copy number was normalized to cell number.

A method to purify and amplify the reverse transcribed single-stranded DNA and minimize the amplification from contaminating genomic DNA (Alu c-PCR) was developed. First, total cell lysate was fractionated to yield nuclear and cytoplasmic fractions as above. The purified cytoplasmic fraction was poly-A-tailed with TdT (New England Biolabs, Ipswich, Mass., United States of America) for 30 minutes at 37° C. according to the manufacturer's instructions. The poly-A-tailed template was annealed and extended by a PolyT-anchored adapter primer (TAV oligo). These anchored DNAs were amplified using anchor-specific primer and reverse primer specific for Alu. The TAV oligo contains a unique 22-nt anchor sequence at the 5′-end followed by 18 thymidines (dT), and ends with a V nucleotide (where V represent adenosine (A), guanosine (G), or cytidine (C)). The TAV oligonucleotide has the sequence 5′-GACCACGCGTATCGATGTCGACTTTTTTTTTTTTTTTTTTV-3 (SEQ ID NO: 19), the anchor primer has the sequence 5′-GACCACGCGTATCGATGTCGAC-3′ (SEQ ID NO: 20; which corresponds to nucleotides 1-22 of the TAV oligonucleotide of SEQ ID NO: 19), and Alu specific primer has the sequence 5′-ACCTCCCGGGTTCACGCCATT-3′ (SEQ ID NO: 21). The Alu c-PCR method specifically detects linear Alu cDNA while avoiding detecting the circular form of extrachromosomal Alu DNAs. In this method, the first step is poly-A-tailing of linear Alu cDNAs; this poly A-tailed DNA primes the synthesis of DNA by poly T-anchored adapter primer. These anchored DNAs are then amplified by using a primer specific for the adapter and another primer specific for Alu. Circular Alu dsDNAs may already have a poly A region that can prime the synthesis of DNA by the poly T-anchored primer; however, this anchored DNA cannot be amplified by using the primer specific for Alu.

Real-time PCR. For human tissue, total RNA was extracted using MASTERPURE™ Complete DNA and RNA Purification Kit (Epicentre) according to the manufacturer's recommendation. The RT products (cDNA) were amplified by real-time quantitative PCR (Applied Biosystems 7900 HT Fast Real-Time PCR system) with Power SYBR Green Master Mix. Relative gene expression was determined by 2^{-ΔΔCt} method using 18S rRNA as an internal control. Primers for real-time PCR were, for human L1 ORF1 (forward 5′-AGGAACAGCTCCGGTCTACA-3; (SEQ ID NO: 21) and reverse 5′-GATGAACCCGGTACCTCAGA-3′; SEQ ID NO: 22), for human L1 ORF2 (forward 5′-ACTGGCCATCAGAGAAATGC-3′ (SEQ ID NO: 23) and reverse 5′-CAGCACCTGTTGTTTCCTGA-3′; SEQ ID NO: 24), for human 18S rRNA (forward 5′-CGCAGCTAGGAATAATGGAATAGG-3′ (SEQ ID NO: 25) and reverse 5′-GCCTCAGTTCCGAAAACCAA-3′; SEQ ID NO: 26), for rat L1 ORF1 (forward 5′-GCCAGAAGATCCTGGACTGAT-3′ (SEQ ID NO: 27); reverse 5′-GTAACCTGGGCTGGCATTTG-3′; SEQ ID NO: 28), for rat L1 ORF2 (forward 5′-GCAGATCGATCCATGCTTATCAC-3′ (SEQ ID NO: 29) and reverse 5′-GATGTGGAGGTCCTTGATCCA-3′; SEQ ID NO: 30), for hZNF66 (forward 5′-GCTCCTCTAACCTTACTAAACAC-3′ (SEQ ID NO: 31) and reverse 5′-TTTGCCACATTTATTGCACT-3′; SEQ ID NO: 32), for hZFP30 (forward 5′-ATAGAAGCCTTTCATCACCT-3′ (SEQ ID NO: 33) and reverse 5′-TTGCCCTGAAATACAGTTCC-3′; SEQ ID NO: 34), for hGBA2 (forward 5′-CCCAAAAGAGACGGACTGCT-3′ (SEQ ID NO: 35) and reverse 5′-AGCCCATGCCTATATGCTT-3′; SEQ ID NO: 36), for hLINC01873 (forward 5′-ACGGGAGGACATTCAAACCAA-3′ (SEQ ID NO: 37) and reverse 5′-ATCTTCCATCGCTGATACCCT-3′; SEQ ID NO: 38), for mZfp933 (forward 5′-ACAGCATAGTAATCTCCGAA-3′ (SEQ ID NO: 39) and reverse 5′-AAGATGATAGTAACGTGCAA-3′; SEQ ID NO: 40), for mHfe2 (forward 5′-GCCAACGCTACCACCATCCG-3′ (SEQ ID NO: 41) and reverse 5′-ACGTGACTCCCAAGGTTAGCA-3′; SEQ ID NO: 42), for mZfp945 (forward 5′-GGCTCATATCTTAGAATGCAC-3′ (SEQ ID NO: 43) and reverse 5′-GATCTGTCGCAATTACCAC-3′; SEQ ID NO: 44), and for mPias4 (forward 5′-AGCTTCCGAGTATCAGACCT-3′ (SEQ ID NO: 45) and reverse 5′-TGCACTCTTCTTGGCATAGCG-3; SEQ ID NO: 46).

In vitro reverse transcriptase (RT) activity. In vitro reverse transcriptase (RT) activity in nuclear and cytoplasmic protein fractions was assessed using an Alu RNA-templated reaction. The RT reaction was carried out in a 20 μl reaction mix containing Alu RNA template (10 ng); Alu primer (10 μmol); dNTPs mix; cytoplasmic or nuclear protein, and Quantiscript RT Buffer (Qiagen). The reaction mixture was incubated at 42° C. for 30 minutes. The resulting cDNA was quantified by qPCR using Alu RNA template-specific primers. The reaction to evaluate self-priming activity of Alu RNA was carried out in the absence of priming oligos in a 20 μl reaction mix containing: Alu RNA with 3′-U tail; dNTP mix; cytoplasmic protein from mouse RPE cells; and Quantiscript RT Buffer (Qiagen) as described above. The resulting cDNA product was quantified by qPCR using Alu RNA template-specific primers. Alu RNA tailed on the 3′ end with chain terminator dideoxy thymidine base (ddTTP) was generated using TdT (New England Biolabs (NEB)) according to the manufacturer's instructions. Alu RNA tailed on the 3′ end with chain terminator cordycepin tri-phosphate was generated using PAP (NEB) according to the manufacturer's instructions.

Alu retrotransposition reporter assay. Retrotransposition reporter assays were carried out as follows. Briefly, 2×10⁵ HeLa cells were plated in 6-well tissue culture dish and, one day later, were transfected in triplicate using FUGENE® 6 (Promega Corporation, Madison, Wis., United States of America) with 1 μg of the wild type L1 reporter plasmid pJM101/L1.3Δneo as described previously (Wei et al., 2001), pORF2, pORF2 (RT-), or pORF2 (EN-), along with 1 μg of Alu retrotransposition indicator construct Alu neo (gift of John V. Moran, University of Michigan Medical School, Ann Arbor, Mich., United States of America) and Alu RNA with G25C mutation. After 72 hours, cells were provided DMEM containing 600 μg/mL G418 and 100 μg/mL penicillin/streptomycin (Cellgro, Manassas, Va., United States of America). Fourteen days later, the plates were washed with methanol, Giemsa stained, and photographed. Colonies were counted manually using ImageJ (see the website of the U.S., National Institutes of Health). A total of 1 μg of Alu-neo with 1 μg empty driver vector was used as a negative control.

L1-EGFP retrotransposition reporter assay. The enhanced green fluorescent protein (EGFP) cell culture L1 retrotransposition assay was performed as previously described (Ostertag et al., 2000) in HeLa cells. Cells were transfected with a plasmid expressing a function human L1 element tagged with an EGFP reporter (RPS-EGFP) (gift of H. H. Kazazian, Johns Hopkins School of Medicine, Baltimore, Md., United States of America) in the presence of vehicle, 3TC, or TM-3TC (50 μM). Transfected cells were selected in puromycin-containing medium. Seven days after transfection, cells that underwent retrotransposition (EGFP-positive) were assayed by flow cytometry. Cells were gated based on background fluorescence of control plasmid JM111-EGFP (gift of H. H. Kazazian), which has two point mutations in L1 ORF1 abolishing retrotransposition capability.

Lentivirus transduction assay. HeLa cells were plated in 96-well plates in the presence or absence of a GFP-expressing lentivirus (MOI 10) (Genetic Technology Core; COBRE, University of Kentucky, Lexington. Ky., United States of America), with or without 3TC (50 μM) or TM-3TC (50 μM). Cells were incubated for 48 hours, stained with Hoechst, and then imaged on a BIOTEK® plate reader (BioTek Instruments, Inc. Winooski, Vt., United States of America). Representative images were captured, and the numbers of GFP⁺ and Hoechst⁺ cells per field of view (FOV) were automatically counted.

Immunofluorescence staining. For Alu RNA and L1 ORF2p co-localization, after 48 hours of V5-tagged rat L1 ORF2 plasmid transfection, fluorescein labelled Alu RNA was transfected into Oryzomys palustris RPE cells. Cells were fixed in 4% paraformaldehyde, and L1 ORF2p were detected using anti-VS (1:5000, Catalogue #600-442-378, Rockland Immunochemicals, Inc., Limerick, Pa., United States of America). For Alu cDNA and L1 ORF2p co-localization, after 48 hours of V5-tagged rat L1 ORF2 plasmid transfection, Alu RNA was transfected into Oryzomys palustris RPE cells. Alu cDNA was monitored by in situ hybridization and L1 ORF2p was detected using anti-V5 antibody. RPE65 and Alu cDNA in human tissue were monitored by in situ hybridization staining of Alu cDNA followed by immunostaining with anti-human RPE65 antibody conjugated with DYLIGHT™ 488 (1:250; Thermo Fisher Scientific Catalogue # MA5-16042). Slides were mounted in PROLONG™ Gold (Thermo Fisher Scientific) and images were acquired using a MR Nikon confocal microscope system.

Pull-down assays. To monitor the association of L1 ORF2p with Alu RNA and Alu cDNA, V5-tagged L1 ORF2p expressing RNaseH-deficient HeLa cells (Mackenzie et al., 2016) (gift of A. P. Jackson and M. A. Reijns, MRC Human Genetics Unit, MRC Institute of Genetics and Molecular Medicine, The University of Edinburgh, Edinburgh, United Kingdom) transfected with biotinylated Alu RNA (vs. mock) were utilized. Briefly, biotinylated Alu RNA-transfected cells were crosslinked with 1% formaldehyde for 15 minutes at room temperature and lysed to collect cytosolic fractions. Streptavidin Dynabeads blocked with 1% BSA (for 18 hours) were incubated with the cytosolic lysates diluted in BC200 buffer (20 mM HEPES, pH 7.9, 0.2 mM EDTA, 0.5 mM DTT, 20% glycerol, 0.2% NP-40, and 200 mM KCl) for 2 hours at 4° C. The beads were then washed twice with BC200, heated at 95° C. for 30 minutes. The pull-down samples were subsequently analyzed by immunoblotting with anti-VS antibody.

Using the same experimental system, a reverse pulldown assay was performed to detect the presence of Alu RNA and Alu cDNA in the V5-L1 ORF2p immunoprecipitate. Briefly, cytosolic lysates (1 mg at 500 ng/μl) in BC200 buffer, prepared as above, were precleared by incubating with beads for 6 hours. Precleared cytosolic lysates were subjected to immunoprecipitation using 15 μg of anti-VS antibody for overnight at 4° C. The immune complexes were captured by incubation with pre-blocked beads (4 hours at 4° C.). The beads-captured immune complexes were washed twice with BC200. Finally, the beads-captured immune complexes were resuspended in either 1 ml of Trizol reagent followed by RNA purification (for detecting Alu RNA) or subjected to Proteinase K treatment and reverse crosslinking (overnight) followed by ethanol precipitation of DNA (for detecting Alu cDNA). The Alu RNA was detected by direct blotting of biotinylated Alu RNA. DNA purified from these assays was analyzed by equator blotting to detect Alu cDNA.

Read alignment and Alu expression analysis. Custom sequence analysis code was written in Python 3.6 and R 3.4.4. Read alignment and feature mapping were executed with STAR 2.5.3a and featureCounts 1.6.1, respectively. In the first step of the pipeline, reads from raw FASTQ files were aligned to the annotated human genome hg19 using alignment software STAR with the following command line call:

./STAR-runThreadN 16-genomeDir/path/to/STAR/genome-outFilterMultimapNmax 40--readFilesIn/path/to/FASTQ/file.fq
The outFilterMultimapNmax parameter in the command line above allows us to keep multi-aligned reads with up to 40 alignments. Taking this file, as well as a GTF file storing all annotated, human Alu loci, as input, the program featureCounts was adopted to identify reads that are uniquely mapped to each Alu locus:
./FC-R SAM-T 16-t exon-g gene_id-a/path/to/GTF/alu.gtf-o/path/to/output/Alu.counts/path/to/alignments/STARAlignments.sam

The uniquely aligned Alu reads were then used to quantify gene-level Alu expression to understand whether a gene, such as RPE-specific, was enriched in Alu expression. The featureCounts program was again adopted, taking the gene gtf as well as the identified Alu reads as input.

Beyond uniquely mapped reads (those having tag ‘NH:i:1’ in the STAR alignment SAM file), additional analysis of the multi-aligned reads that were family-specific in the Alu expression quantification was performed. This was done by a script built in-house. For each read that was multi-mapped, the script iterated through all of its mapped Alu locations and determined whether it was mapped to a single family or multiple families. Our data contained about 325k uniquely aligned Alu reads and about 25k multi-aligned reads that were family-specific. The overall distribution of family-level Alu abundances taking into account family-specific reads in addition to uniquely mapped reads were plotted. To explore whether reads could be uniquely mapped to the young AluY family, we collected reads that were family-specific to individual AluY subfamily and also identified reads that were multi-mapped to both the AluY family and at least one of the young Alu subfamilies. In total, there were 2,549 family-specific young AluY reads and 1,655 reads that could be mapped to both AluY and its young Alu families.

Code Availability. Example scripts illustrating the sequence analysis process are publicly hosted on the World Wide Web at https://github.com/ElkHairCaddis/Alu.

Statistical analysis. The binary readouts of RPE degeneration (i.e., the presence or absence of RPE degeneration on fundus and ZO-1-stained flat-mount images) were analysed with Fisher's exact test. Cell-morphometry data were assessed with two-tailed Mann-Whitney U test. All other data were expressed as means±s.e.m. and were analysed with Mann-Whitney U test or one-way ANOVA with Bonferroni's post-hoc test. P values <0.05 were deemed statistically significant. Sample sizes were selected on the basis of power analysis α=5%; 1−β=80%, such that we were able to detect a minimum of 50% change, assuming a sample s.d. based on Bayesian inference. Outliers were assessed with Grubbs' test. On the basis of this analysis, no outliers were detected, and no data were excluded. Fewer than 5% of subretinal-injection recipient tissues were excluded on the basis of predetermined exclusion criteria (including haemorrhage and animal death due to anaesthesia complications) relating to the technical challenges of this delicate procedure.

Health Records Database Analyses.

Source 1

This study used claims from the VA system from January 2000-July 2017. Data were extracted from the VA Informatics and Computing Infrastructure (VINCI). Data include all inpatient, outpatient and pharmacy claims. The completeness, utility, accuracy, validity, and access methods are described on the VA website, http://www.virec.research.va.gov.

Source 2

This study used claims from the PearlDiver database, which contains patient records obtained from analysis of commercial insurance claims from Humana health insurance beneficiaries over the time-period 2007-2016.

Participants and Sample Selection. Patients were included in the analysis if they met these criteria: had at least 2 diagnoses of HIV/AIDS during the study. Individuals with pre-existing atrophic (“dry”) age related macular degeneration (>1 diagnosis during lookback) were excluded as were individuals who were younger than age 50.

Exposure to Different Classes of Medications to Treat HIV/AIDS. Individuals were classified as receiving NRTI if they filled >1 outpatient pharmacy prescription for these medications as identified based on American Hospital Formulary Service drug codes. Please see Table 1 below for a list of specific medications in the 3 classes (NRTI, NNRTI, protease inhibitors (PI)). Use of combination medications (Efavirenz/Tenofovir disoproxil fumarate/emtricitabine) were counted as taking medications from each class. Exposure to NRTI medication was the key predictor and was set as 1 if a patient had any exposure to an NRTI during the study and 0 otherwise.

TABLE 1
List of Medications Studied
Nucleoside reverse-transcriptase inhibitors (NRTIs)

Abacavir; Abacavir/Dolutegravir/Lamivudine; Abacavir/Lamivudine;
Abacavir/Lamivudine/Zidovudine; Adefovir; Cobicistat/Elvitegravir/
Emtricitabine/Tenofovir; Didanosine; Efavirenz/Emtricitabine/Tenofovir;
Emtricitabine; Emtricitabine/Rilpivirine/Tenofovir; Emtricitabine/
Tenofovir; Entecavir; Lamivudine; Lamivudine/Zidovudine; Stavudine;
Tenofovir; Zidovudine

Nonnucleoside reverse-transcriptase inhibitors (NNRTIs)

Efavirenz; Efavirenz/Emtricitabine/Tenofovir; Emtricitabine/Rilpivirine/
Tenofovir; Etravirine; Nevirapine; Rilpivirine

Protease inhibitors (PIs)

Atazanavir; Darunavir; Fosamprenavir; Lopinavir/Ritonavir; Ritonavir;
Saquinavir; Tipranavir
*Combination drugs are listed in each component drug's category

Dependent Variable. Time to initial diagnosis of dry macular degeneration during follow-up period, as identified by ICD-9-CM code 362.51 and ICD-10-CM code H35.31, was the dependent variable for this analysis. Patients with dry macular degeneration during lookback were excluded. Observation of beneficiaries was right censored at study end Jul. 1, 2017 (VA) or Dec. 31, 2016 (PearlDiver).

Analyses. To analyse the risk of dry macular degeneration between those exposed to NRTI and those not exposed to NRTI medication, we fit adjusted and unadjusted Cox proportional hazard models. The adjusted model included covariates: age, gender, race, body-mass index (BMI), tobacco use, NNRTI use, PI use, viral load, CD4 counts, and Charlson Comorbidity Index score.

Data Availability. Sequencing data have been deposited in the Gene Expression Omnibus (GEO) public functional genomics data repository of the United States National Center for Biotechnology Information (NCBI) under Accession. No. GSE120338. All other relevant data that support the findings of this study are available from the co-inventors upon reasonable request.

### Example 1

### Alu cDNA in Human GA

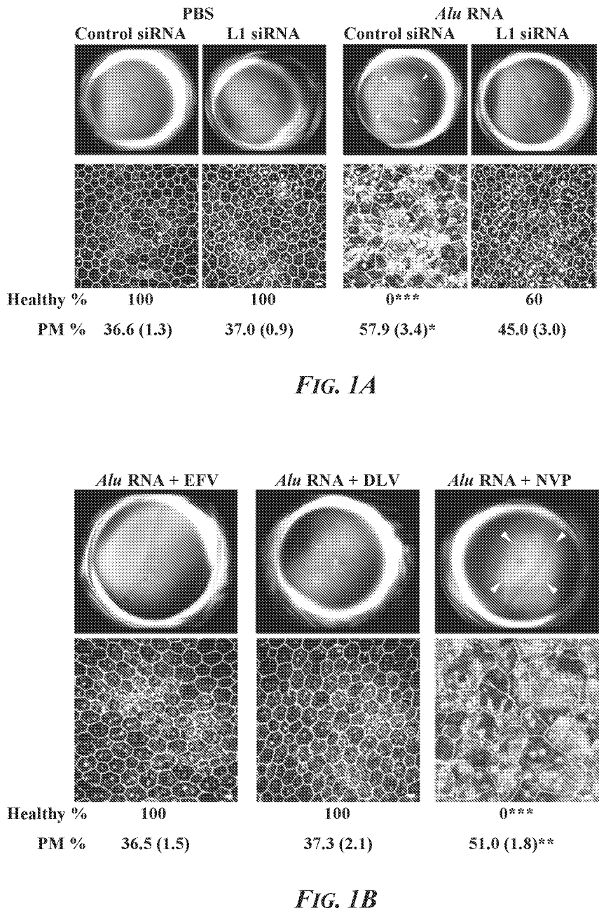

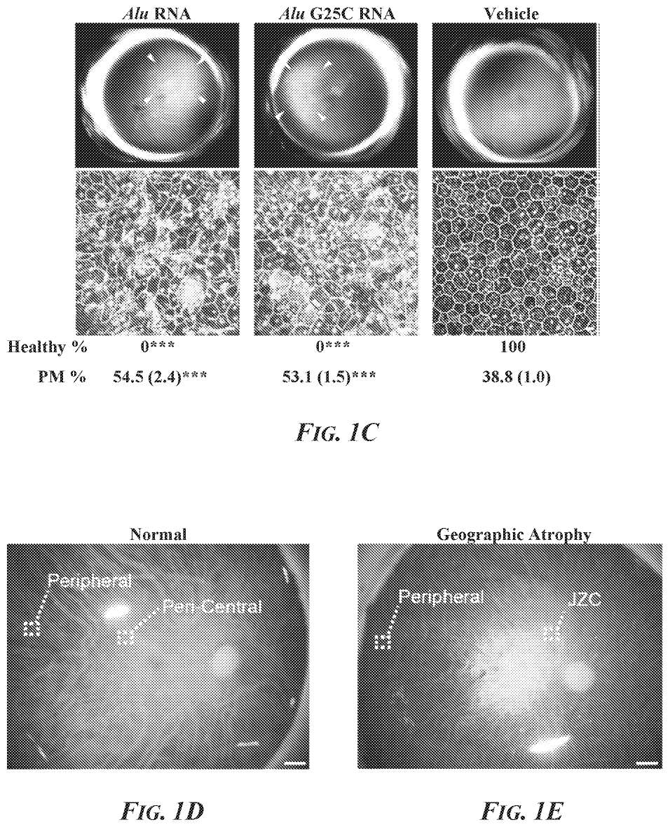

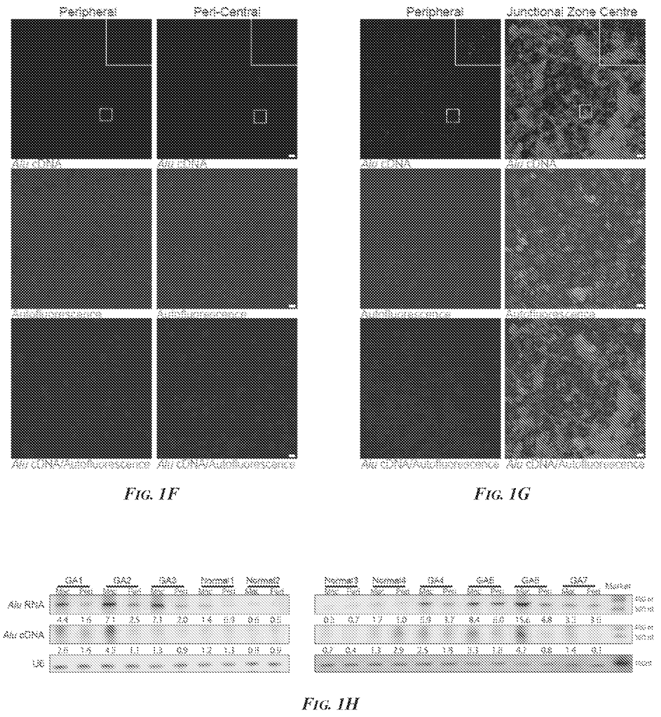

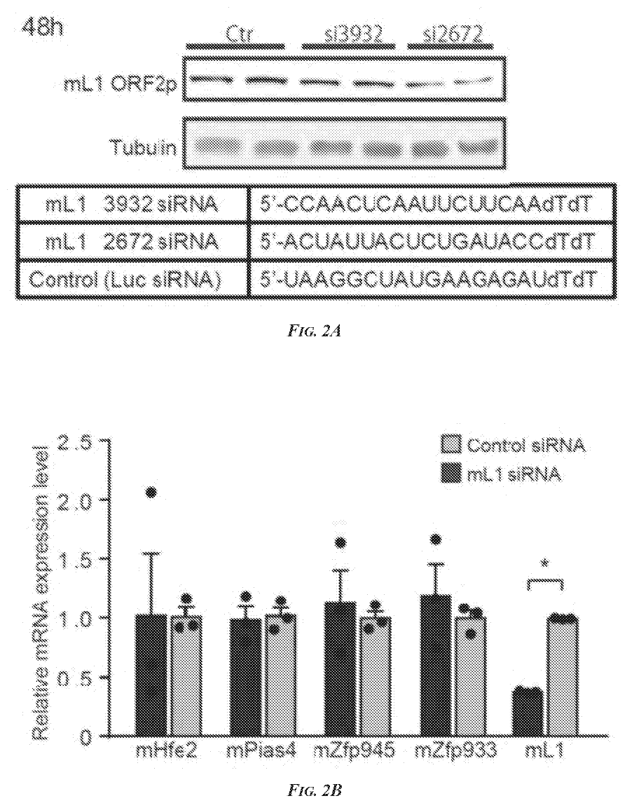

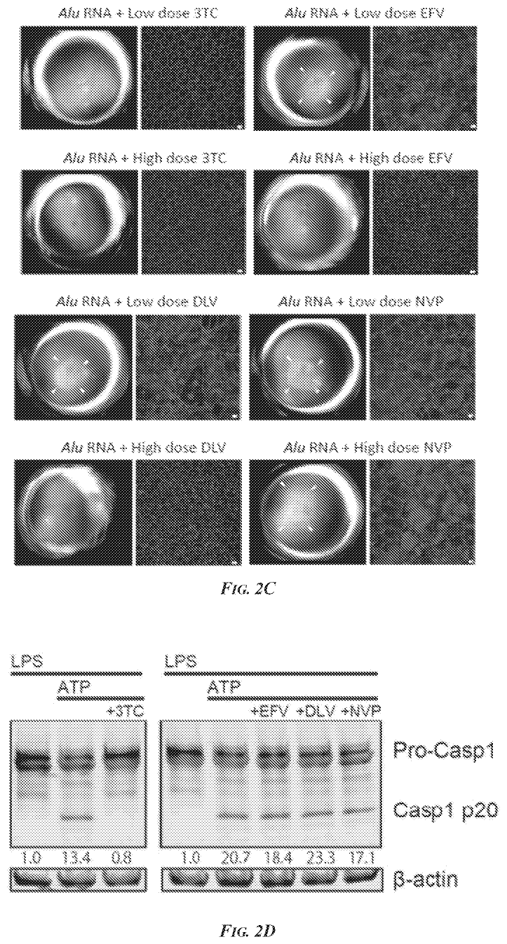

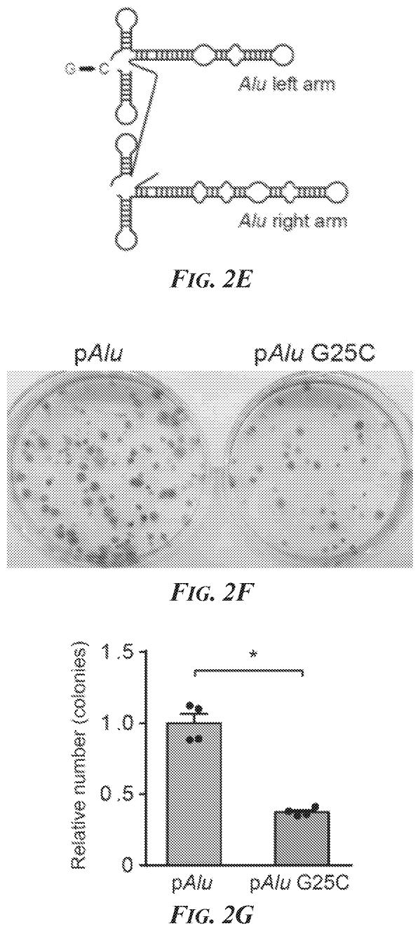

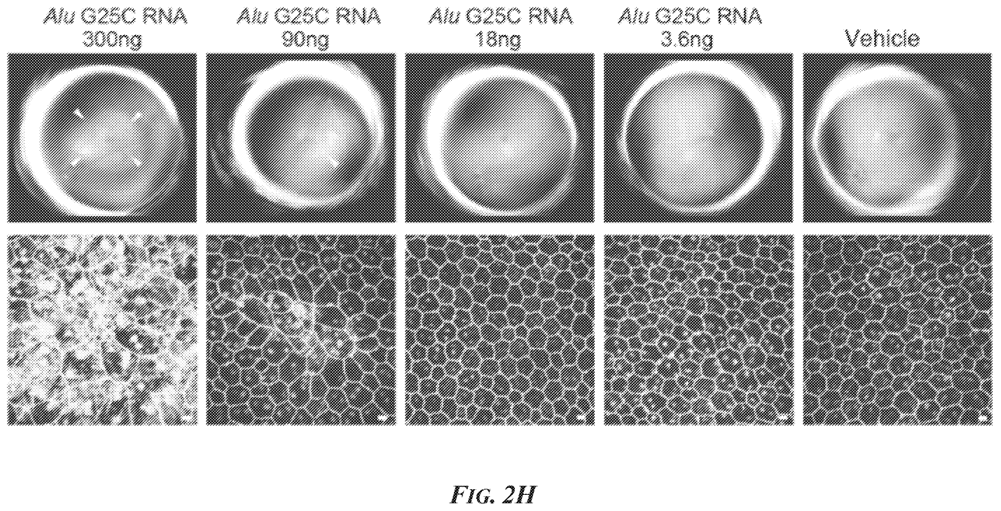

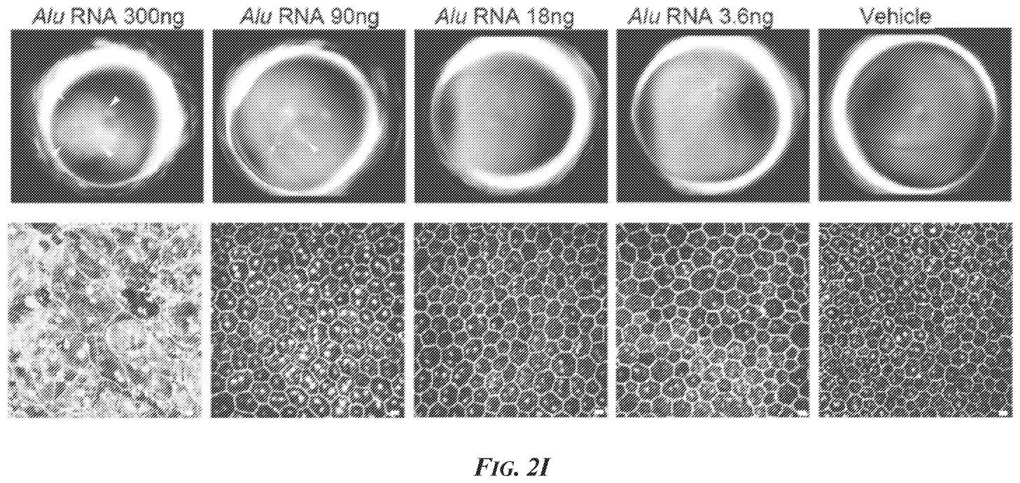

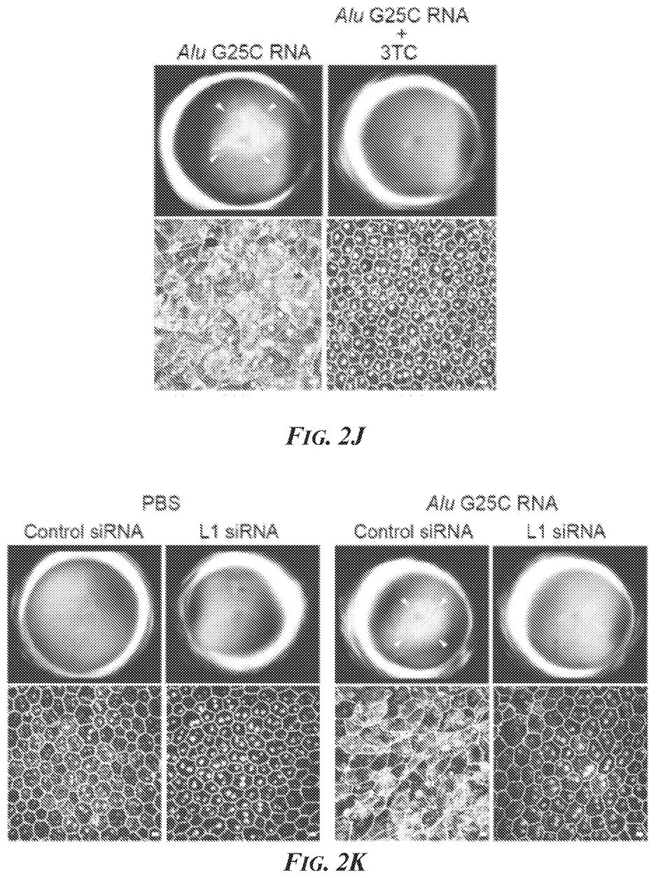

Previously, it was demonstrated that NRTIs have two distinct activities: (1) inhibition of reverse transcriptase and (2) inhibition of the NLRP3 inflammasome (Fowler et al., 2014). While the reverse transcriptase inhibitory function was dispensable for the anti-inflammatory effects of NRTIs, we did not directly test whether reverse transcription of Alu RNA mediated its toxicity. To do so, we first examined whether endogenous reverse transcriptase mediated Alu RNA toxicity. L1 has active endogenous reverse transcriptase activity that acts on Alu RNA1. Therefore, we tested whether antagonizing L1 affected Alu RNA-induced toxicity. We found that subretinal delivery of a cell-permeable, nonimmunogenic cholesterol-conjugated siRNA (17+2-nt in length; SEQ ID NOs: 48 and 49; Kleinman et al., 2008; Kleinman et al., 2012) targeting L1 prevented Alu RNA-induced RPE degeneration in wild-type mice (FIGS. 1A, 2A, and 2B). We then tested the non-nucleoside reverse transcriptase inhibitors (NNRTIs) efavirenz (EFV) and delaviridine (DLV), which inhibit L1 reverse transcriptase at high doses (Merluzzi et al., 1990; Dai et al., 2011). Intravitreous administration of EFV and DLV did not block Alu RNA-induced RPE degeneration at low doses equimolar to the previously determined (Fowler et al., 2014) effective concentration of the NRTI 3TC ((−)-b-L-2′,3′-dideoxy-3′-thiacytidine; FIG. 2C); however, at high doses, EFV and DLV blocked Alu RNA toxicity (FIGS. 1B and 2C) even though they did not inhibit NLRP3 inflammasome activation by lipopolysaccharide (LPS) and ATP stimulation (FIG. 2D). In contrast, the NNRTI nevirapine (NVP), which does not inhibit L1 reverse transcriptase (Merluzzi et al., 1990; Dai et al., 2011) or NLRP3 inflammasome activation (FIG. 2D), did not prevent Alu RNA-induced RPE degeneration at low or high doses (FIGS. 1B and 2C). These results support the concept that endogenous L1 reverse transcriptase activity per se mediates Alu RNA toxicity.

Next, we determined whether Alu RNA toxicity was mediated by retrotransposition, a process that couples L1 reverse transcription with genomic integration of Alu cDNA. To decouple reverse transcription from genomic integration, we synthesized a mutant Alu with a G25C mutation in the left arm monomer (FIG. 2E). The design of this mutant was based on a previous finding that a G25C loss-of-interaction mutation within the SRP9/14 RNP binding site of Alu greatly reduced its retrotransposition ability (Chang et al., 1997; Bennett et al., 2008). Despite its retrotransposition deficiency (FIGS. 2F and 2G), Alu G25C RNA was still toxic in wild-type mice (FIG. 1C). Alu G25C RNA induced RPE degeneration in a dose-dependent manner similar to that of Alu RNA (FIGS. 2H and 21). In addition, Alu G25C toxicity was prevented by 3TC (FIG. 2J) or L1 siRNA (FIG. 2K). We interpreted these findings to mean that L1-mediated reverse transcription, but not retrotransposition, was essential for Alu RNA toxicity. As such, we hypothesized that endogenous reverse transcribed Alu complementary DNA (Alu cDNA) that fails to insert into the host genome is a key intermediate in Alu RNA toxicity.

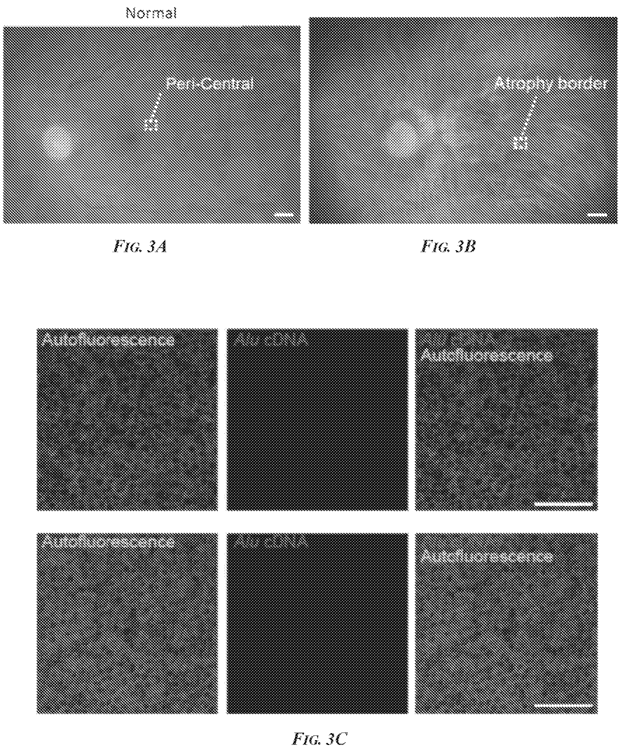

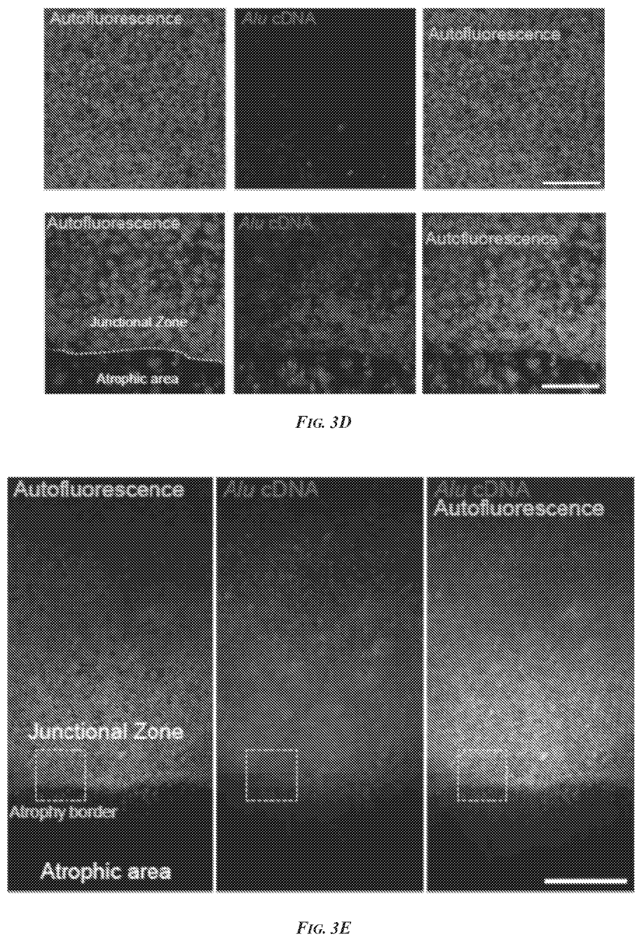

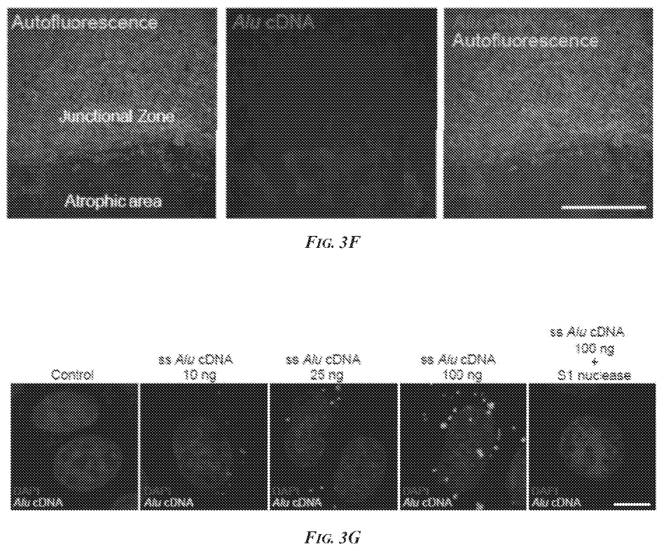

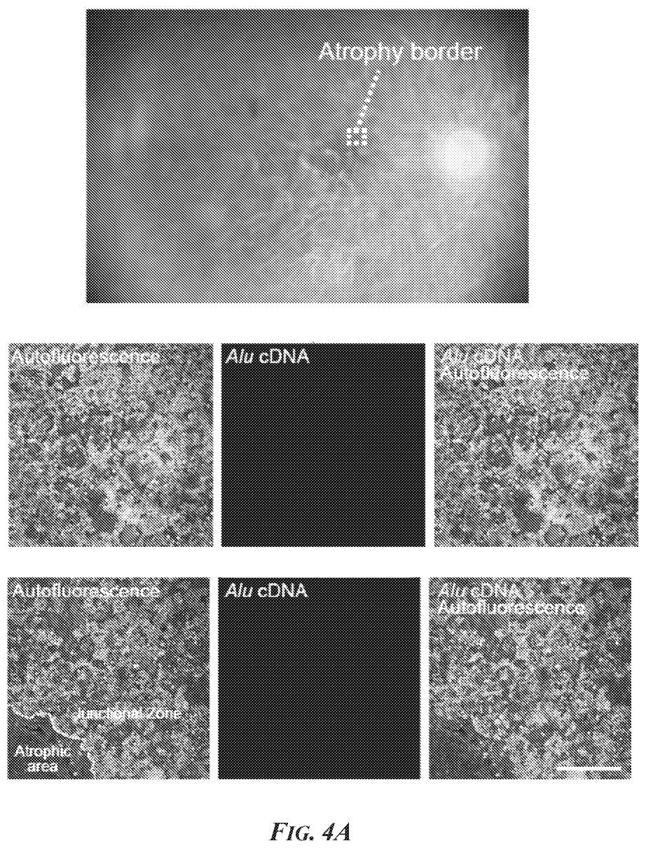

To determine whether endogenous Alu cDNA exists in human eyes with GA, we generated sequence- and strand-specific probes to perform in situ hybridization. GA is topographically heterogeneous within the retina: a junctional zone is interposed between a central area of atrophy and a peripheral area of surviving RPE cells. This metastable region consists of stressed and degenerating RPE cells (Sarks et al., 1988), displays impaired visual function (Hariri et al., 2016), and undergoes atrophy over time as GA expands centrifugally (Holz et al., 2001). In human donor eyes with GA, Alu cDNA was highly enriched at the center of the junctional zone and its border with the atrophic area (FIGS. 1D-1G and 3A-3E). In contrast, Alu cDNA was far less abundant in peripheral disease-free areas of GA eyes and only faintly detected in normal control eyes. The spatial enrichment of Alu cDNA in the most dynamic zone of disease and its paucity in disease-free regions are consistent with the concept that it contributes to GA progression.

To confirm that the detected signals did not represent genomic Alu sequences, we employed a variation of nucleic acid blotting that we term an “equator blot” (a functional combination of northern and Southern blotting), which can be used to detect both RNAs and extrachromosomal DNAs. Using equator blotting, we found that Alu RNA and Alu cDNA levels were increased in the macular RPE of GA eyes (FIG. 1H). As both these species were approximately 300-nt long, they were compatible with bona fide rather than embedded Alu elements.

We performed several quality control steps to confirm the specificity of the in situ hybridization probe for single-stranded Alu cDNA. Treatment with Si nuclease eliminated the Alu cDNA signal, confirming its single-strandedness (FIG. 3F). Samples were also treated with RNase A to avoid detection of RNA. We confirmed, in primary human RPE cells, that this probe detected an artificially synthesized Alu single-stranded DNA in a dose-dependent manner, and that the signal was abolished upon treatment with S1 nuclease (FIG. 3G).

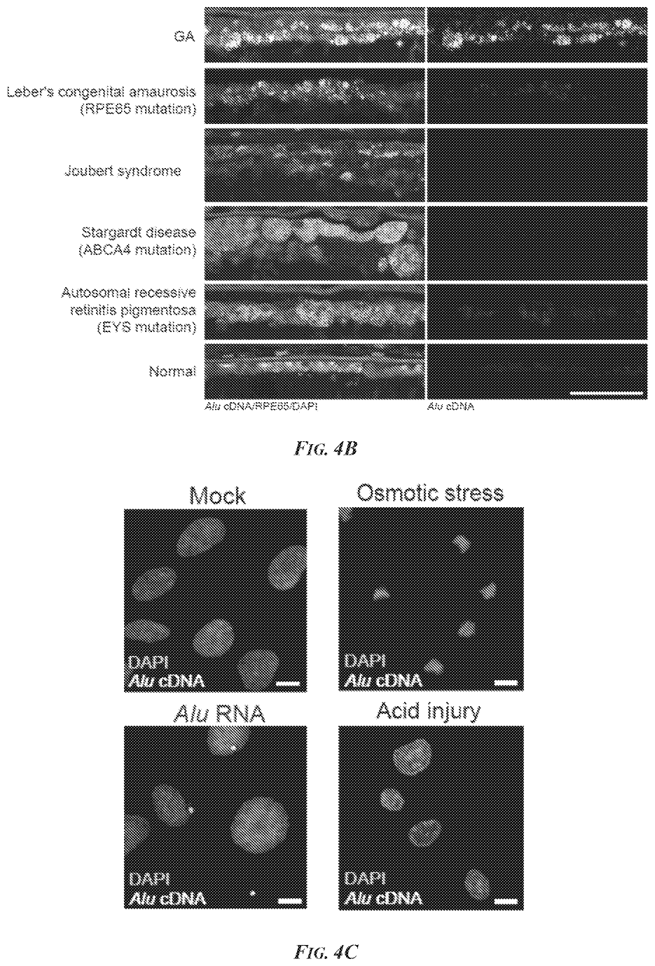

Notably, we did not detect Alu cDNA in an eye with RPE atrophy that developed subsequent to treatment of central retinal vein occlusion with anti-angiogenic drugs (FIG. 4A) nor in the RPE of eyes affected with several other retinal disorders including autosomal recessive retinitis pigmentosa, Joubert syndrome, Leber congenital amaurosis, or Stargardt macular dystrophy (FIG. 4B), suggesting a disease-specificity to the accumulation of Alu cDNA in GA. Furthermore, primary human RPE cells synthesized Alu cDNA when exposed to Alu RNA but not to osmotic stress or acid injury (FIG. 4C), indicating that Alu cDNA generation is not a generic response of dying cells.

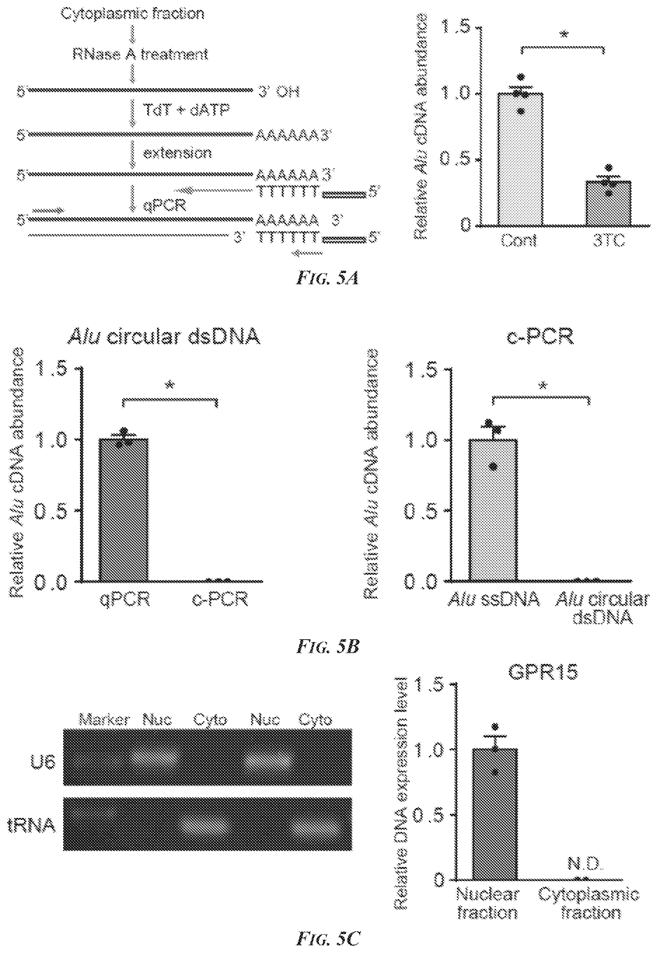

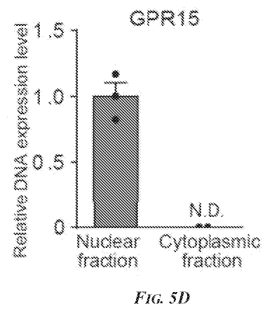

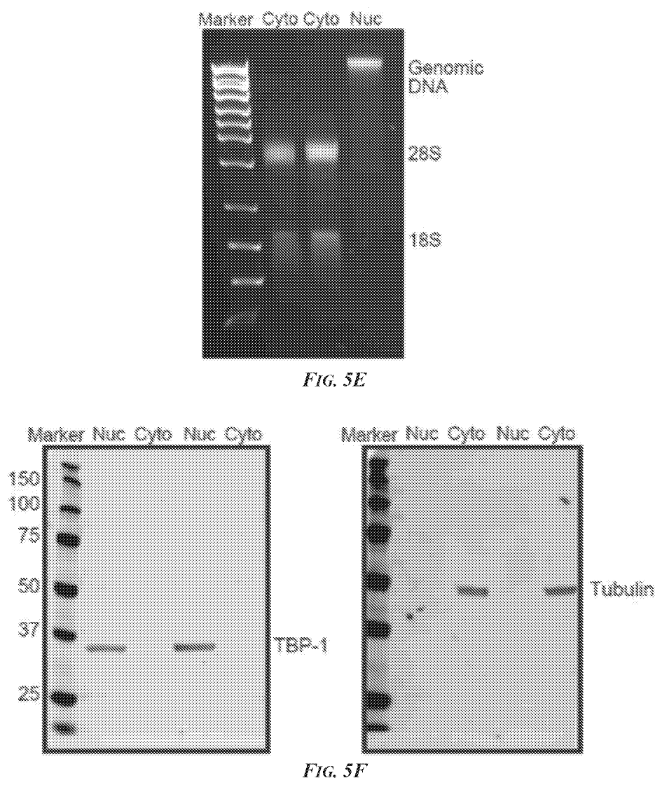

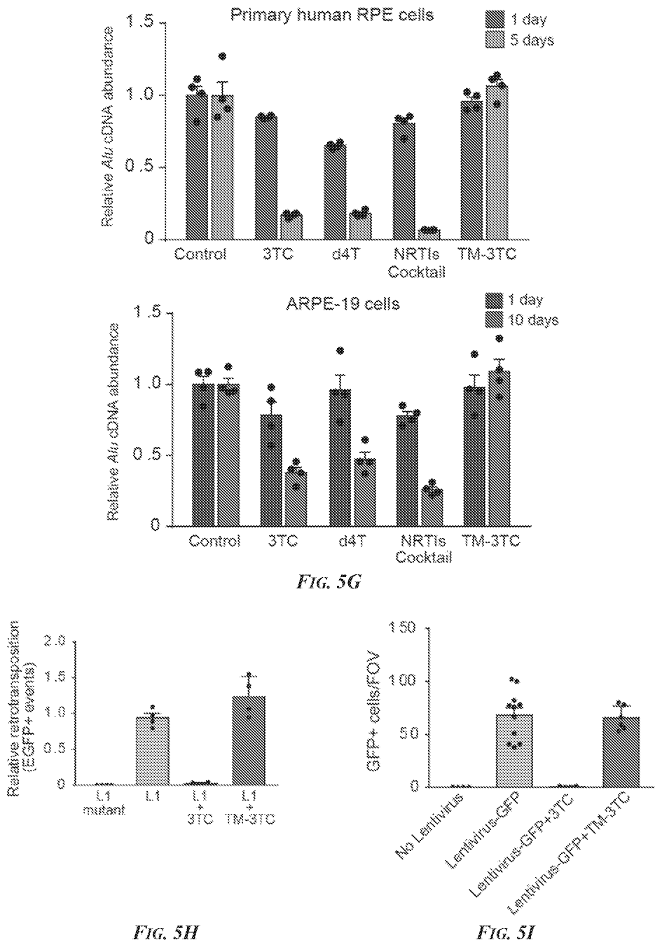

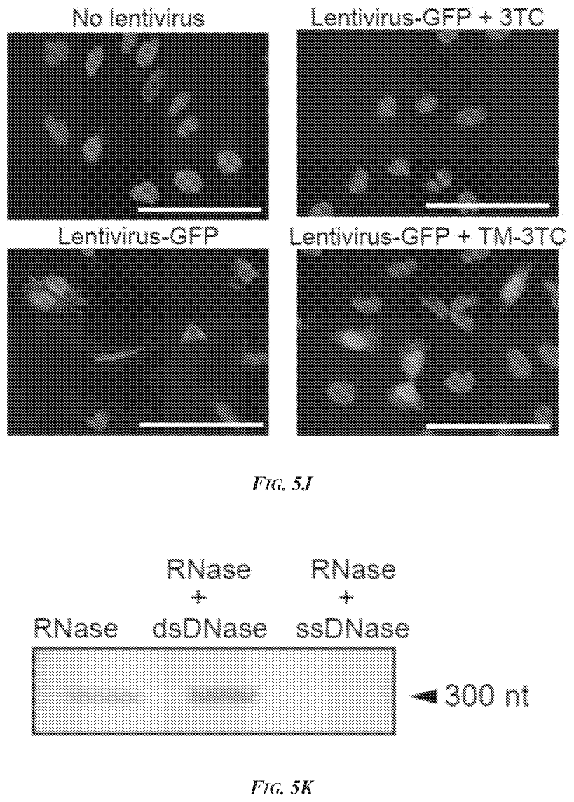

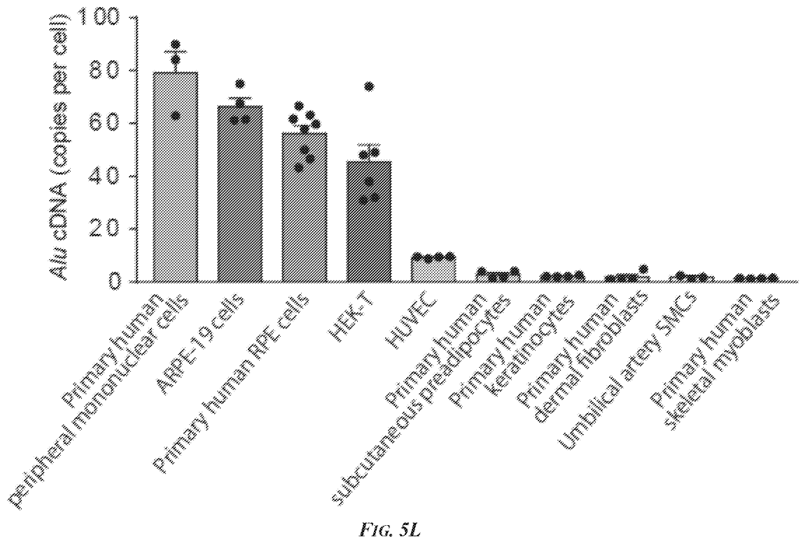

To assess whether Alu RNA was reverse transcribed into Alu cDNA, we subjected cytoplasmic fractions of primary human RPE cells to an adaptor-based PCR quantification method (Alu c-PCR) (FIGS. 5A-5F). Importantly, this method avoids detecting circular forms of extrachromosomal Alu DNAs (FIG. 5B), which are known to be present in the cytoplasm (Krolewski et al., 1984). Exposure to the NRTIs 3TC, d4T (2′,3′-didehydro-2′,3′-dideoxythymidine), or a mixture thereof, dramatically decreased Alu cDNA levels in primary human RPE cells (FIG. 5G). On the other hand, trimethyl-3TC (TM-3TC), an alkyl-modified NRTI derivative that does not inhibit reverse transcriptase (Fowler et al., 2014; Ambati et al., 2018), did not reduce Alu cDNA levels (FIG. 5G). We obtained similar results using ARPE-19 cells, a spontaneously immortalized human RPE cell line (FIG. 5G). As anticipated, 3TC, but not TM-3TC, blocked Alu retrotransposition in a reporter assay (FIGS. 5H-5J). These data indicated that reverse transcription of endogenous Alu RNA led to Alu cDNA expression in human RPE cells. Similar to Alu cDNA from human eyes with GA (FIG. 3F), Alu cDNA in cultured human RPE cells was resistant to RNase A and double-stranded DNase but sensitive to Si nuclease (FIG. 5K).

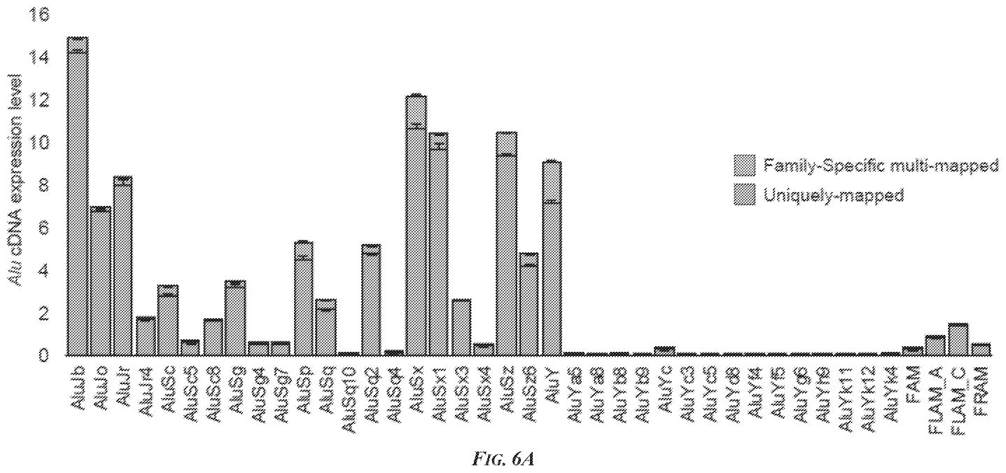

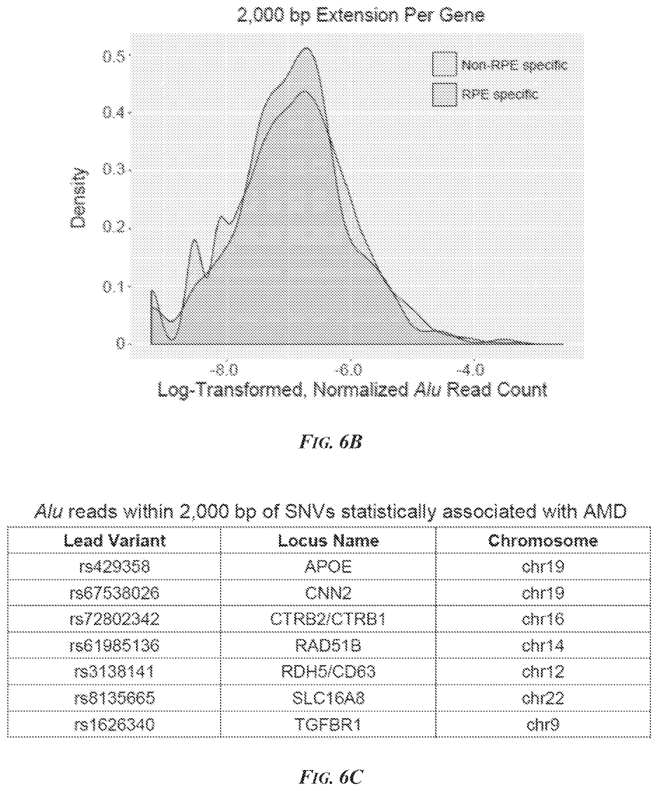

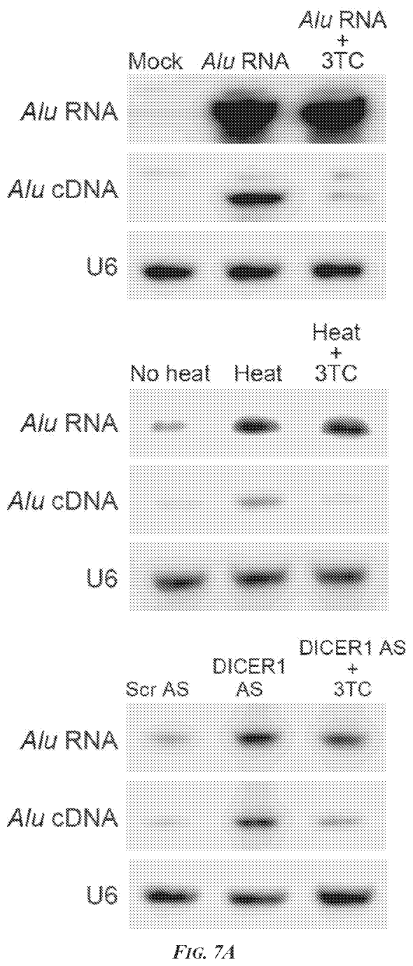

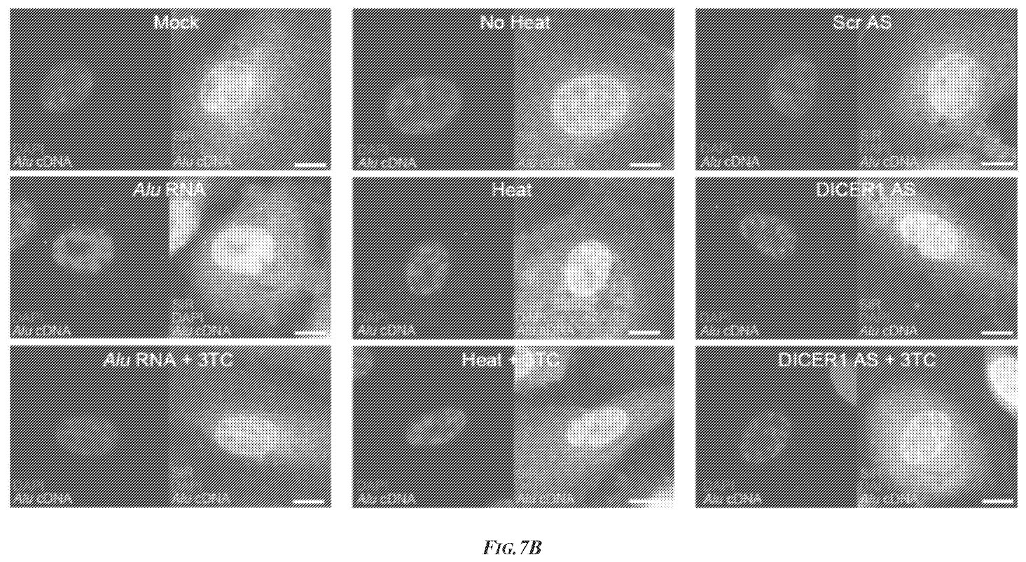

To determine whether Alu cDNA formation was unique to human RPE cells, we investigated its presence in a variety of other human cells using direct amplification by real-time PCR. The basal levels of endogenous Alu cDNA varied more than 50-fold among ten different primary cells and cell lines tested (FIG. 5L). Among the cell types tested, those with the highest expression of endogenous Alu cDNA were primary human peripheral mononuclear cells, ARPE-19 cells, primary human RPE cells, and human embryonic kidney-293-T cells. Since Alus exhibit sequence heterogeneity, we investigated which Alu sequences comprise Alu cDNA. Alu sequences are broadly grouped into J, S, and Y families based on sequence divergence throughout millions of years of genome amplification (Jelinek et al., 1980; Rubin et al., 1980; Jurka & Smith, 1988; Batzer & Deininger, 2002; Mills et al., 2007). We performed next-generation sequencing of cytoplasmic fractions of primary human RPE cells, restricted to 200-800-nt long species to eliminate genomic DNA contamination and embedded Alu elements. We found that Alu S predominated (61% of Alu reads), with lower levels of Alu J (31%) and Alu Y (8%) (FIG. 6A). No significant difference in the overall distribution of these Alu sequences that mapped to RPE-specific (Booij et al., 2010) and non-RPE-specific genes was observed (FIG. 6B). However, we did identify a cluster of Alu sequences within 2,000 bp of 7 single-nucleotide variant loci statistically associated with AMD48 (FIG. 6C).

### Example 2

### Alu cDNA Formation by Reverse Transcription of Alu RNA

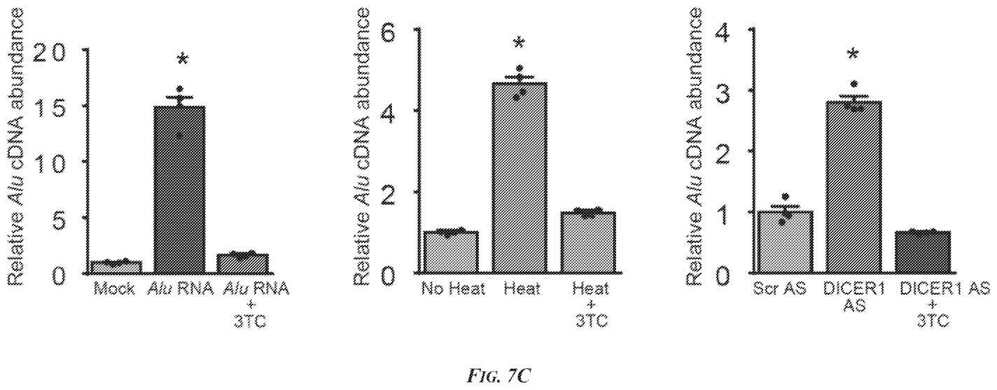

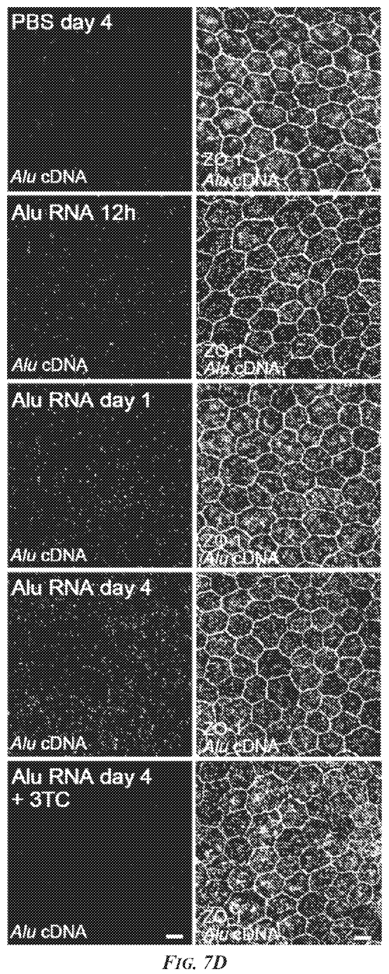

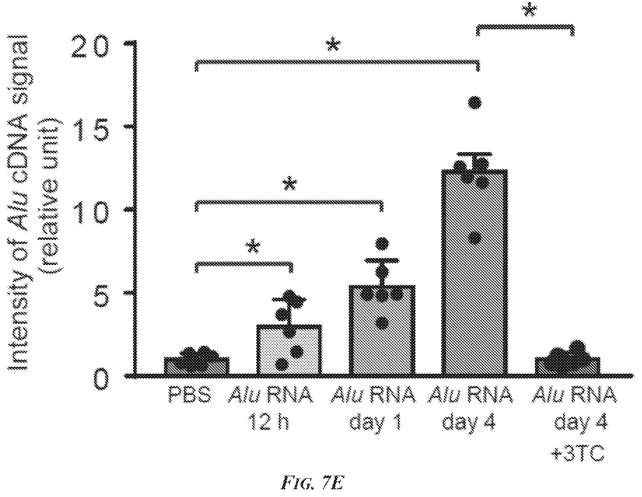

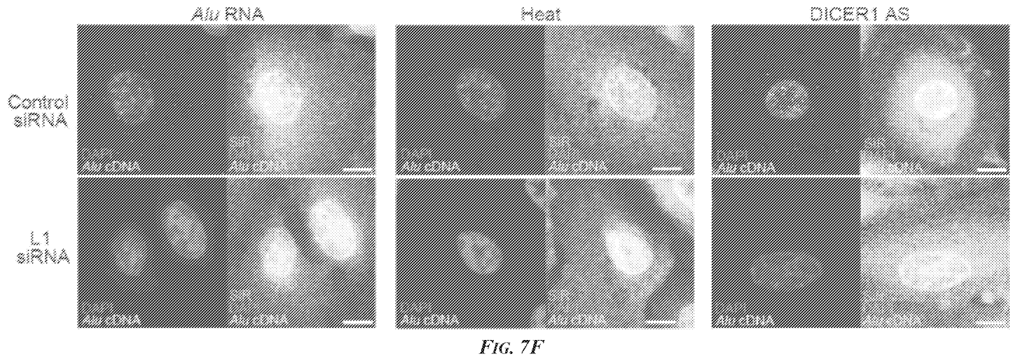

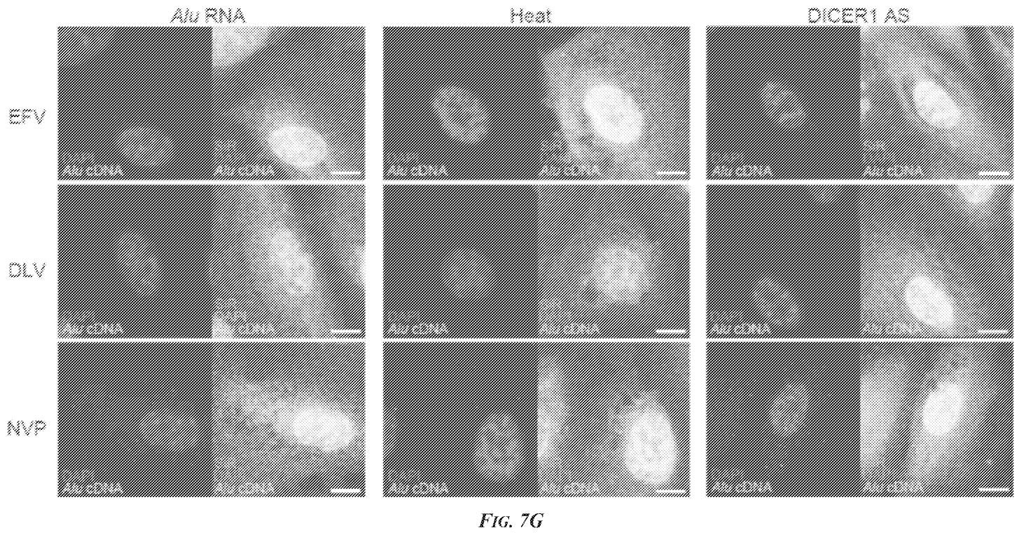

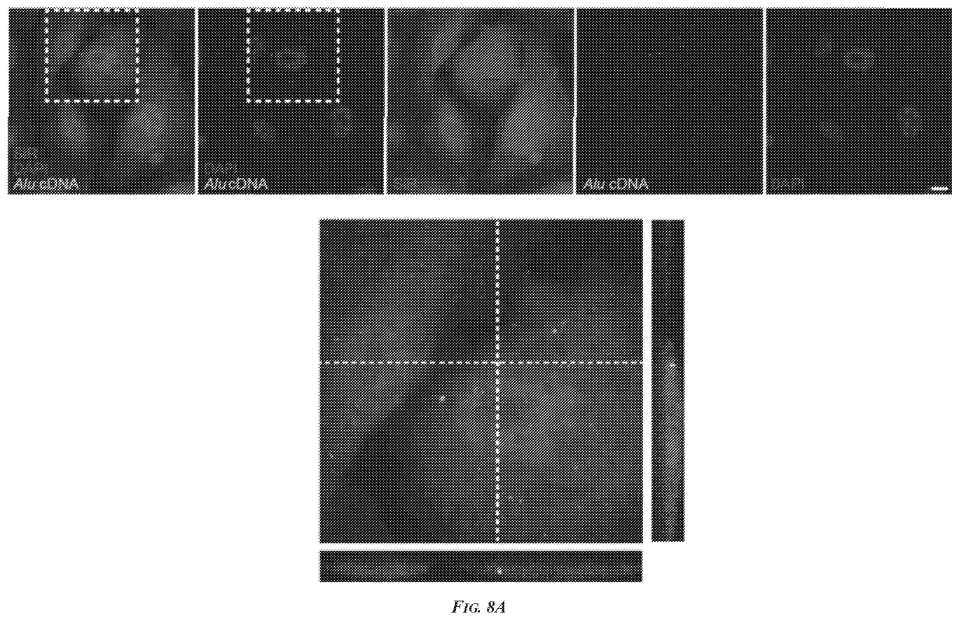

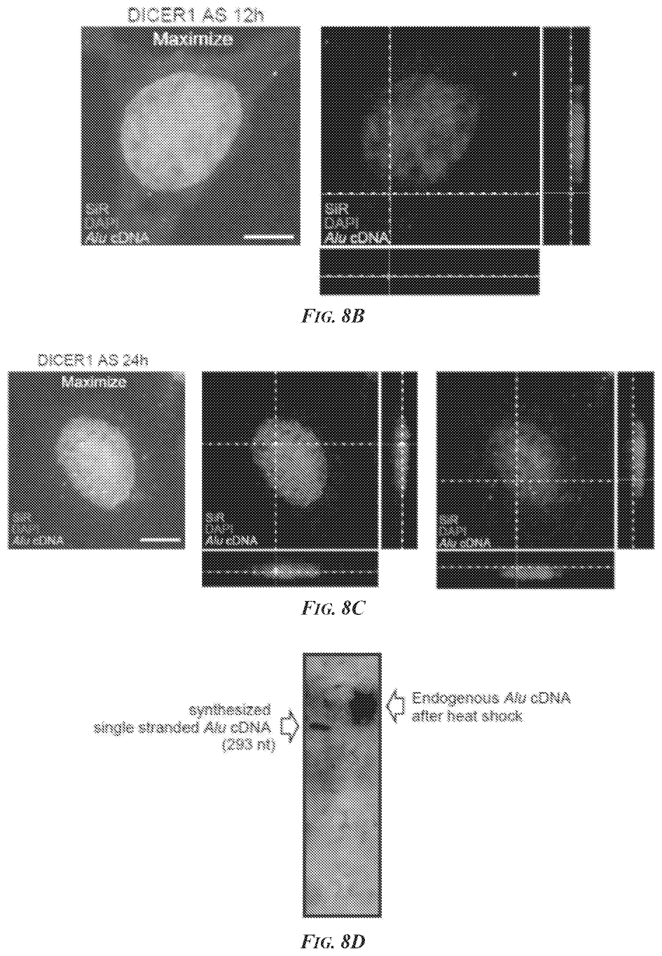

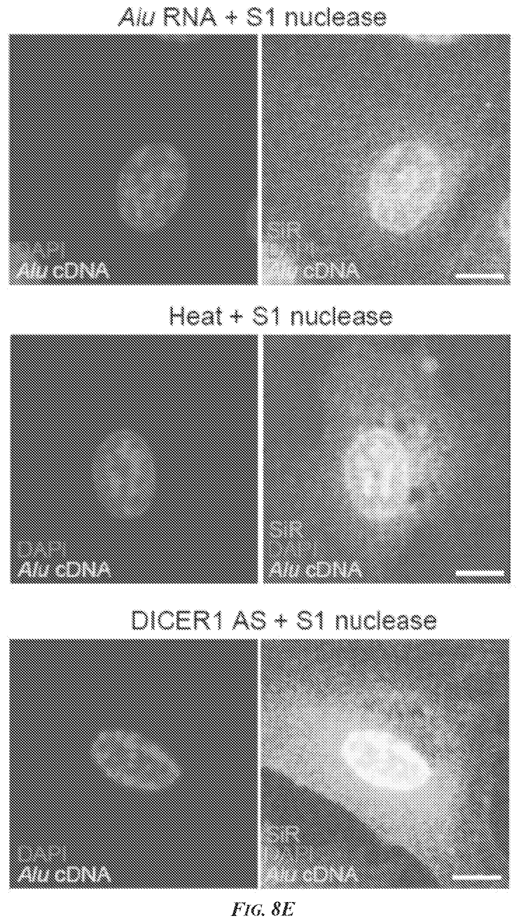

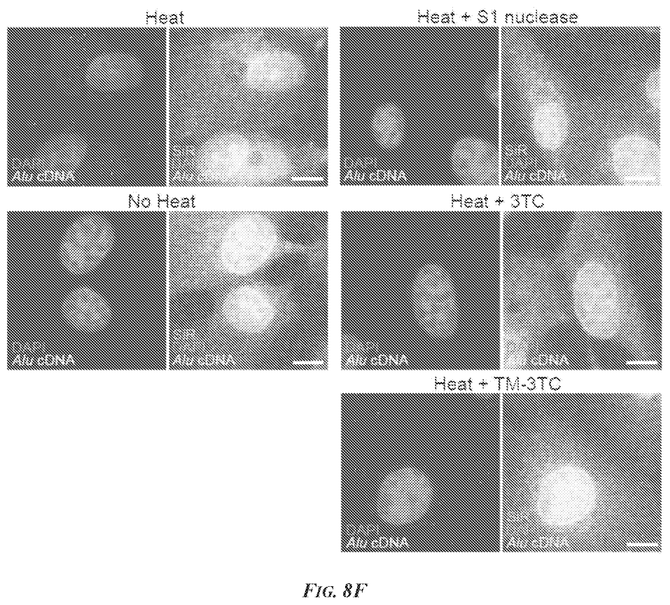

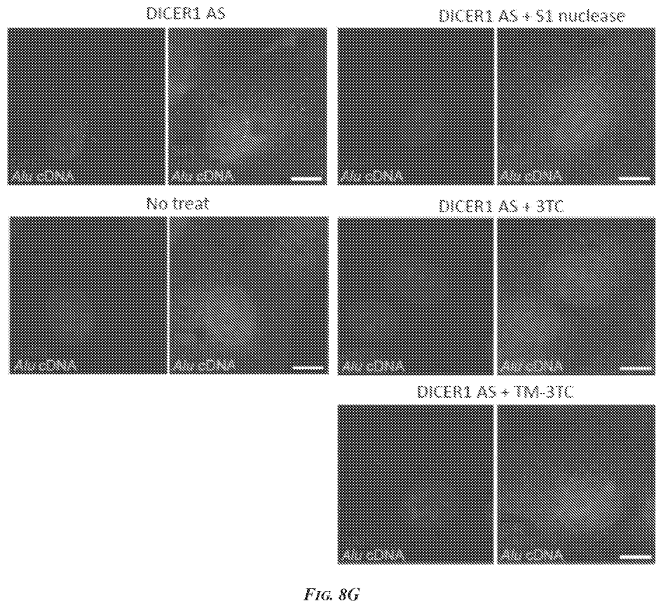

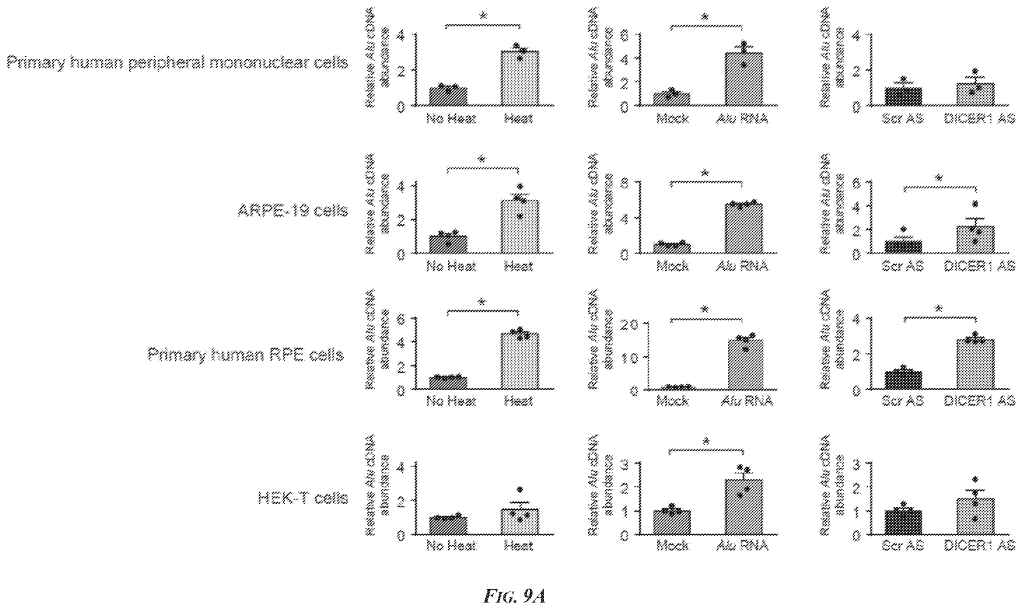

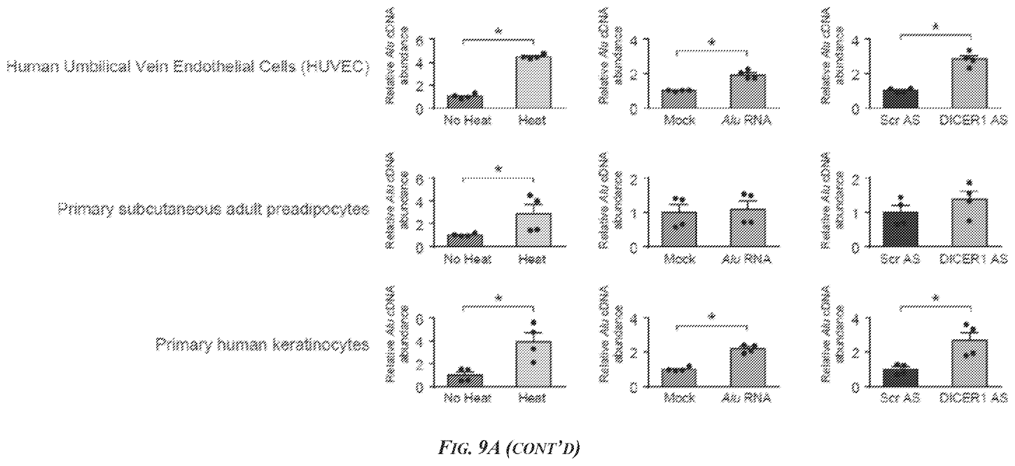

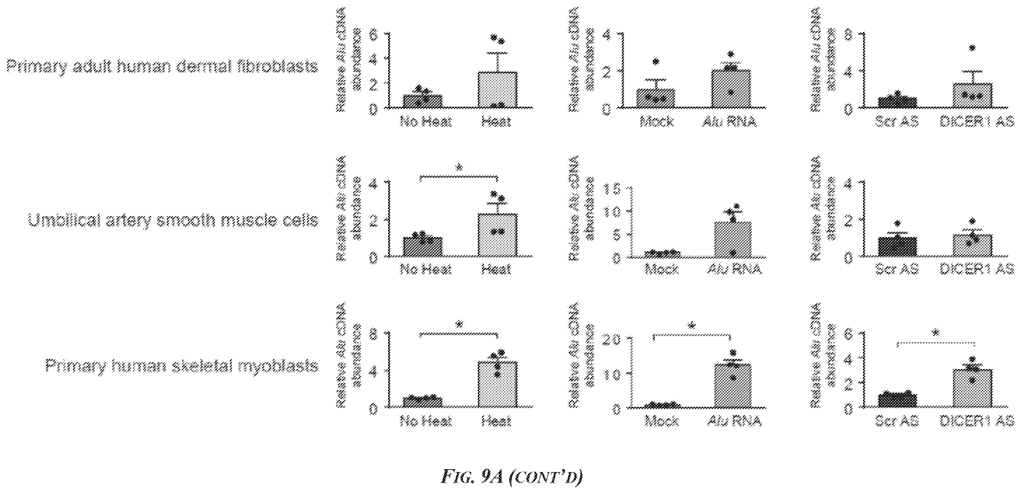

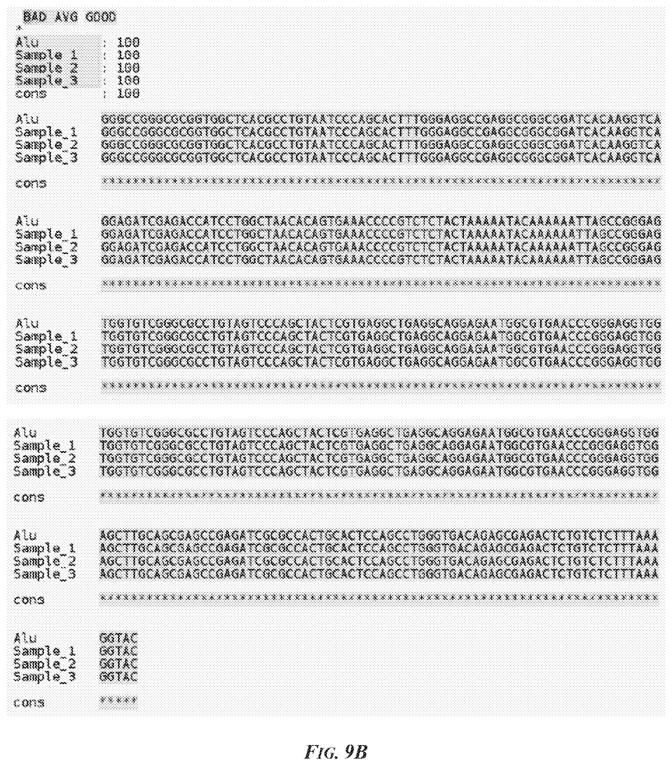

We next assessed whether Alu cDNA expression could be modulated by titrating Alu RNA levels. Using equator blotting, in situ hybridization, and Alu c-PCR, we found that increasing Alu RNA levels by any of three methods (transfection of in vitro transcribed synthetic RNA (Kaneko et al., 2011), heat shock (Liu et al., 1995), or DICER1 knockdown by antisense oligonucleotide; Kaneko et al., 2011) induced Alu cDNA levels, an effect that was abrogated by reverse transcriptase inhibition with 3TC in primary human RPE cells (FIGS. 7A-7C and 8A-8E). Alu cDNA accumulation was predominantly cytoplasmic after all three conditions; however, following DICER1 knockdown, Alu cDNA was also occasionally observed in the nucleus (FIG. 8C). Using in situ hybridization we determined that the increase in Alu cDNA induced by heat shock or DICER1 knockdown in ARPE-19 cells was blocked by 3TC but not by TM-3TC (FIGS. 8F and 8G), confirming the necessity of reverse transcriptase activity. Moreover, treatment with S1 nuclease eliminated the Alu cDNA signal, confirming it as single-stranded DNA (FIG. 8E-8G). Increase of Alu cDNA levels by upregulating Alu RNA was not unique to human RPE cells; we found that Alu cDNA was inducible in ten human cell types by heat shock, Alu RNA transfection, or DICER1 knockdown, albeit to different degrees (FIG. 9A).

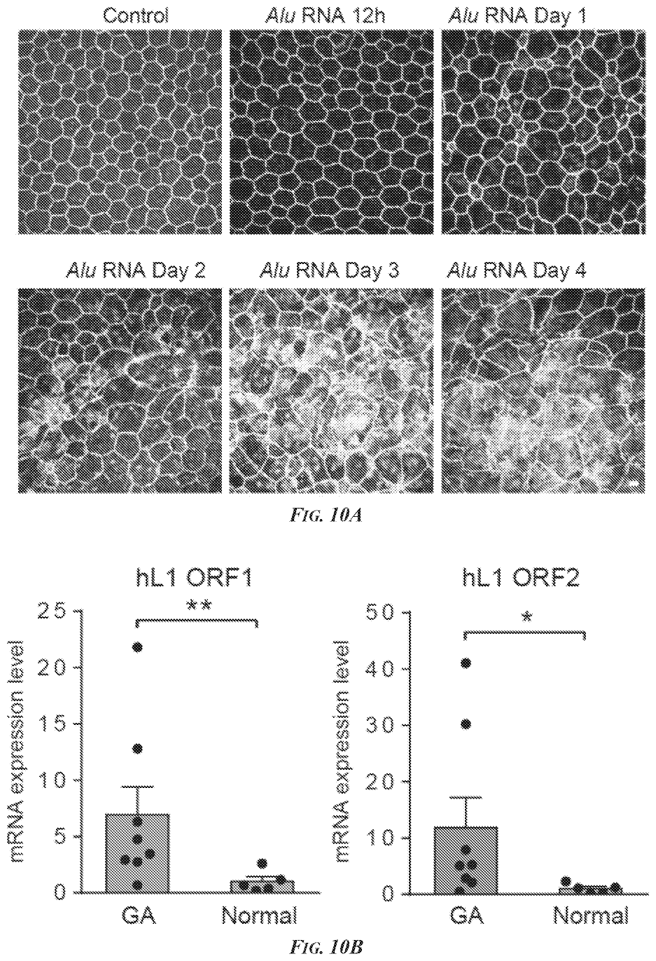

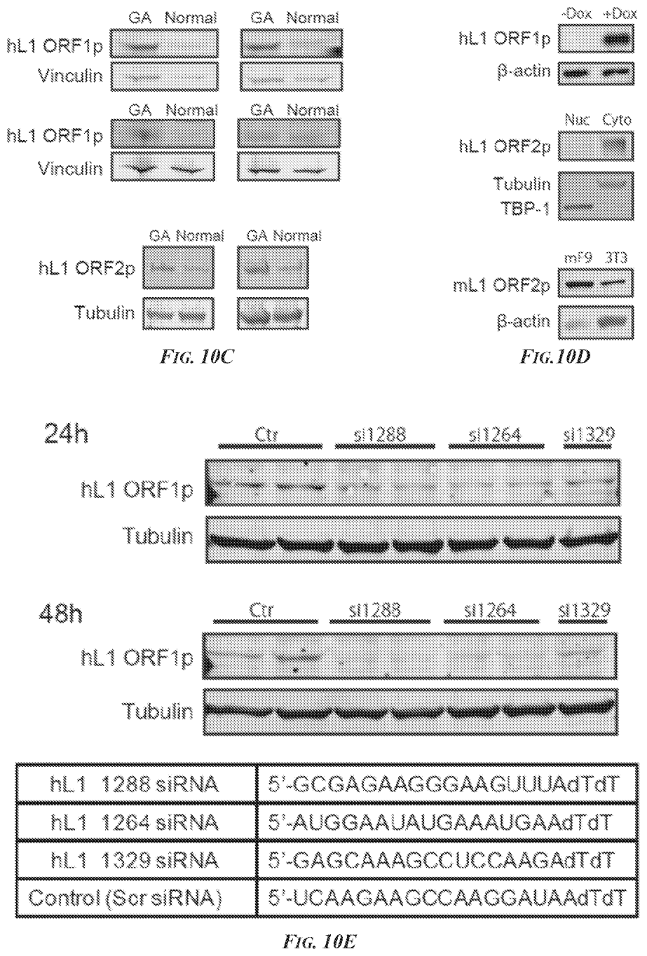

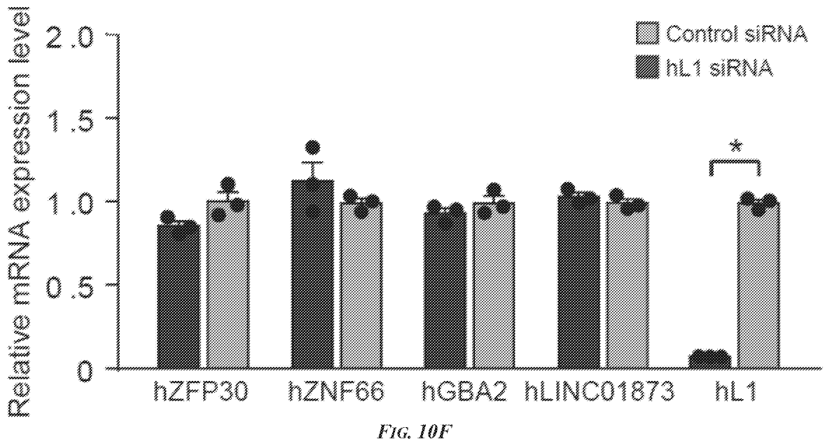

Finally, we directly confirmed the conversion of transfected Alu RNA into Alu cDNA by recipient cells in vitro and in vivo. In mouse fibroblasts, which do not contain contaminating Alu sequences in their genome, we transfected Alu RNA and confirmed via sequencing the presence of Alu cDNA matching the transfected Alu RNA (FIG. 9B). We also tested whether Alu cDNA formation occurred in vivo after subretinal Alu RNA transfection using whole mount in situ hybridization. We performed this study using mice functionally deficient in the inflammasome components caspase-1 and caspase-4 (termed Casp1/4 dko mice), which are protected from Alu RNA-induced RPE degeneration (Tarallo et al., 2012; Kerur et al., 2018), to dissociate Alu cDNA formation from cell death so that signals could be visualized free of distortions arising from degenerating cells. In these mice, Alu cDNA was detected as early as 12 hours after Alu RNA transfection and increased stepwise up to four days later (FIGS. 7D and 7E). Importantly, RPE degeneration was not evident until 1-2 days after Alu RNA transfection, i.e., Alu cDNA formation temporally precedes RPE cell death (FIG. 10A). Notably, 3TC blocked Alu cDNA accumulation, indicating that Alu cDNA production required reverse transcription (FIGS. 7D and 7E).

### Example 3

### L1 Reverse Transcriptase Mediates Alu cDNA Formation

Given the presence of Alu cDNA in human GA eyes, we hypothesized that L1 abundance might also be increased in this disease. Indeed, we found that levels of L1 ORF1 and ORF2 mRNAs and of L1 ORF1p and ORF2p proteins, were enriched in the macular RPE of human GA eyes compared with normal controls (FIGS. 10B-10D).

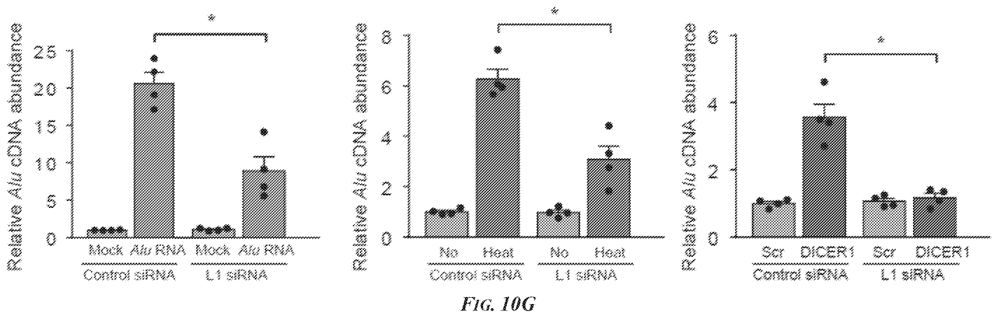

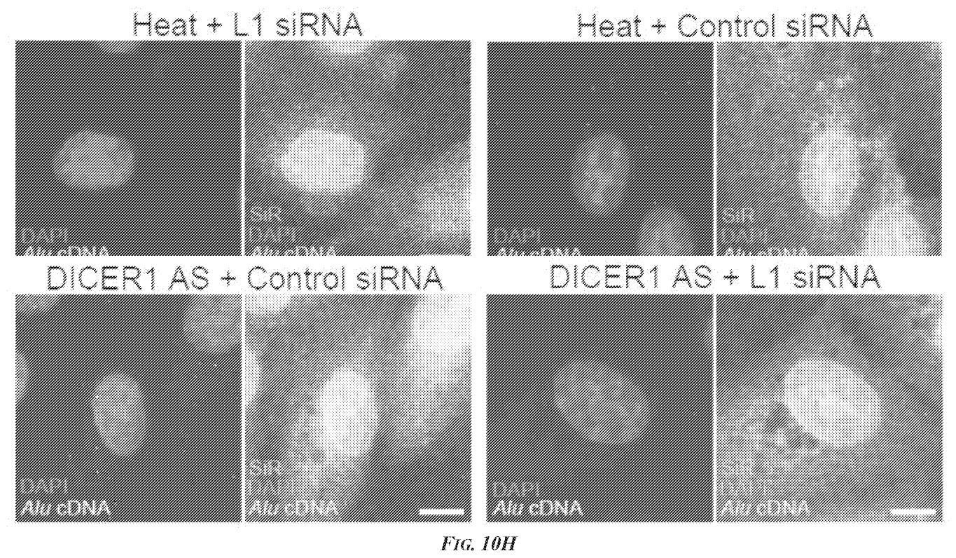

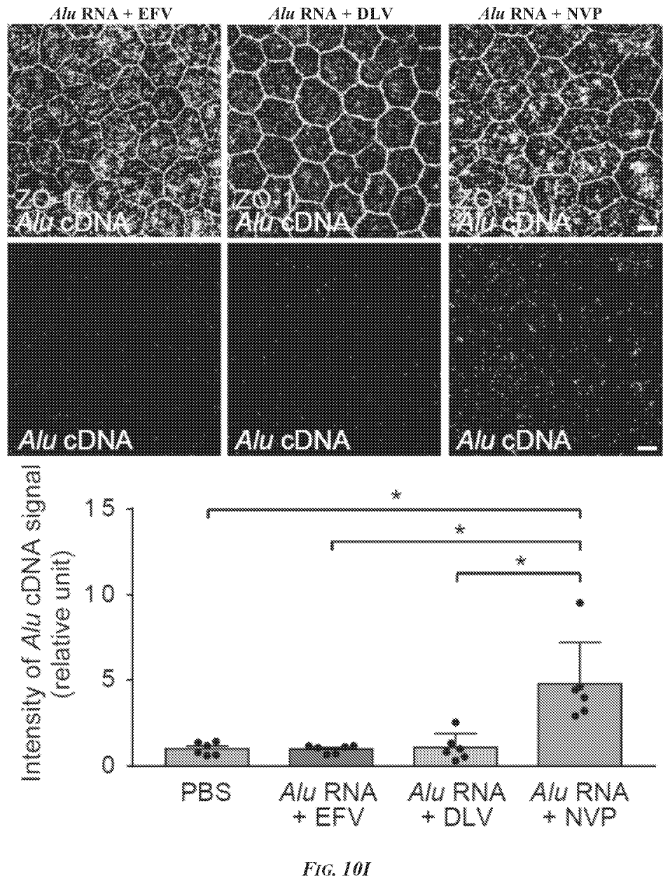

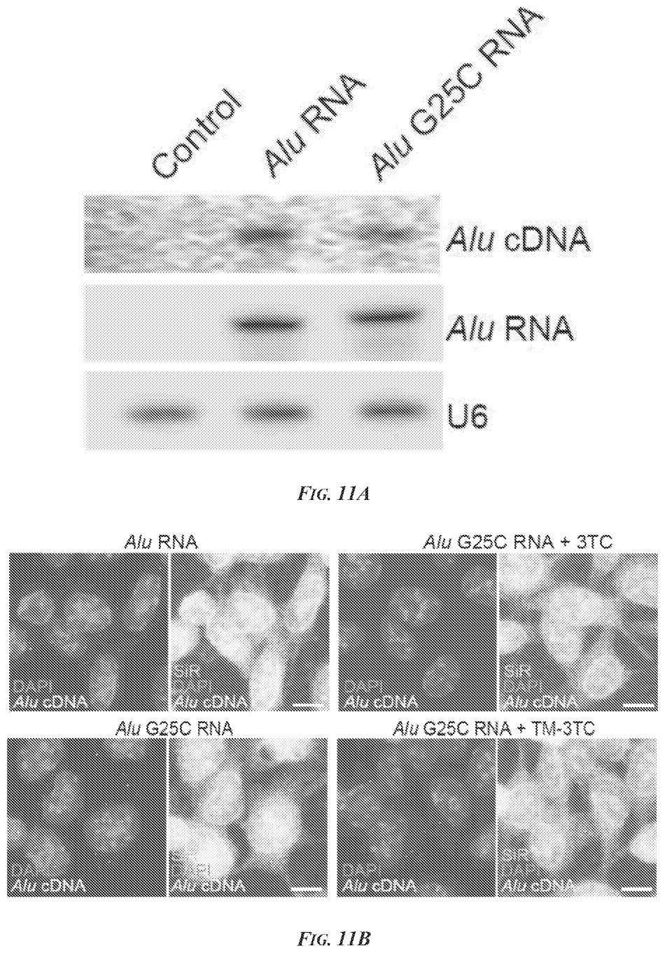

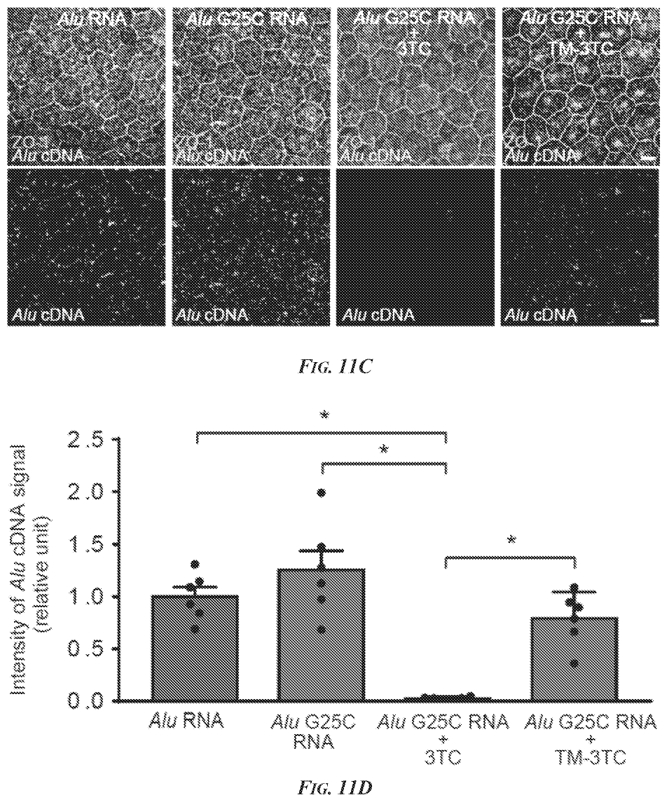

Next, we directly tested whether L1 was responsible for Alu cDNA formation. An L1 siRNA (FIGS. 10E and 10F) prevented the production of Alu cDNA in primary human RPE cells after Alu RNA transfection, heat shock, or DICER1 antisense treatment, as monitored by in situ hybridization (FIG. 7F) and by real-time PCR (FIG. 10G). Moreover, via in situ hybridization, we found that L1 siRNA reduced Alu cDNA formation in ARPE-19 cells after heat shock and DICER1 antisense treatments (FIG. 10H). Pharmacologic inhibition of L1 reverse transcriptase with high doses of the NNRTIs EFV and DLV34 also prevented Alu cDNA synthesis in primary human RPE cells after Alu RNA transfection, heat shock, or DICER1 knockdown (FIG. 7G). At high doses, EFV and DLV also prevented Alu cDNA formation in vivo after Alu RNA transfection in Casp1/4 dko mice (FIG. 10I). In contrast, NVP, which does not inhibit L1 reverse transcriptase, did not prevent Alu cDNA synthesis in primary human RPE cells (FIG. 7G) or after Alu RNA transfection in Casp1/4 dko mice (FIG. 10I). These findings are consistent with the concept that an overactive conversion of Alu RNA to Alu cDNA by L1 contributes to RPE cell death in human GA. Further supportive of the idea that Alu cDNA, but not retrotransposition, is essential for Alu RNA toxicity, we found that the retrotransposition-deficient Alu G25C mutant (FIGS. 2E-2G), which induced RPE degeneration (FIG. 2H) also induced Alu cDNA formation in mouse fibroblast cultures (FIGS. 11A and 11B) and in vivo in Casp1/4 dko mice (FIGS. 11C and 11D).

### Example 4

### Alu cDNA Mediates Alu RNA Toxicity in a Model of AMD

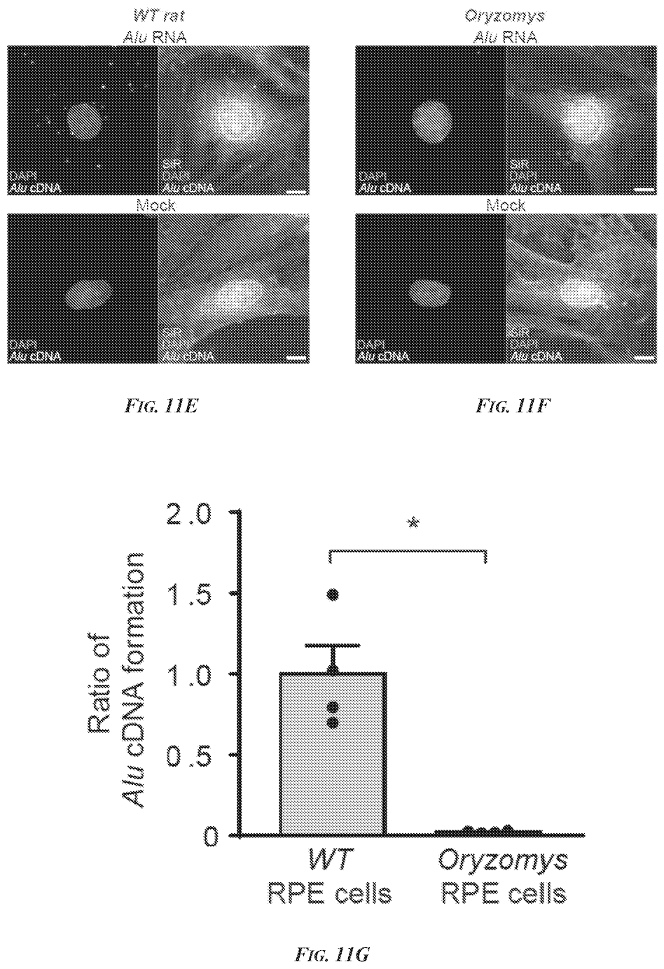

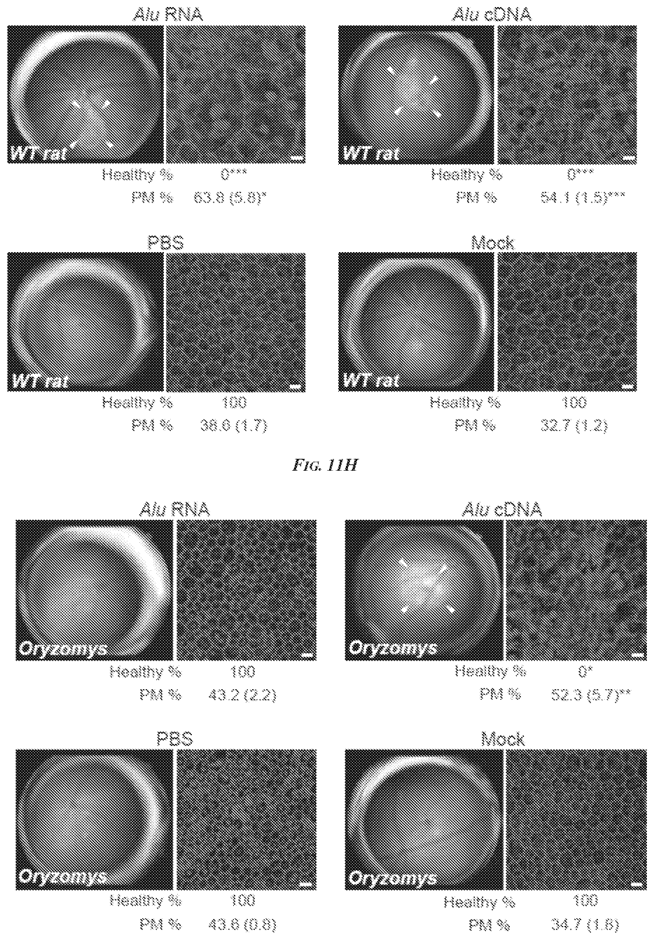

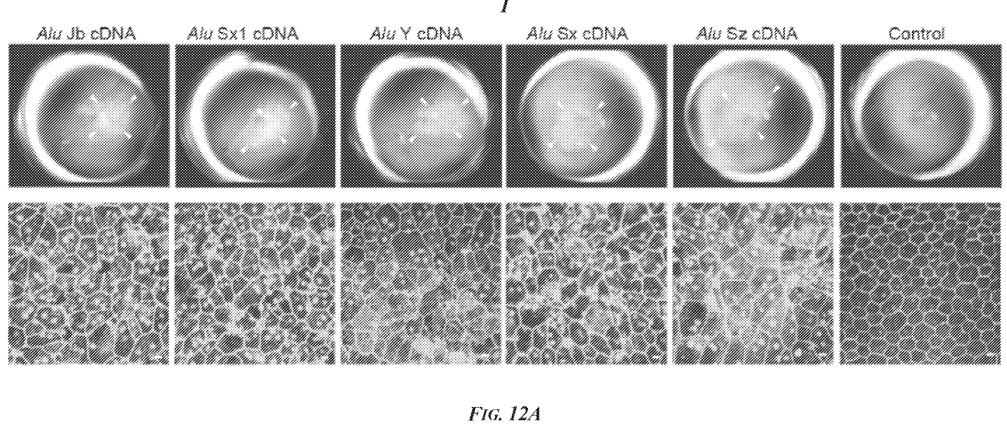

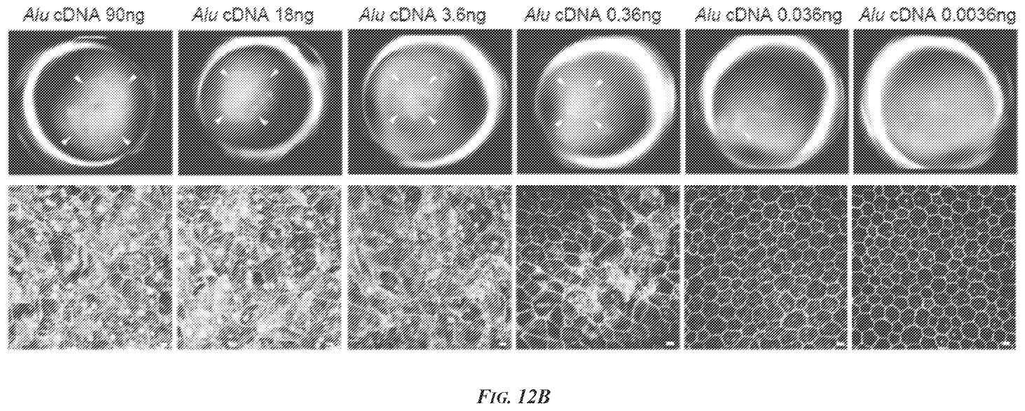

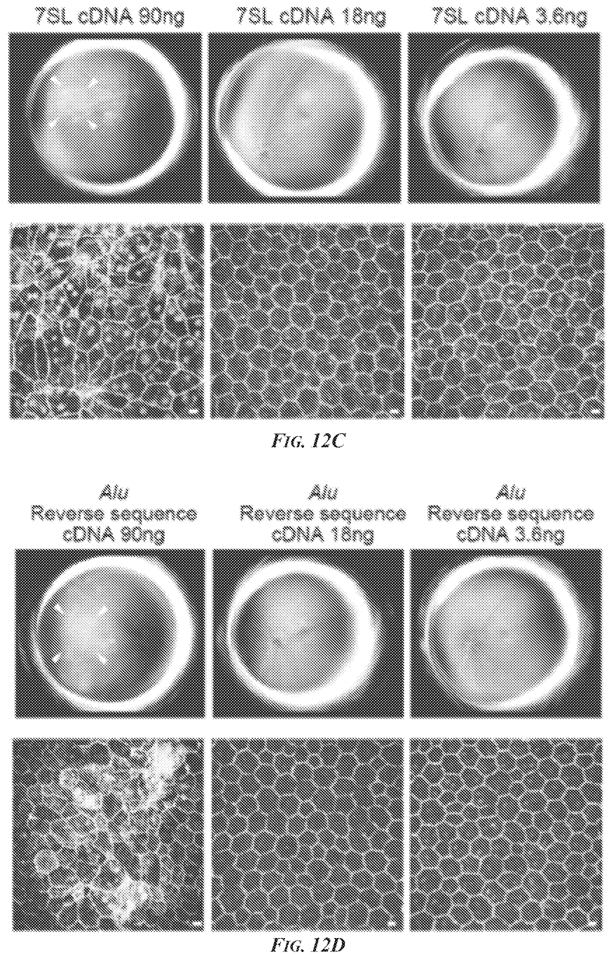

Next, we sought to determine whether Alu cDNA was cytotoxic in the absence of its RNA template. We synthesized single-stranded DNAs corresponding to the five most abundant Alu cDNA subfamily sequences identified; each cDNA induced RPE degeneration after subretinal injection in wild-type mice (FIG. 12A), suggesting that a particular subfamily of Alu is not essential for inducing retinal toxicity. Interestingly, on a molar basis, Alu cDNA (FIG. 12B) was at least 100-times more potent than Alu RNA (FIG. 2I) in inducing RPE death. Notably, a reverse sequence of Alu cDNA did not induce RPE degeneration, not did a DNA sequence complementary to 7SL RNA (FIGS. 12C and 12D). These data suggest a specificity to the RPE degeneration induced by Alu cDNA that seems to be influenced by its sequence and orientation.

To confirm that L1 activity and Alu cDNA formation were essential for Alu RNA toxicity, we performed experiments in rice rats (Oryzomys palustris) and in wild-type laboratory Brown-Norway rats (Rattus norvegicus). Unlike Rattus norvegicus, Oryzomys palustris is an “L1 extinct species” (Casavant et al., 2000; Grahn et al., 2005; Rinehart et al., 2005; Yang et al., 2014), i.e., it no longer has functionally mobile L1 elements due to acquisition of numerous insertions, deletions, and stop codons within formerly active L1 sequences. However, whether this rodent retains L1 reverse transcriptase activity is unknown. We found that Alu cDNA formation was dramatically reduced after Alu RNA transfection in Oryzomys palustris RPE cells compared with wild-type rat RPE cells (FIGS. 11E-11G), and that Alu RNA induced RPE degeneration in wildtype rats (FIG. 11H) but not in Oryzomys (FIG. 11I). In contrast, Alu cDNA induced RPE degeneration in both rat species (FIGS. 11H and 11I). Together, these data are consistent with the concept that L1-mediated formation of Alu cDNA is essential for Alu RNA toxicity.

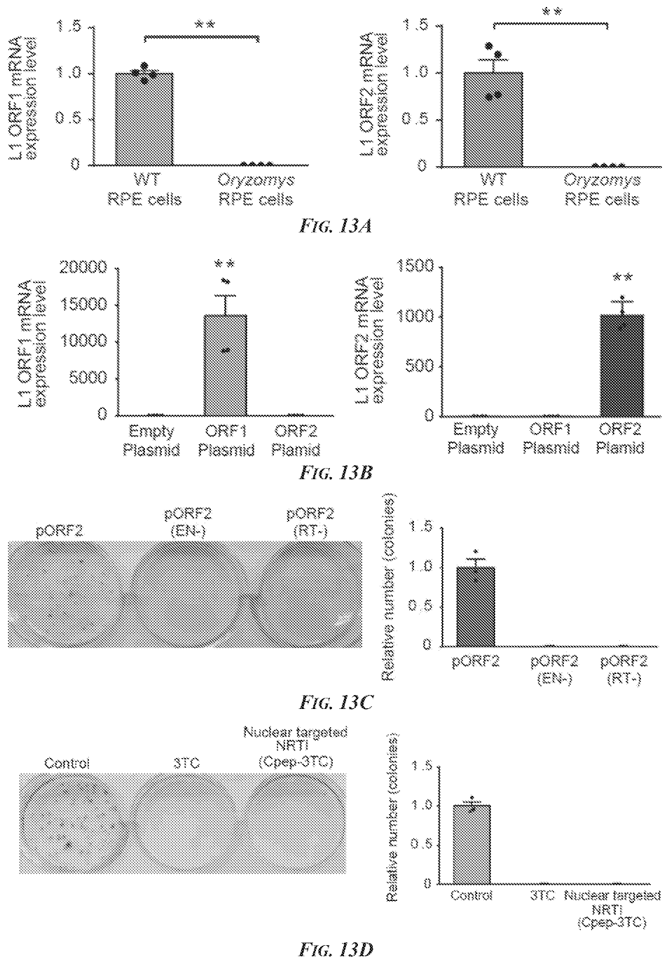

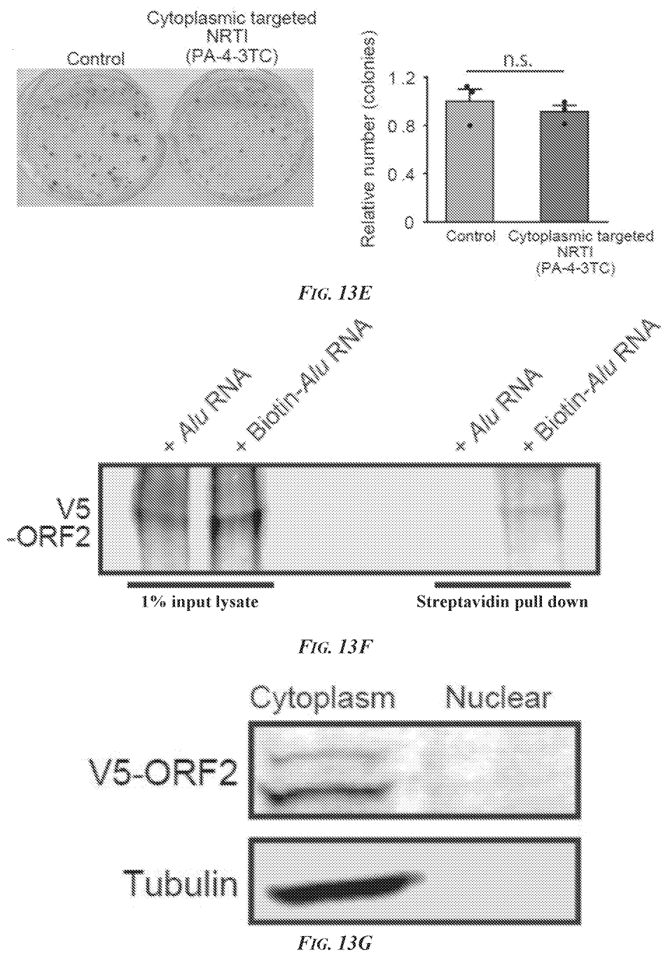

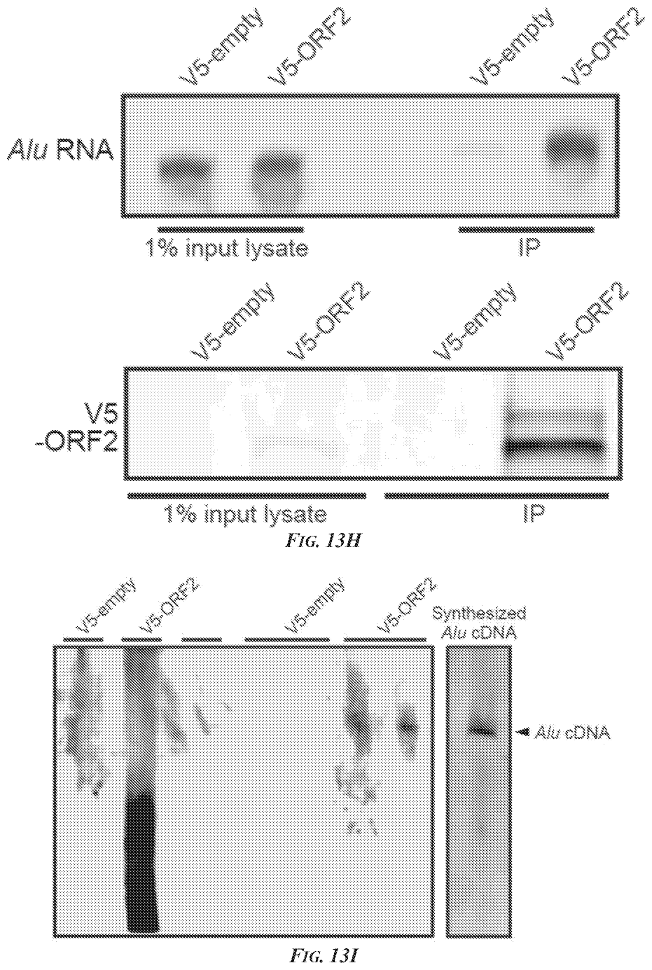

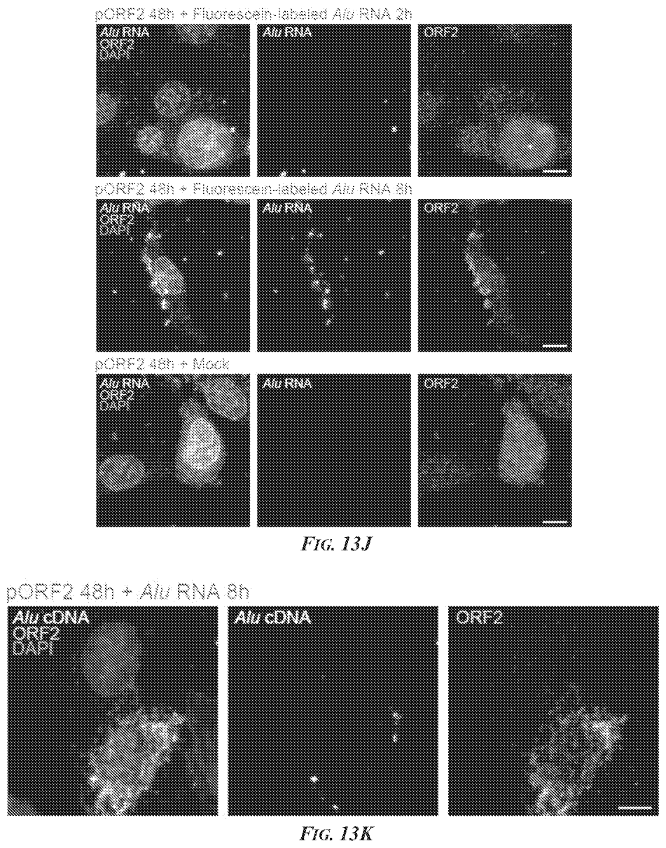

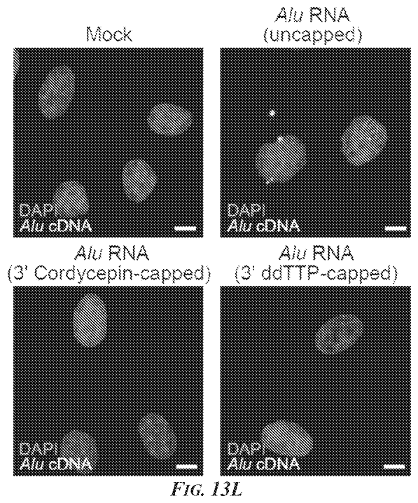

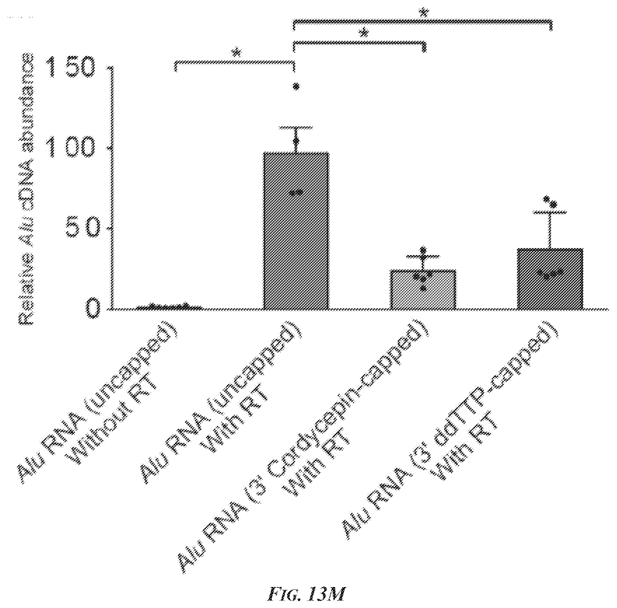

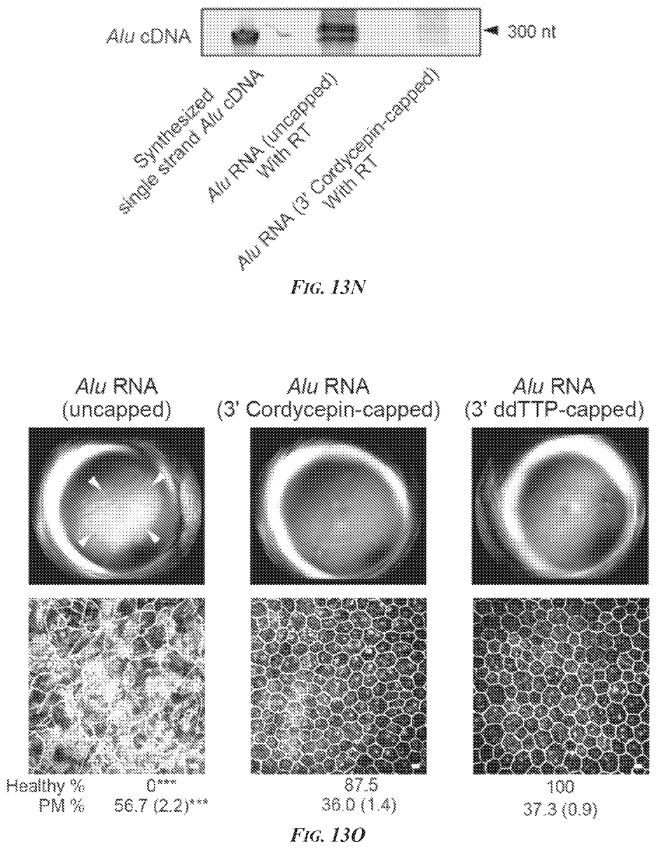

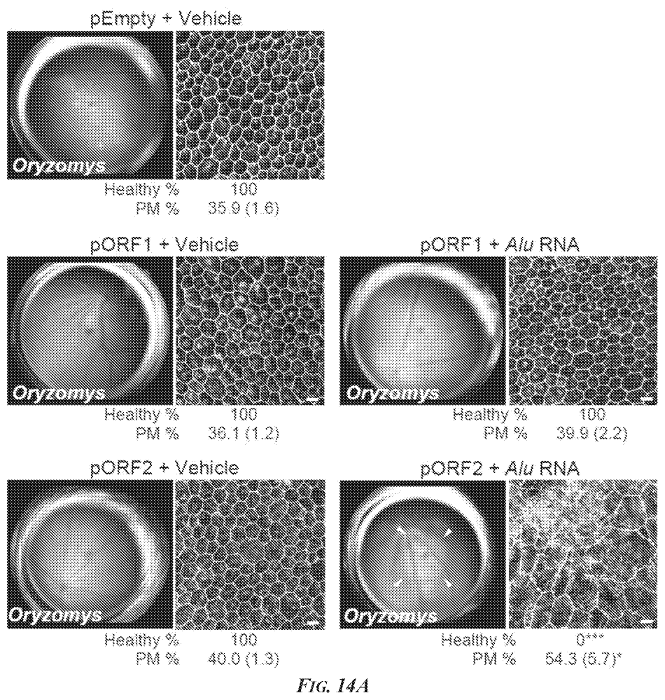

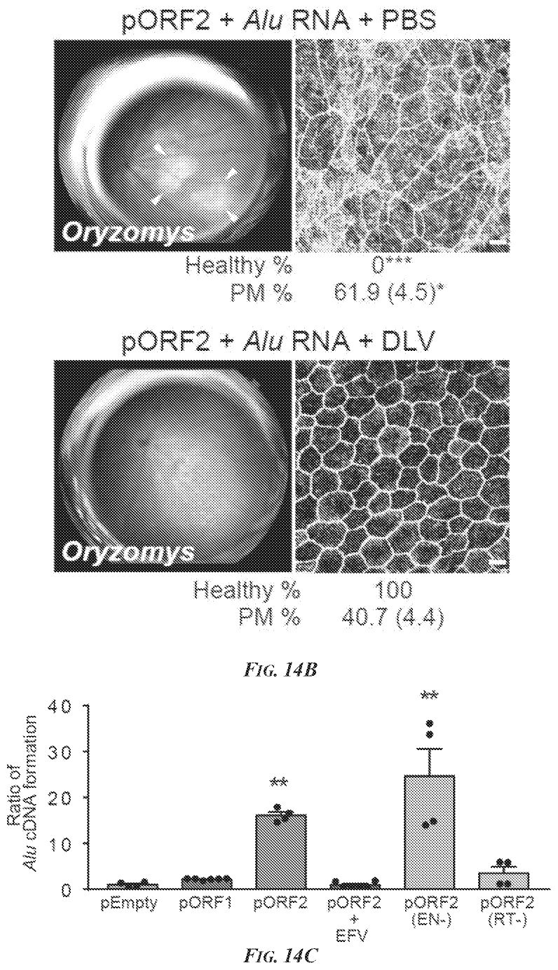

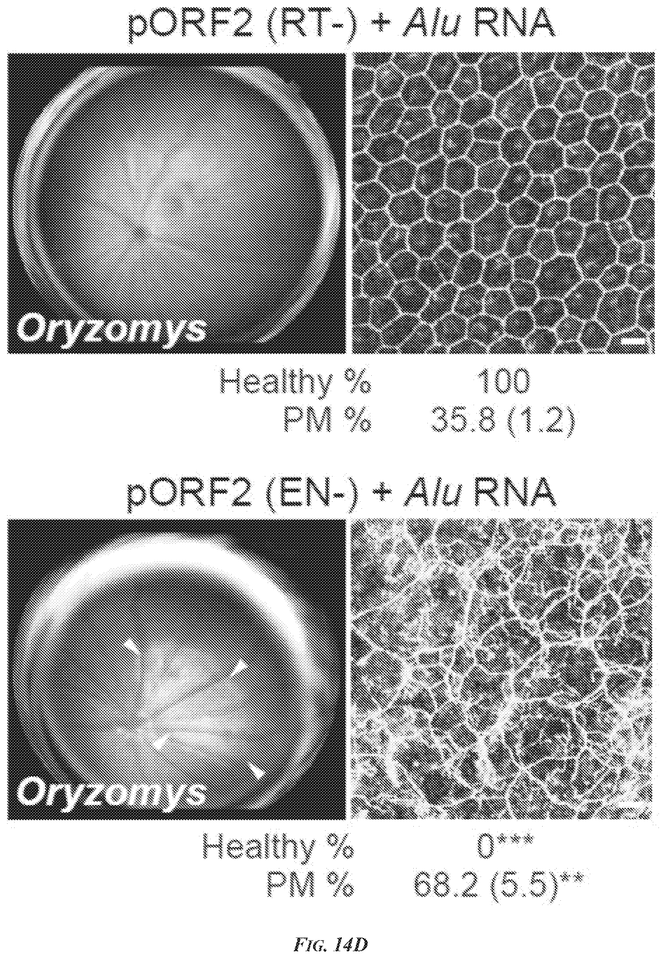

We confirmed the absence of L1 ORF1 and ORF2 RNAs in Oryzomys RPE cells (FIG. 13A), and the ability to enforce expression of ORF1 and ORF2 in these cells (FIG. 13B). We found that in vivo enforced expression of L1 ORF2p, but not of L1 ORF1p, in the RPE of Oryzomys, restored the ability of Alu RNA to induce RPE degeneration (FIG. 14A). Consistent with its ability to block L1 reverse transcriptase activity, high-dose DLV blocked this L1 ORF2p-facilitated Alu RNA-induced RPE degeneration in Oryzomys (FIG. 14B). Similarly, in Oryzomys RPE cells in culture, Alu RNA transfection induced the formation of Alu cDNA when expression of L1 ORF2p, but not of L1 ORF1p, was enforced; this was inhibited by high-dose EFV, consistent with its ability to block L1 reverse transcriptase activity (FIG. 14C).

Next, we enforced expression of an endonuclease-deficient (EN-) L1 ORF2p mutant (FIG. 13C) or a reverse transcriptase-deficient (RT-) L1 ORF2p mutant into Oryzomys RPE cells in culture. Alu RNA transfection induced the formation of Alu cDNA in the presence of L1 ORF2p (EN-) but not in the presence of L1 ORF2p (RT-) (FIG. 14C). Similarly, in vivo enforced expression of the (EN-) L1 ORF2p mutant restored the ability of Alu RNA to induce RPE degeneration in Oryzomys (FIG. 14D). In contrast, Alu RNA did not induce RPE degeneration in Oryzomys following in vivo enforced expression of the (RT-) L1 ORF2p mutant (FIG. 14D). These data identify L1 ORF2p's reverse transcriptase activity as crucial and endonuclease activity as dispensable for cytoplasmic Alu cDNA synthesis and Alu RNA-induced retinal toxicity.

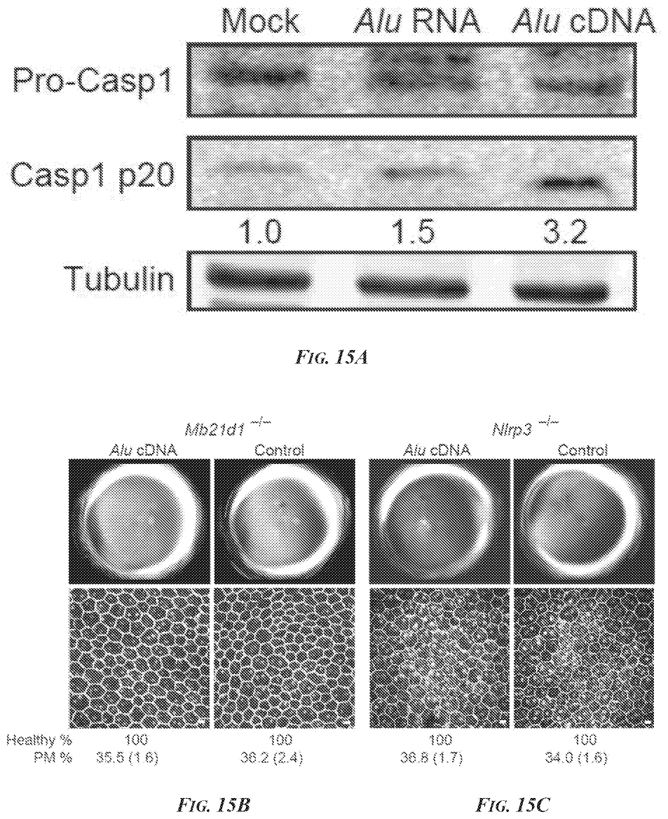

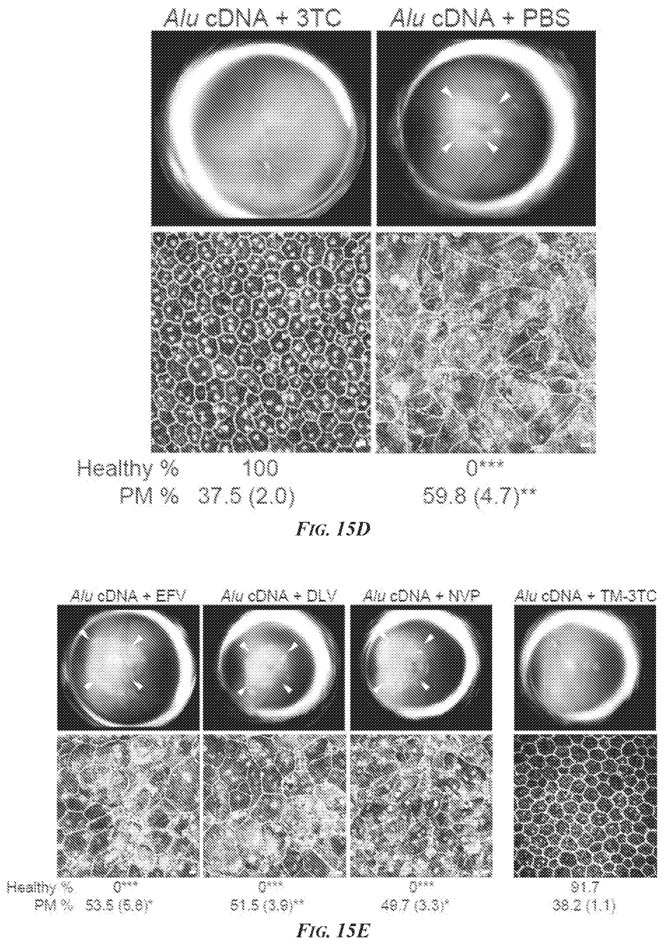

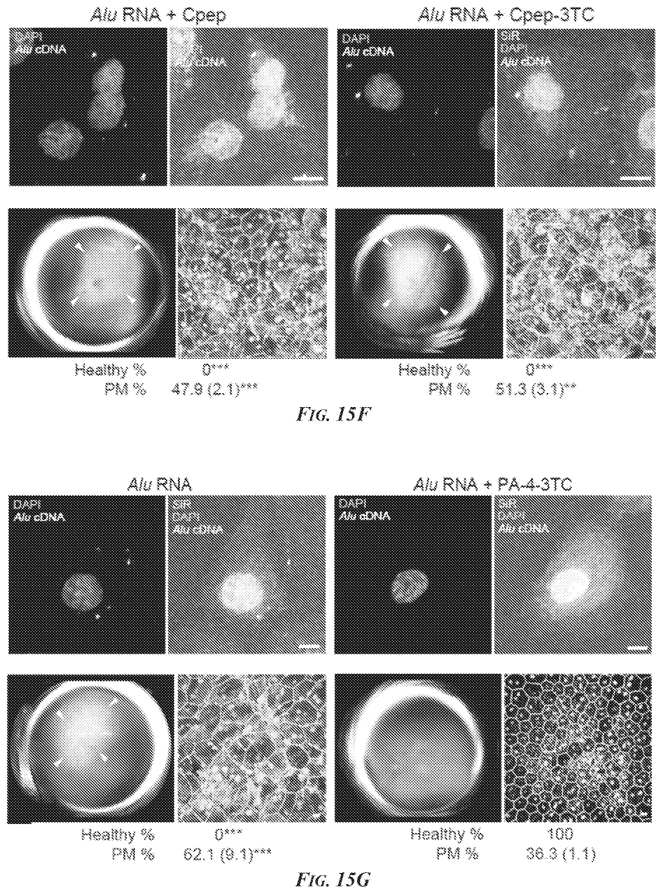

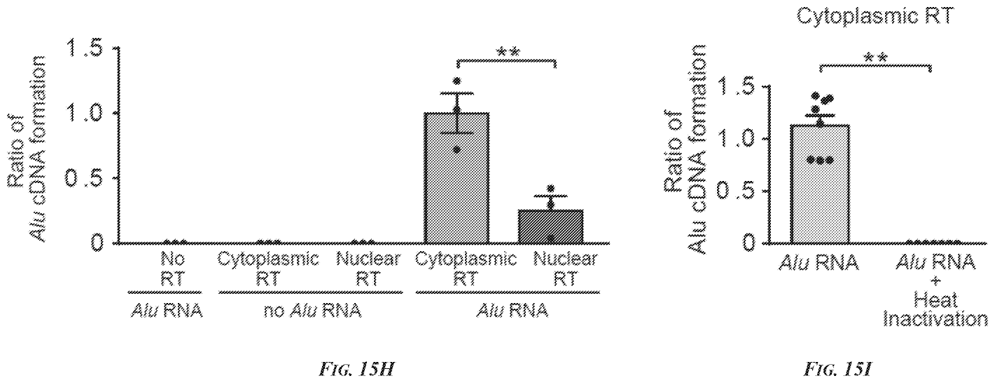

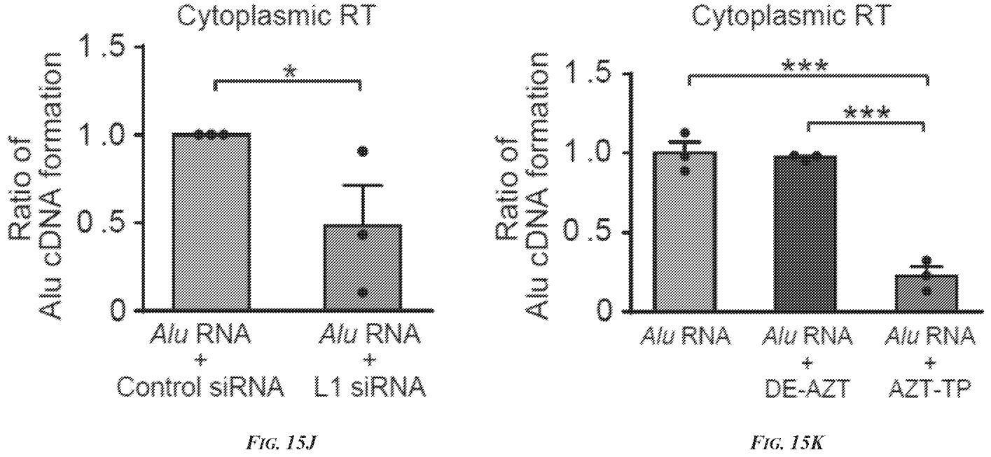

We next explored molecular pathways that mediate Alu cDNA toxicity. Recently, we identified cyclic GMP-AMP synthase (encoded by Mb21d1) signalling as a conduit of Alu RNA-induced NLRP3 inflammasome activation (Kerur et al., 2018). Therefore, we investigated whether these same inflammatory pathways were also involved in Alu cDNA toxicity. We found that Alu cDNA, like Alu RNA, activated caspase-1 in primary human RPE cells (FIG. 15A). Alu cDNA transfection led to greater caspase-1 activation compared to an equal quantity of Alu RNA, consistent with the in vivo observation that Alu cDNA was more potent than Alu RNA (FIGS. 2I and 12B). In addition, Alu cDNA did not induce RPE degeneration in Mb21d1^{-/-} or Nlrp3^{-/-} mice (FIGS. 15B and 15C).

Consistent with its intrinsic anti-inflammatory function9,50, 3TC protected against Alu cDNA toxicity (FIG. 15D). Therefore, 3TC appeared to have a dual protective effect against Alu RNA toxicity: blocking conversion of Alu RNA to Alu cDNA, and inhibiting Alu cDNA-mediated NLRP3 inflammasome signalling. On the other hand, L1 reverse transcriptase-inhibitory doses of NNRTIs did not block Alu cDNA toxicity, whereas TM-3TC, which blocks inflammasome activation, blocked RPE degeneration (FIG. 15E). Thus, in contrast to 3TC, the protective effect of high dose NNRTIs against Alu RNA toxicity is due solely to their inhibition of reverse transcriptase, whereas their inability to protect against Alu cDNA toxicity is due to their lack of NLRP3 inflammasome antagonism. Collectively, these data indicate that Alu cDNA is mechanistically interposed between Alu RNA and inflammasome activation, and that inflammation inhibition is sufficient to block RPE degeneration induced by Alu RNA or Alu cDNA.

### Example 5

### Cytoplasmic Synthesis of Alu cDNA

Canonically, reverse transcription of Alu RNA by L1 is thought to occur in the nucleus (Wallace et al., 2008; Kroutter et al., 2009; Wagstaff et al., 2012), a process that is coupled with integration of Alu DNA into the genome. However, it is not known whether Alu cDNA also is synthesized in the cytoplasm. To determine the locus of Alu cDNA synthesis, we used formulations of 3TC that restrict it to the nuclear or cytoplasmic compartments. Conjugation of 3TC with a cyclic peptide (Cpep) targets this compound for nuclear localization (Mandal et al., 2011), whereas a mixture of an amino acid/fatty acyl moiety (PA-4) restricts 3TC to the cytoplasmic compartment (Nasrolahi Shirazi et al., 2013). Consistent with the fact that reverse transcription of Alu occurs in the nucleus during retrotransposition, we confirmed that Cpep-3TC, but not PA-4-3TC, blocked Alu retrotransposition (FIGS. 13D and 13E). In contrast, cytoplasmic Alu cDNA formation following Alu RNA transfection in primary human RPE cells and Alu RNA-induced RPE degeneration in wild-type mice were blocked by PA-4-3TC but not Cpep-3TC (FIGS. 15F and 15G), indicating that inhibition of cytoplasmic reverse transcriptase activity is critical for preventing Alu RNA toxicity and that this toxic Alu cDNA did not leak from the nucleus to the cytoplasm following aborted retrotransposition.

We then performed an ex vivo reverse transcription assay of Alu cDNA synthesis by incubating protein extracts of nuclear or cytoplasmic fractions of a mouse embryonal carcinoma cell line (F9), which is high in L1 expression (Martin, 1991), or of wild-type mouse RPE cells with Alu RNA. We observed higher amounts of Alu cDNA formation with cytoplasmic fractions than with nuclear fractions in both cell types (FIG. 15H). Heat denaturation of cytoplasmic fractions eliminated Alu cDNA synthesis (FIG. 15I), compatible with its formation by a protein enzyme. We also found that treatment of cells with L1 siRNA resulted in reduced Alu cDNA production in the cytoplasmic fractions compared with control siRNA treatment (FIG. 15J). In addition, treatment of the cytoplasmic extracts with AZT-triphosphate (AZT-TP), the active form of the NRTI AZT that inhibits reverse transcription, reduced Alu cDNA synthesis, whereas diethyl-AZT (DE-AZT), which does not block reverse transcriptase (Fowler et al., 2014; Ambati et al., 2018), did not inhibit Alu cDNA formation (FIG. 15K). These data support the conclusion that Alu cDNA can be synthesized via L1-mediated reverse transcription in the cytoplasm, and that this cytoplasmic Alu cDNA is responsible for its retinal cytotoxicity.

To monitor Alu cDNA formation in the cytoplasm, we probed the molecular association between L1 ORF2p, Alu RNA, and Alu cDNA in RNase H2-deficient HeLa cells co-expressing V5-tagged L1 ORF2p and biotinylated-Alu RNA. Using pull-down assays, we captured the association of Alu RNA with L1 ORF2p in cytoplasmic extracts of these cells, as well as that of Alu cDNA with L1 ORF2p (FIGS. 13F-13I). We then transfected Oryzomys RPE cells with pORF2 and fluorescent-labelled Alu RNA, and colocalized both Alu RNA and Alu cDNA with ORF2 (FIGS. 13I and 13K). These data support a model in which Alu RNA associated with L1 ORF2p in the cytoplasm and is reverse transcribed into Alu cDNA.

In the canonical model of Alu retrotransposition, reverse transcription of Alu RNA by L1 in the nucleus occurs via target-primed reverse transcription, wherein the endonuclease activity of L1 exposes an oligo-T stretch of genomic DNA that serves to prime reverse transcription of the 3′ oligo-A stretch of Alu RNA (Feng et al., 1996). We sought to determine how priming of Alu reverse transcription might occur in the cytoplasm. Alu RNA is capable of intramolecular base-pairing (Ahl et al., 2015); therefore, we hypothesized that Alu could be capable of self-priming using the 3′ U-stretch. Indeed, there is precedent for a repetitive RNA—rodent BC1 RNA—priming its own reverse transcription (Shen et al., 1997). To test this, we disabled the self-priming capability of an in vitro synthesized Alu RNA via 3′ capping with the chain terminators 2′,3′-dideoxythymidine-5′-triphosphate (ddTTP) or cordycepin (3′-deoxyadenosine). Consistent with this hypothesis, transfection of these 3′ capped Alu RNA species into wild-type mouse RPE cells stunted Alu cDNA formation (FIG. 13L). Also supportive, in an in-tube reverse transcriptase assay, incubating wild-type mouse RPE cell cytoplasmic protein extracts with uncapped Alu RNA resulted in far more Alu cDNA synthesis than with 3′ capped Alu RNA (FIGS. 13M and 13N). As in vivo confirmation of this concept, we found that subretinal administration of 3′ capped Alu RNA species did not induce RPE degeneration in wild-type mice (FIG. 13O). These data suggest that self-priming could be one mechanism by which Alu reverse transcription occurs in the cytoplasm.

### Example 6

### NRTIs are Associated with Lower Risk of Atrophic AMD

Given the experimental and human tissue data that implicate Alu cDNA in GA pathogenesis, and the protective role of NRTIs in the Alu-induced model of RPE degeneration, we assessed whether NRTI use is associated with altered risk of atrophic AMD in humans. We analysed the effects of NRTIs in two longitudinal analyses of incident atrophic AMD among HIV-positive persons in the United States: the U.S. Veterans Health Administration database, which contains electronic medical records of approximately 10 million former members of the U.S. Armed Forces over the time period 2000-2017, and the PearlDiver database, which contains approximately 20 million patient records obtained from analysis of commercial insurance claims from Humana health insurance beneficiaries over the time period 2007-2016. We tested the NRTI class of drugs as a time-dependent covariate in a Cox proportional hazard model. We adjusted for age, sex, ethnicity, use of NNRTIs or protease inhibitors, body mass index, tobacco use, CD4⁺ cell count, HIV viral load, and Charlson Comorbidity Index score. Exposure to NRTIs was associated with a reduced risk of a new diagnosis of atrophic AMD (Table 2) in both the Veterans (adjusted hazard ratio=0.765, 95% CI, 0.588, 0.996; n=25,923) and PearlDiver databases (adjusted hazard ratio=0.442, 95% CI, 0.252, 0.779; n=13,322).

TABLE 2
Incident Dry Age-related Macular Degeneration
Among HIV-positive Persons

| | | | Hazard Ratio (95% CI) | |
|---|---|---|---|---|
| | Users | Cases | Unadjusted | Adjusted |
| Veterans | | | | |
| Never User | 9,079 | 306 | 1 | 1 |
| Ever User | 16,844 | 393 | 0.430 | 0.765 |
| | | | (0.367, 0.502) | (0.588, 0.996) |
| Humana | | | | |
| Never User | 2,008 | 47 | 1 | 1 |
| Ever User | 11,314 | 66 | 0.364 | 0.442 |
| | | | (0.244, 0.543) | (0.252, 0.779) |

Incident atrophic age-related macular degeneration rates were less frequent among HIV-positive persons exposed to nucleoside reverse transcriptase inhibitors (NRTIs; Ever User) compared with those never exposed to NRTIs (Never User). U.S. Veterans Health Administration (VHA; 2000-2017; Veterans) and PearlDiver Humana (PD; 2007-2016; Humana) databases. CI, confidence interval; ref, reference group. Hazard Ratios, estimated by Cox proportional hazards regression, adjusted for age, sex, ethnicity, use of NNRTIs or protease inhibitors, body mass index, tobacco use, CD4⁺ cell count, HIV viral load, and Charlson Comorbidity Index score.

### Discussion of Examples

The principal hazard of L1 to the human genome is perceived to be mutagenic retrotransposition. However, our findings expand this threat assessment to reverse transcription as well, thus revealing a more insidious mechanism of human disease driven by reverse transcription of host genetic material. The low levels of Alu cDNA in the non-diseased states suggest the possibility of cellular systems to combat reverse transcription. Given the evolutionary arms race between retrotransposons and cytoprotective pathways, genes undergoing rapid adaptive evolution are likely candidates to be such centurions.

Previously, we demonstrated that NRTIs block Alu RNA-induced RPE degeneration by virtue of inhibiting inflammasome activation (Fowler et al., 2014). Our work redefines the protection conferred by NRTIs against Alu toxicity as being derived from their inhibition of both reverse transcriptase and inflammasome activation. Our composite data from disease modelling, tissue sampling, and population database analyses provide a rationale for prospective testing of NRTIs or alkylated NRTI derivatives, which are less toxic than NRTIs (Fowler et al., 2014), to treat GA. Our findings also proffer Alu cDNA, which is enriched in the junctional zone interposed between atrophic and healthy regions of the retina, as a pathogenic candidate for the centrifugal expansion of GA, whose expansion characteristics have defied explanation.

Recently, it has been proposed that L1 promotes pathology in a cell culture model of Aicardi-Goutières syndrome via the accumulation of immune-activating L1 reverse transcripts (Thomas et al., 2017). However, it was not clear whether L1 cytoplasmic single-stranded DNAs were bona fide reverse transcripts formed in the cytoplasm or, alternatively, whether those L1 DNAs represent aborted retrotransposition-fragments that subsequently enter the cytoplasm (Dewannieux et al., 2003; Yang et al., 2007; Stetson et al., 2008; Wallace et al., 2008; Kroutter et al., 2009; Reijns et al., 2012; Wagstaff et al., 2012; Pokatayev et al., 2016). The presently disclosed subject matter fills several important knowledge gaps in this nascent field. For one, we provide direct evidence that endogenous Alu cDNAs are full-length reverse transcripts that are synthesized in the cytoplasm by L1, and that these single-stranded cDNAs are not products of aborted retrotransposition. We also found that endogenous Alu cDNAs are produced, are toxic in vivo, and are detectable in excess from diseased tissue of patients with AMD. The apparent clustering of some of these sequences near polymorphic loci statistically associated with AMD is of unclear biological significance, which could be explored in future investigations.

Another potential source of reverse transcriptase activity is the human endogenous retrovirus (HERV) family of retrotransposons. Although HERVs are considered to be immobile in the genome (Beck et al., 2011; Weiss, 2016), elevated mRNA levels have been reported in some human diseases (Li et al., 2015; Mager et al., 2015). Multiple lines of evidence suggest that HERV reverse transcriptase activity is not responsible for Alu cDNA production or RPE degeneration. Alu RNA-induced Alu cDNA synthesis and retinal toxicity were inhibited by L1 knockdown but not by NVP, which inhibits HERV reverse transcriptase (Tyagi et al., 2017). Furthermore, endogenous retroviruses have undergone an expansion in Oryzomys palustris (Erickson et al., 2011), yet Alu RNA administration did not induce Alu cDNA production or RPE degeneration in this L1-inactive rodent.

Our findings also raise questions regarding mechanistic details of L1 function. In the canonical retrotransposition pathway, L1 protein binds to substrate RNA in the cytoplasm; the L1-RNA complex is shuttled to the nucleus where it is reverse transcribed and integrated into a chromosome. In contrast, our data indicate that L1-mediated reverse transcription of substrate RNAs also occurs in the cytoplasm, and that inhibition of cytoplasmic reverse transcriptase prevents Alu RNA toxicity. In addition, a conglomerate of L1-interacting proteins in the L1 ribonucleoprotein particle is known to regulate retrotransposition (Goodier et al., 2013); it would be interesting to determine the effect of the L1 interactome on endogenous cDNA formation. Moreover, L1 has clear substrate specificity for retrotransposing AluY sequences, whereas we found that AluS subfamily sequences are the predominant endogenous Alu cDNA in RPE cells, suggesting a potential dichotomy in substrate preference in nuclear versus cytoplasmic L1 reverse transcriptase.

More broadly, it will be interesting to address the possibility that a variety of host cytoplasmic RNAs could be templates for endogenous cDNA formation. Previous work has found that mRNAs can serve as L1 substrates for retrotransposition, albeit at lower efficiency than Alu or L1 substrates (Wei et al., 2001; Dewannieux et al., 2003); future studies are needed to determine the relative efficiency of retroelements versus other RNAs in endogenous cDNA formation (Dhellin et al., 1997; Esnault et al., 2000). As L1 also drives speciation and enhances interand intra-individual genetic diversity (Kazazian et al., 2017), the presently disclosed subject matter raises the intriguing question of whether L1-derived cDNAs modulate the evolutionary impact of L1. We also speculate that this proposed class of novel endogenous DNAs could mediate selectively advantageous physiologic functions ranging from immunogenicity, endogenous antisense gene regulation, and guiding proteins to specific nucleic acid targets.

It is also clear that disease-causing retroviruses such as HIV-1 reverse transcribe host messenger RNAs (Pulsinelli & Temin, 1991; Dunn et al., 1992), although the relevance of host-derived endogenous cDNA in human disease has not been well defined until now. Future work should determine the contribution of host endogenous cDNA in the pathogenesis of retroviral infections.

### REFERENCES

All references listed in the instant disclosure, including but not limited to all patents, patent applications and publications thereof, scientific journal articles, and database entries (including but not limited to GENBANK® biosequence database entries and all citation and annotations presented therein) are incorporated herein by reference in their entireties to the extent that they supplement, explain, provide a background for, and/or teach methodology, techniques, and/or compositions employed herein.
Ach et al. (2015) Lipofuscin redistribution and loss accompanied by cytoskeletal stress in retinal pigment epithelium of eyes with age-related macular degeneration. Invest Ophthalmol Vis Sci 56:3242-3252.
Ahl et al. (2015) Retrotransposition and Crystal Structure of an Alu RNP in the Ribosome-Stalling Conformation. Mol Cell 60:715-727.
Ambati et al. (2003) Age-related macular degeneration: etiology, pathogenesis, and therapeutic strategies. Sury Ophthalmol 48:257-293.
Baltimore (1970) RNA-dependent DNA polymerase in virions of RNA tumour viruses. Nature 226:1209-1211.
Banuelos-Sanchez et al. (2019) Synthesis and Characterization of Specific Reverse Transcriptase Inhibitors for Mammalian LINE-1 Retrotransposons. Cell Chemical Biology 26:1095-1109.
Batzer & Deininger (2002) Alu repeats and human genomic diversity. Nat Rev Genet 3:370-379.
Beck et al. (2011) LINE-1 elements in structural variation and disease. Annu Rev Genomics Hum Genet 12:187-215.
Bennett et al. (2008) Active Alu retrotransposons in the human genome. Genome Res 18:1875-1883.
Bonilha et al. (2011) Histopathology and functional correlations in a patient with a mutation in RPE65, the gene for retinol isomerase. Invest Ophthalmol Vis Sci 52:8381-8392.
Bonilha et al. (2015a) Histopathological comparison of eyes from patients with autosomal recessive retinitis pigmentosa caused by novel EYS mutations. Graefes Arch Clin Exp Ophthalmol 253:295-305.
Bonilha et al. (2015b) Histopathology of the Retina from a Three Year-Old Suspected to Have Joubert Syndrome. Austin J Clin Ophthalmol 2:1057.
Bonilha et al. (2016) Retinal Histopathology in Eyes from a Patient with Stargardt disease caused by Compound Heterozygous ABCA4 Mutations. Ophthalmic Genet 37:150-160.
Booij et al. (2010) A new strategy to identify and annotate human RPE-specific gene expression. PLoS One 5:e9341.
Casavant et al. (2000) The end of the LINE?: lack of recent L1 activity in a group of South American rodents. Genetics 154:1809-1817.
Chang et al. (1997) A highly conserved nucleotide in the Alu domain of SRP RNA mediates translation arrest through high affinity binding to SRP9/14. Nucleic Acids Res 25:1117-1122.
Chun et al. (1997) Quantification of latent tissue reservoirs and total body viral load in HIV-1 infection. Nature 387:183-188.
Crick (1970) Central dogma of molecular biology. Nature 227:561-563.
D'Haene et al. (2010) Accurate and objective copy number profiling using real-time quantitative PCR. Methods 50:262-270.
Dai et al. (2011) Effect of reverse transcriptase inhibitors on LINE-1 and Ty 1 reverse transcriptase activities and on LINE-1 retrotransposition. BMC Biochem 12:18.
De Luca et al. (2016) Enhanced expression of LINE-1-encoded ORF2 protein in early stages of colon and prostate transformation. Oncotarget 7:4048-4061.
Deininger & Batzer (2002) Mammalian retroelements. Genome Res 12:1455-1465.
Deininger (2011) Alu elements: know the SINEs. Genome Biol 12:236.
Dewannieux et al. (2003) LINE-mediated retrotransposition of marked Alu sequences. Nat Genet 35:41-48.
Dhellin et al. (1997) Functional differences between the human LINE retrotransposon and retroviral reverse transcriptases for in vivo mRNA reverse transcription. EMBO J 16:6590-6602.
Dobin et al. (2013) STAR: ultrafast universal RNA-seq aligner. Bioinformatics 29:15-21.
Dridi et al. (2012) ERK1/2 activation is a therapeutic target in age-related macular degeneration. Proc Natl Acad Sci USA 109:13781-13786.
Dunn et al. (1992) Characterization of unintegrated retroviral DNA with long terminal repeat-associated cell-derived inserts. J Virol 66:5735-5743.
Erickson et al. (2011) Retrofitting the genome: L1 extinction follows endogenous retroviral expansion in a group of muroid rodents. J Virol 85:12315-12323.
Esnault et al. (2000) Human LINE retrotransposons generate processed pseudogenes. Nat Genet 24:363-367.
Feng et al. (1996) Human L1 retrotransposon encodes a conserved endonuclease required for retrotransposition. Cell 87:905-916.
Fowler et al. (2014) Nucleoside reverse transcriptase inhibitors possess intrinsic antiinflammatory activity. Science 346:1000-1003.
Fritsche et al. (2016) A large genome-wide association study of age-related macular degeneration highlights contributions of rare and common variants. Nat Genet 48:134-143.
Goodier et al. (2013) Mapping the LINE1 ORF1 protein interactome reveals associated inhibitors of human retrotransposition. Nucleic Acids Res 41:7401-7419.
Grahn et al. (2005) Extinction of LINE-1 activity coincident with a major mammalian radiation in rodents. Cytogenet Genome Res 110:407-415.
Grossniklaus et al. (2013) Anatomic alterations in aging and age-related diseases of the eye. Invest Ophthalmol Vis Sci 54:ORSF23-27.
Hariri et al. (2016) Retinal Sensitivity at the Junctional Zone of Eyes With Geographic Atrophy Due to Age-Related Macular Degeneration. Am J Ophthalmol 168:122-128.
Hoebeeck et al. (2005) Rapid detection of VHL exon deletions using real-time quantitative PCR. Lab Invest 85:24-33.
Holz et al. (2001) Fundus autofluorescence and development of geographic atrophy in age-related macular degeneration. Invest Ophthalmol Vis Sci 42:1051-1056.
Hung et al. (2015) The Ro60 autoantigen binds endogenous retroelements and regulates inflammatory gene expression. Science 350:455-459.
Italiani et al. (2014) Evaluating the levels of interleukin-1 family cytokines in sporadic amyotrophic lateral sclerosis. J Neuroinflammation 11:94.
Jelinek et al. (1980) Ubiquitous, interspersed repeated sequences in mammalian genomes. Proc Natl Acad Sci USA 77:1398-1402.
Johann et al. (2015) NLRP3 inflammasome is expressed by astrocytes in the SOD1 mouse model of ALS and in human sporadic ALS patients. Glia 63:2260-2273.
Jurka & Smith (1988) A fundamental division in the Alu family of repeated sequences. Proc Natl Acad Sci USA 85:4775-4778.
Kahlenberg et al. (2011) Inflammasome activation of IL-18 results in endothelial progenitor cell dysfunction in systemic lupus erythematosus. J Immunol 187:6143-6156.
Kaneko et al. (2011) DICER1 deficit induces Alu RNA toxicity in age-related macular degeneration. Nature 471:325-330.
Kazazian & Moran (2017) Mobile DNA in Health and Disease. N Engl J Med 377:361-370.
Kerur et al. (2013) TLR-independent and P2X7-dependent signaling mediate Alu RNA-induced NLRP3 inflammasome activation in geographic atrophy. Invest Ophthalmol Vis Sci 54:7395-7401.
Kerur et al. (2018) cGAS drives noncanonical-inflammasome activation in age-related macular degeneration. Nat Med 24:50-61.
Kim et al. (2014) DICER1/Alu RNA dysmetabolism induces Caspase-8-mediated cell death in age-related macular degeneration. Proc Natl Acad Sci USA 111:16082-16087.
Kirilyuk et al. (2008) Functional endogenous LINE-1 retrotransposons are expressed and mobilized in rat chloroleukemia cells. Nucleic Acids Res 36:648-665.
Kleinman et al. (2008) Sequence- and target-independent angiogenesis suppression by siRNA via TLR3. Nature 452:591-597.
Kleinman et al. (2012) Short-interfering RNAs induce retinal degeneration via TLR3 and IRF3. Mol Ther 20:101-108.
Krolewski et al. (1984) Structure of extrachromosomal circular DNAs containing both the Alu family of dispersed repetitive sequences and other regions of chromosomal DNA. J Mol Biol 174:41-54.
Kroutter et al. (2009) The RNA polymerase dictates ORF1 requirement and timing of LINE and SINE retrotransposition. PLoS Genet 5e1000458.
Lander et al. (2001) Initial sequencing and analysis of the human genome. Nature 409:860-921.
Li et al. (2015) Human endogenous retrovirus-K contributes to motor neuron disease. Sci Transl Med 7:307ra153.
Liao et al. (2014) featureCounts: an efficient general purpose program for assigning sequence reads to genomic features. Bioinformatics 30:923-930.
Liu et al. (1995) Cell stress and translational inhibitors transiently increase the abundance of mammalian SINE transcripts. Nucleic Acids Res 23:1758-1765.
Mackenzie et al. (2016) Ribonuclease H2 mutations induce a cGAS/STING-dependent innate immune response. EMBO J 35:831-844.
Mager & Stoye (2015) Mammalian Endogenous Retroviruses. Microbiol Spectr 3:MDNA3-0009-2014.
Mandal et al. (2011) Cell-penetrating homochiral cyclic peptides as nuclear-targeting molecular transporters. Angew Chem Int Ed Engl 50:9633-9637.
Martin (1991) Ribonucleoprotein particles with LINE-1 RNA in mouse embryonal carcinoma cells. Mol Cell Biol 11:4804-4807.
Masters et al. (2011) The inflammasome in atherosclerosis and type 2 diabetes. Sci Transl Med 3:81p517.
Mathias et al. (1991). Reverse transcriptase encoded by a human transposable element. Science 254:1808-1810.
Merluzzi et al. (1990) Inhibition of HIV-1 replication by a nonnucleoside reverse transcriptase inhibitor. Science 250:1411-1413.
Mills et al. (2007) Which transposable elements are active in the human genome? Trends Genet 23:183-191.
Mizutani et al. (2015) Nucleoside Reverse Transcriptase Inhibitors Suppress Laser-Induced Choroidal Neovascularization in Mice. Invest Ophthalmol Vis Sci 56:7122-7129.
Moran et al. (1996) High frequency retrotransposition in cultured mammalian cells. Cell 87:917-927.
Nasrolahi et al. (2013) Peptide amphiphile containing arginine and fatty acyl chains as molecular transporters. Mol Pharm 10:4717-4727.
Ostertag et al. (2000) Determination of L1 retrotransposition kinetics in cultured cells. Nucleic Acids Res 28:1418-1423.
PCT International Patent Application Publication Nos. WO 1988/04300; WO 2004/045543; WO 2004/090105; WO 2005/078094; WO 2018/136920.
Pokatayev et al. (2016) RNase H2 catalytic core Aicardi-Goutieres syndrome-related mutant invokes cGAS-STING innate immune-sensing pathway in mice. J Exp Med 213:329-336.
Prudencio et al. (2017) Repetitive element transcripts are elevated in the brain of C9orf72 ALS/FTLD patients. Hum Mol Genet 26:3421-3431.
Pulsinelli & Temin (1991) Characterization of large deletions occurring during a single round of retrovirus vector replication: novel deletion mechanism involving errors in strand transfer. J Virol 65:4786-4797.
Reijns et al. (2012) Enzymatic removal of ribonucleotides from DNA is essential for mammalian genome integrity and development. Cell 149:1008-1022.
Rinehart et al. (2005) SINE extinction preceded LINE extinction in sigmodontine rodents: implications for retrotranspositional dynamics and mechanisms. Cytogenet Genome Res 110:416-425.
Rubin et al. (1980) Partial nucleotide sequence of the 300-nucleotide interspersed repeated human DNA sequences. Nature 284:372-374.
Sarks et al. (1988) Evolution of geographic atrophy of the retinal pigment epithelium. Eye (Lond) 2 (Pt 5):552-577.
Seo et al. (2015) Intestinal macrophages arising from CCR2(+) monocytes control pathogen infection by activating innate lymphoid cells. Nat Commun 6:8010.
Shen et al. (1997) BC1 RNA, the transcript from a master gene for ID element amplification, is able to prime its own reverse transcription. Nucleic Acids Res 25:1641-1648.
Shi et al. (2007). Cell Divisions Are Required for L1 Retrotransposition. Mol Cell Biol 27:1264-1270.
Sinibaldi-Vallebona et al. (2011). Retrotransposon-Encoded Reverse Transcriptase in the Genesis, Progression and Cellular Plasticity of Human Cancer. Cancers 3:1141-1157.
Stetson et al. (2008) Trex1 prevents cell-intrinsic initiation of autoimmunity. Cell 134:587-598.
Tarallo et al. (2012) DICER1 loss and Alu RNA induce age-related macular degeneration via the NLRP3 inflammasome and MyD88. Cell 149:847-859.
Taylor et al. (2013) Affinity proteomics reveals human host factors implicated in discrete stages of LINE-1 retrotransposition. Cell 155:1034-48.
Temin & Mizutani (1970) RNA-dependent DNA polymerase in virions of Rous sarcoma virus. Nature 226:1211-1213.
Thomas et al. (2017) Modeling of TREX1-Dependent Autoimmune Disease using Human Stem Cells Highlights L1 Accumulation as a Source of Neuroinflammation. Cell Stem Cell 21:319-331.
Troegeler et al. (2014) An efficient siRNA-mediated gene silencing in primary human monocytes, dendritic cells and macrophages. Immunol Cell Biol 92:699-708.
Tyagi et al. (2017) Inhibition of human endogenous retrovirus-K by antiretroviral drugs. Retrovirology 14:21.
U.S. Patent Application Publication Nos. 2003/0124186; 2005/0223427; 2005/0255487; 2009/0099060; 2018/0044327; 2019/0184018.
U.S. Pat. Nos. 5,093,246; 5,633,133; 6,251,588; 9,421,204; 10,214,591; 10,294,220; 10,371,703.
Venter et al. (2001) The sequence of the human genome. Science 291:1304-1351.
Wagstaff et al. (2012) Rescuing Alu: recovery of new inserts shows LINE-1 preserves Alu activity through A-tail expansion. PLoS Genet 8:e1002842.
Wakimoto et al. (2014) Isolation of single-stranded DNA. Curr Protoc Mol Biol 107:2.15.11-19.
Wallace et al. (2008) LINE-1 ORF1 protein enhances Alu SINE retrotransposition. Gene 419:1-6.
Wang et al. (2010) MapSplice: accurate mapping of RNA-seq reads for splice junction discovery. Nucleic Acids Res 38:e178.
Wei et al. (2001) Human L1 retrotransposition: cis preference versus trans complementation. Mol Cell Biol 21:1429-1439.
Weiss (2016) Human endogenous retroviruses: friend or foe? APMIS 124:4-10.
Wong et al. (2014) Global prevalence of age-related macular degeneration and disease burden projection for 2020 and 2040: a systematic review and meta-analysis. Lancet Glob Health 2:e106-116.
Yan et al. (2013) Dicer expression exhibits a tissue-specific diurnal pattern that is lost during aging and in diabetes. PLoS One 8:e80029.
Yang et al. (2007) Trex1 exonuclease degrades ssDNA to prevent chronic checkpoint activation and autoimmune disease. Cell 131:873-886.
Yang et al. (2014) Reviving the dead: history and reactivation of an extinct 11. PLoS Genet 10:e1004395.

It will be understood that various details of the presently disclosed subject matter may be changed without departing from the scope of the presently disclosed subject matter. Furthermore, the foregoing description is for the purpose of illustration only, and not for the purpose of limitation.

1. A method for treating age-related macular degeneration (AGE), or preventing the occurrence or progression thereof, the method comprising administering to a subject in need thereof a composition comprising an effective amount of an inhibitor of reverse transcriptase (RTase) activity.

2. The method of claim 1, wherein the RTase activity is cytoplasmic RTase activity.

3. The method of claim 1, wherein the inhibitor reduces cytoplasmic accumulation of a reverse transcription product of an Alu nucleic acid, optionally wherein the reverse transcription product is a single-stranded Alu cDNA.

4. The method of claim 1, wherein the inhibitor of RTase activity is an inhibitor of an L1 ORF2 polypeptide RTase activity.

5. The method of claim 1, wherein the inhibitor of RTase activity is selected from the group consisting of an L1 ORF2 inhibitor, a nucleoside reverse transcriptase inhibitor (NRTI), an alkylated derivative of an NRTI, and a non-nucleoside reverse transcriptase inhibitor (NNRTI).

6. The method of claim 5, wherein the L1 ORF2 inhibitor is selected from the group consisting of an inhibitory nucleic acid that targets an L1 ORF2 transcription product and an antibody that is specific for an L1 ORF2 polypeptide.

7. The method of claim 6, wherein the L1 ORF2 polypeptide is a human L1 ORF2 polypeptide, optionally comprising an amino acid sequence as set forth in SEQ ID NO: 57.

8. The method of claim 5, wherein the NNRTI is selected from the group consisting of efavirenz (EFV) and delaviridine (DLV).

9. The method of claim 1, wherein the composition is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration; or intravenous administration.

10. The method of claim 1, wherein the composition comprises an effective amount of a cell-permeable, non-immunogenic cholesterol-conjugated siRNA that targets an L1 ORF2-encoding nucleic acid, optionally wherein the siRNA comprises, consists essentially of, or consists of any one of SEQ ID NOs: 47-49 and 51-54.

11. A method for protecting a retinal pigmented epithelium (RPE) cell, a retinal photoreceptor cell, or a choroidal cell, the method comprising administering to a subject in need thereof a composition comprising an effective amount of an inhibitor of reverse transcriptase (RTase) activity.

12. The method of claim 11, wherein the RTase activity is cytoplasmic RTase activity.

13. The method of claim 11, wherein the inhibitor reduces cytoplasmic accumulation of a reverse transcription product of an Alu nucleic acid, optionally wherein the reverse transcription product is a single-stranded Alu cDNA.

14. The method of claim 11, wherein the inhibitor of RTase activity is an inhibitor of an L1 ORF2 polypeptide RTase activity.

15. The method of claim 11, wherein the inhibitor of RTase activity is selected from the group consisting of an L1 ORF2 inhibitor, a nucleoside reverse transcriptase inhibitor (NRTI), an alkylated derivative of an NRTI, and a non-nucleoside reverse transcriptase inhibitor (NNRTI).

16. The method of claim 15, wherein the L1 ORF2 inhibitor is selected from the group consisting of an inhibitory nucleic acid that targets an L1 ORF2 transcription product and an antibody that is specific for an L1 ORF2 polypeptide.

17. The method of claim 16, wherein the L1 ORF2 polypeptide is a human L1 ORF2 polypeptide, optionally comprising an amino acid sequence as set forth in SEQ ID NO: 57.

18. The method of claim 15, wherein the NNRTI is selected from the group consisting of efavirenz (EFV) and delaviridine (DLV).

19. (canceled)

20. The method of claim 11, wherein the composition comprises an effective amount of a cell-permeable, non-immunogenic cholesterol-conjugated siRNA that targets an L1 ORF2-encoding nucleic acid, optionally wherein the siRNA comprises, consists essentially of, or consists of any one of SEQ ID NOs: 47-49 and 51-54.

21. A method for treating geographic atrophy (GA) of the eye, or preventing occurrence or progression thereof, the method comprising administering to a subject in need thereof a composition comprising an effective amount of an inhibitor of reverse transcriptase (RTase) activity.

22-40. (canceled)
